(19) United States
(12) Patent Application Publication
(10) Pub. No.: US 2026/0209242 A1
(43) Pub. Date: Jul. 23, 2026

(54) CAMPTOTHECIN DERIVATIVE, PHARMACEUTICAL COMPOSITION, AND PREPARATION METHOD AND USE THEREFOR

(71) Applicant: CHANGCHUN GENESCIENCE PHARMACEUTICAL CO., LTD., Changchun, Jilin (CN)

(72) Inventors: Fanglong YANG, Changchun, Jilin (CN); Yue HUANG, Changchun, Jilin (CN); Weiming HE, Changchun, Jilin (CN); Kaiqin DAI, Changchun, Jilin (CN); Siqin WANG, Changchun, Jilin (CN); Lei JIN, Changchun, Jilin (CN)

(21) Appl. No.: 19/136,561

(22) PCT Filed: Dec. 28, 2023
(86) PCT No.: PCT/CN2023/142848

§ 371 Date: Jun. 6, 2025

(30) Foreign Application Priority Data
Dec. 30, 2022 (CN) 202211739606.8
Apr. 7, 2023 (CN) 202310367781.7
Jun. 21, 2023 (CN) 202310743291.2
Jul. 28, 2023 (CN) 202310941713.7
Sep. 12, 2023 (CN) 202311173712.9
Dec. 25, 2023 (CN) 202311802330.8

(51) Int. Cl. C07D 491/22; A61K 31/4745; A61K 47/68; A61P 35/00

(52) U.S. Cl. CPC C07D 491/22; A61K 31/4745; A61K 47/68037; A61P 35/00

(57) ABSTRACT

A camptothecin derivative as shown in general formula I, a pharmaceutical composition, and a preparation method and use therefor are provided. The camptothecin derivative has good tumor inhibitory activity, can be used for treating or preventing tumors, and can be used for preparing drugs for treating or preventing tumor diseases.

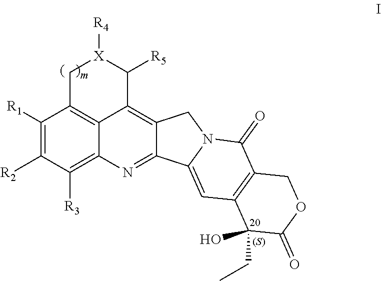

The present application claims priority to application No. 202211739606.8 filed on 30 Dec. 2022, application No. 202310367781.7 filed on 7 Apr. 2023, application No. 202310743291.2 filed on 21 Jun. 2023, and application No. 202310941713.7 filed on 28 Jul. 2023, application No. 202311173712.9 filed on 12 Sep. 2023, and application No. 202311802330.8 filed on 25 Dec. 2023, which are all titled “CAMPTOTHECIN DERIVATIVE, PHARMACEUTICAL COMPOSITION, PREPARATION METHOD THEREFOR, AND USE THEREOF”.

### TECHNICAL FIELD

The present invention belongs to the field of pharmaceuticals, and specifically relates to a camptothecin derivative, a pharmaceutical composition, a preparation method therefor, and use thereof.

### BACKGROUND

Camptothecin (CPT) is a natural product isolated from Camptotheca acuminata, a plant of nyssaceae. Camptothecin is a pentacyclic compound composed of quinoline ring AB, pyrrole ring C, pyridone ring D, and α-hydroxylactone ring E, in which the 20-C is S-configuration (see the structural formula below). Due to its excellent anti-cancer activity, it was put into clinical use in the early 1970s. Later, severe drug side effects such as diarrhea and hemorrhagic cystitis occurred clinically, and the clinical trial was terminated.

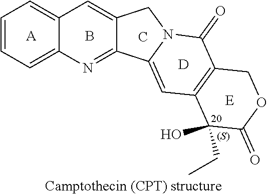

Research data shows that camptothecin can form a ternary complex with DNA topoisomerase I in cells, thereby inhibiting DNA despiralization, blocking DNA replication, and resulting in cell death (Cancer Res. 1989, 49, 6365). Camptothecin and a derivative thereof have very strong anti-tumor activity in an in vivo animal model such as lung cancer, breast cancer, colorectal cancer, and ovarian cancer (Nature Review Cancer. 2006, 6, 789). At present, a plurality of camptothecin drugs have been approved for marketing for tumor treatment (Med Res. Rev. 2015, 35, 753). Irinotecan is a medicament for treating colorectal cancer; topotecan is used for treating ovarian cancer; and belotecan is used for treating ovarian cancer and small cell lung cancer. The camptothecin derivative further comprises, e.g., exatecan, rubitecan, diflomotecan, lurtotecan, gimatecan, simimtecan, chimmitecan, or elomotecan. The camptothecin drugs or derivatives often have hematotoxicity caused by myelosuppression, such as leukopenia, thrombocytopenia, anemia, or neutropenia, as well as gastrointestinal side effects, such as nausea, emesis, or diarrhea. As found through clinical studies, measures for improving the safety and efficacy of camptothecin compounds comprise, e.g., increasing water solubility, improving pharmacokinetic properties thereof, enhancing activity, reducing dosage, or forming antibody-drug conjugates using conjugates thereof and antibodies. Therefore, research and development of camptothecin compounds and conjugates thereof with novel structures and capable of enhancing efficacy and improving safety issues still has very high clinical demand and application value.

### SUMMARY OF THE INVENTION

The present invention provides a compound represented by formula I, a racemate, a stereoisomer, a tautomer, an isotopically labeled compound, a solvate, a polymorph, a pharmaceutically acceptable salt, or a prodrug compound thereof:

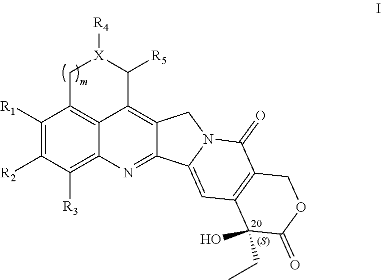

wherein R₁, R₂, and R₃ are identical or different, and are selected independently of each other from H, OH, CN, a halogen, a C₁₋₁₀ alkyl, a C₂₋₁₀ alkenyl, a C₂₋₁₀ alkynyl, a C₁₋₁₀ alkoxy, a C₁₋₁₀ haloalkyl, a C₁₋₁₀ haloalkoxy, a C₁₋₁₀ cyanoalkyl, a C₁₋₁₀ cyanoalkoxy, or a C₃₋₁₀ cycloalkyl;
R₄ is selected from H or

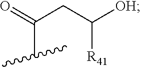

R₄₁ is selected from H, a C₁₋₆ alkyl, a C₁₋₆ haloalkyl, a C₁₋₆ alkyl-NH—, a (C₁₋₆ alkyl)₂N—, a C₁₋₆ alkyl-NH—C₁₋₆ alkyl, a (C₁₋₆ alkyl)₂N—C₁₋₆ alkyl, a C₁₋₆ alkoxyalkyl, a C₂₋₆ alkenyl, a C₂₋₆ alkynyl, a C₃₋₆ cycloalkyl, a 3-6-membered heterocyclyl, a C₆₋₁₄ aryl, and a 5-14-membered heteroaryl;

R₅ is selected from H,

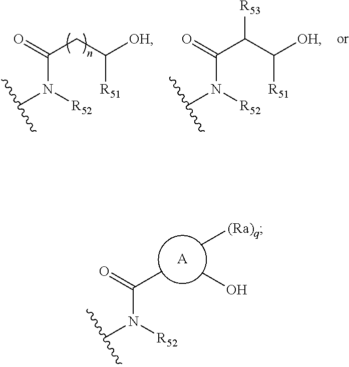

R₅₁ and R₅₂ are identical or different, and are selected independently of each other from H, a C₁₋₆ alkyl, a C₁₋₆ haloalkyl, a C₁₋₆ alkyl-NH—, a (C₁₋₆ alkyl)₂N—, a C₁₋₆ alkyl-NH—C₁₋₆ alkyl, a (C₁₋₆ alkyl)₂N—C₁₋₆ alkyl, a C₁₋₆ alkoxyalkyl, a C₂₋₆ alkenyl, a C₂₋₆ alkynyl, a C₃₋₆ cycloalkyl, a 3-6-membered heterocyclyl, a C₆₋₁₄ aryl, and a 5-14-membered heteroaryl; R₅₃ is selected from a C₁₋₆ alkyl, a C₁₋₆ haloalkyl, a C₁₋₆ alkyl-NH—, a (C₁₋₆ alkyl)₂N—, a C₁₋₆ alkyl-NH—C₁₋₆ alkyl, a (C₁₋₆ alkyl)₂N—C₁₋₆ alkyl, a C₁₋₆ alkoxyalkyl, a C₂₋₆ alkenyl, a C₂₋₆ alkynyl, a C₃₋₆ cycloalkyl, a 3-6-membered heterocyclyl, a C₆₋₁₄ aryl, and a 5-14-membered heteroaryl; ring A is selected from a C₃₋₉ cycloalkyl or a C3-8 heterocyclyl, Ra is selected from H, hydroxyl, CN, a halogen, a C₁₋₆ alkyl, or a C₁₋₆ haloalkyl; n is selected from 0, 1, or 2; and q is selected from 0, 1, or 2;
X is selected from CH or N; and
m is an integer selected from 0-6.

According to some embodiments, the R₁ is selected from H, OH, CN, a halogen, a C₁₋₆ alkyl, a C₂₋₆ alkenyl, a C₂₋₆ alkynyl, a C₃₋₆ cycloalkyl, or a C₁₋₆ haloalkoxy,
according to some embodiments, the R₁ is selected from H, OH, CN, a halogen, a C₁₋₆ alkyl, a C₂-6 alkenyl, a C₂₋₆ alkynyl, or a C₁₋₆ haloalkoxy;
according to some embodiments, the R₁ is selected from H, OH, Br, methyl, difluoromethoxy, 2,2,2-trifluoroethoxy, ethenyl, cyclopropyl, or ethynyl; and
according to some embodiments, the R₁ is selected from H, OH, Br, methyl, difluoromethoxy, 2,2,2-trifluoroethoxy, ethenyl, or ethynyl.

According to some embodiments, the R₂ is selected from H, a halogen, CN, or a C₁₋₆ alkyl; and according to some embodiments, the R₂ is selected from H or F.

According to some embodiments, the R₃ is selected from H or a C₁₋₆ alkyl; and according to some embodiments, the R₃ is H.
according to some embodiments, the R₄ is selected from H or

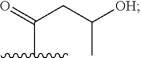 [SMILES: *C(=O)CC(C)O]

for example,

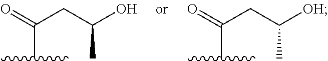

and
according to some embodiments, the R₄ is selected from H or

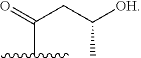 [SMILES: *C(=O)C[C@@H](C)O]

According to some embodiments, X—R₄ is

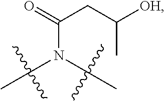 [SMILES: *N(*)C(=O)CC(C)O]

and is preferably

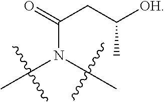 [SMILES: *N(*)C(=O)C[C@@H](C)O]

According to some embodiments, the X—R₄ is —CH₂—.

According to some embodiments, the R₅ is selected from H,

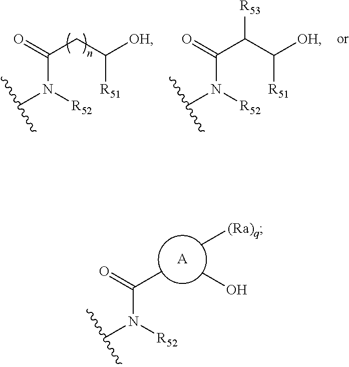

wherein the R₅₁ is selected from H, methyl, ethyl, isopropyl, or cyclopropyl; the R₅₂ is selected from H or methyl: the R₅₃ is selected from methyl; the ring A is selected from a C₃₋₆ cycloalkyl; the Ra is selected from H, hydroxyl, CN, a halogen, a C₁₋₆ alkyl, or a C₁₋₆ haloalkyl; the n is selected from 0 or 1; and the q is selected from 0 or 1; and
according to some embodiments, the ring A is selected from a cyclobutane ring.

According to some embodiments, the R₅ is selected from H,

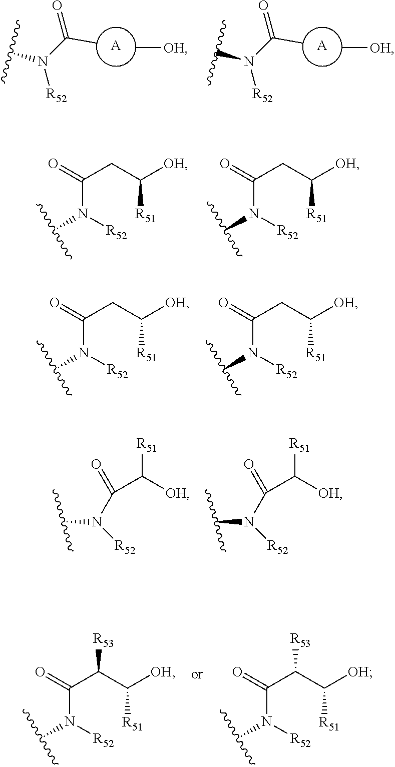

according to some embodiments, the R₅₁ is selected from H, methyl, ethyl, isopropyl, or cyclopropyl: the R₅₂ is selected from H or methyl; the R₅₃ is selected from methyl; and the ring A is selected from a cyclobutane ring; and
according to some embodiments, the R₅ is selected from H,

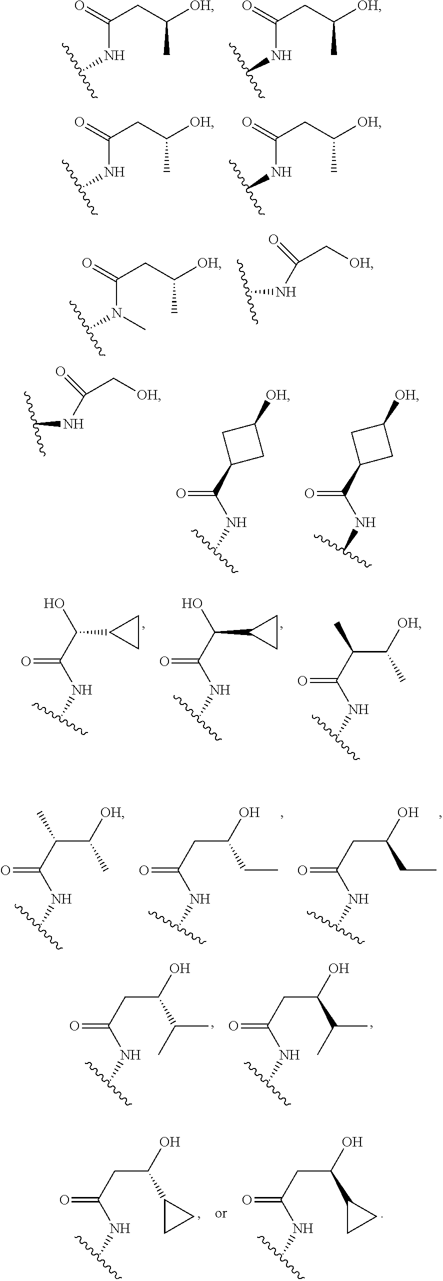

According to some embodiments, the R₅ is selected from H,

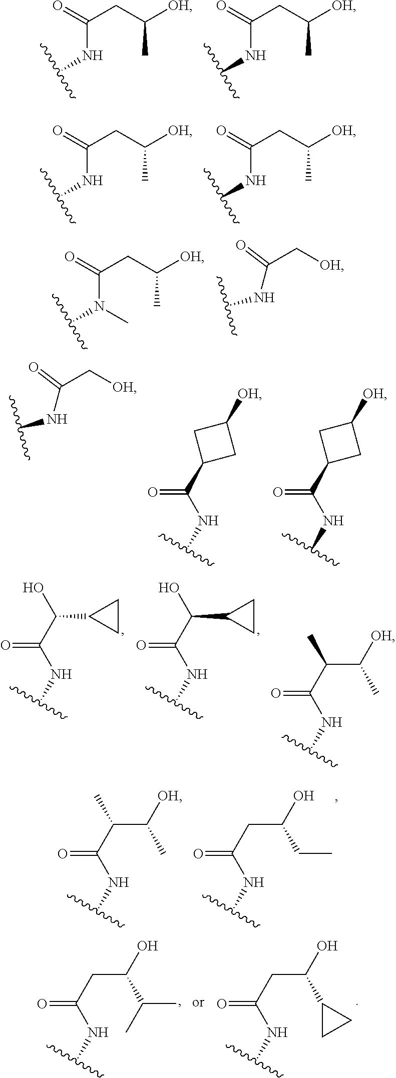

According to some embodiments, the X is selected from CH or N; and when X is CH, the R₄ is H; or when X is N, the R₅ is H; and
according to some embodiments, the m is selected from 0, 1, or 2.

According to some embodiments, the compound represented by formula I has a structure as shown below:

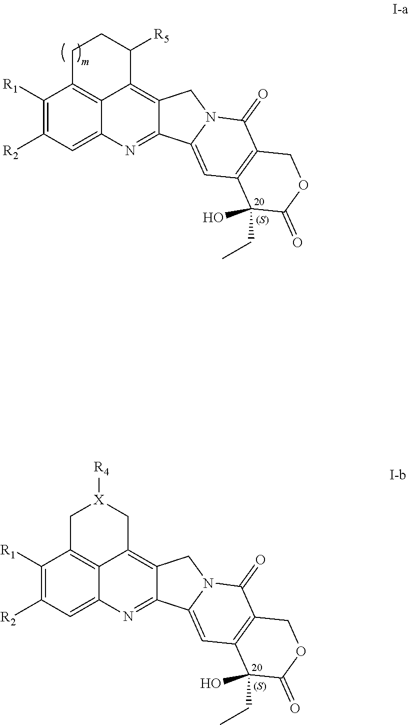

wherein the R₁, R₂, R₄, R₅, X, and m are as defined independently of each other herein.

According to some embodiments, the compound represented by formula I has a structure as shown below:

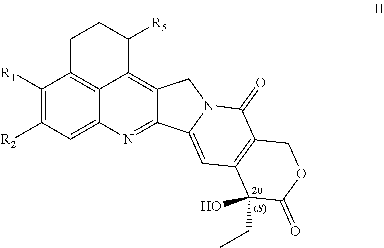

wherein the R₁, R₂, and R₅ are as defined independently of each other herein.

According to some embodiments, the compound represented by formula I has a structure as shown below:

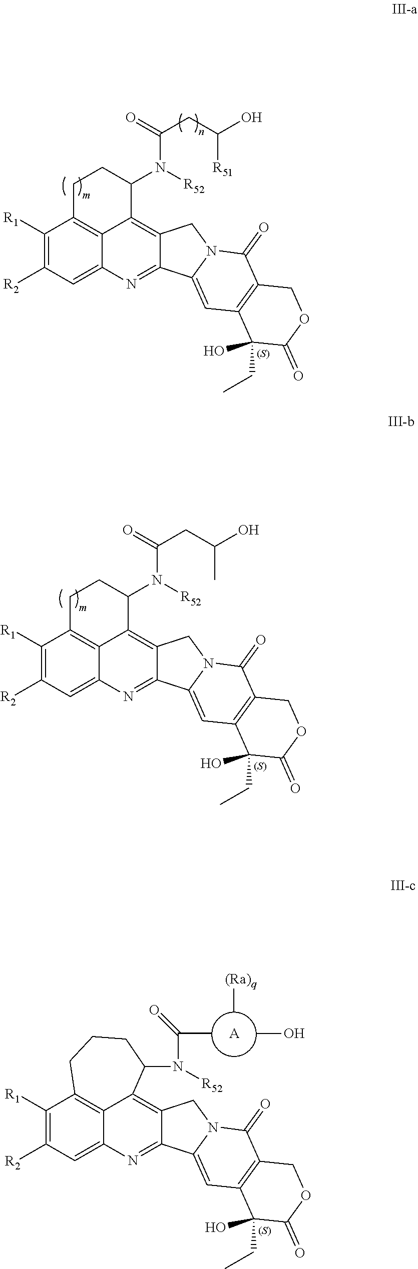

wherein the R₁, R₂, R₅₁, R₅₂, ring A, Ra, m, n, and q are as defined independently of each other herein.

According to some embodiments of the present invention, the compound of formula I has a structure as shown below:

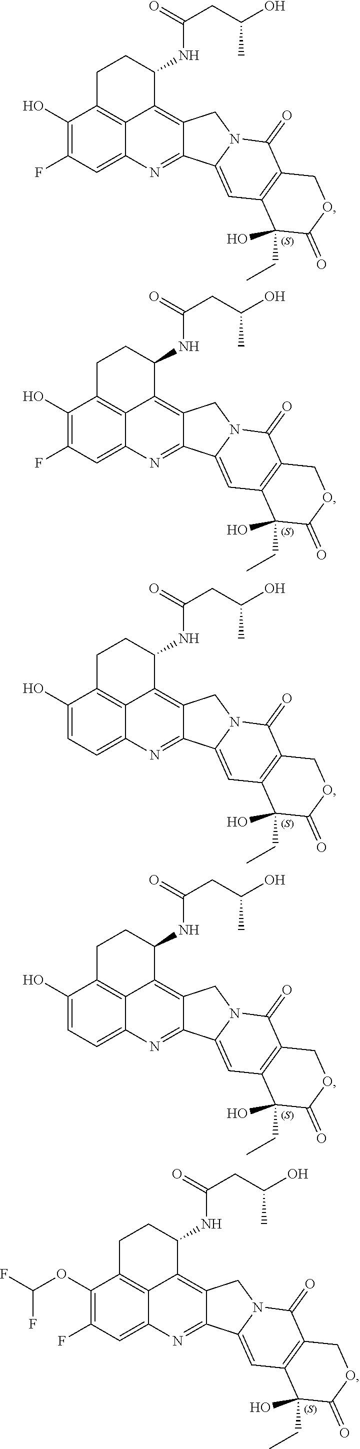

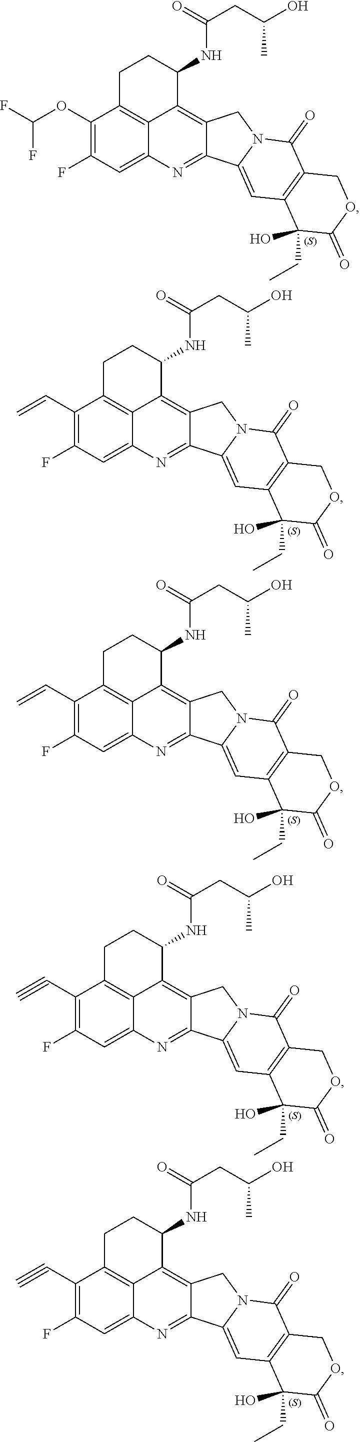

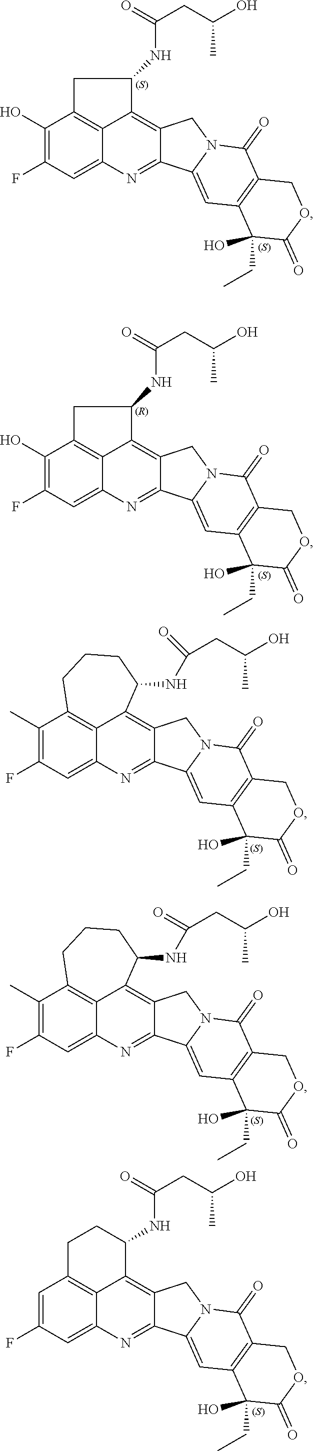

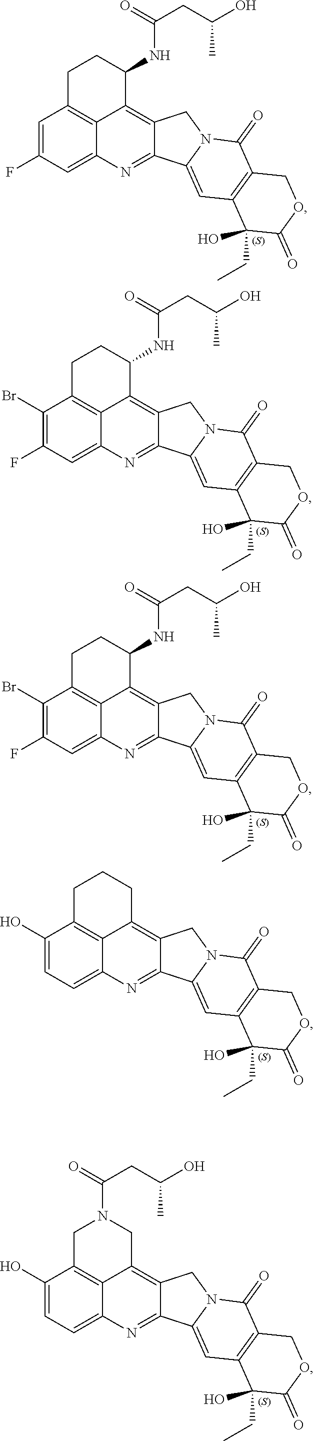

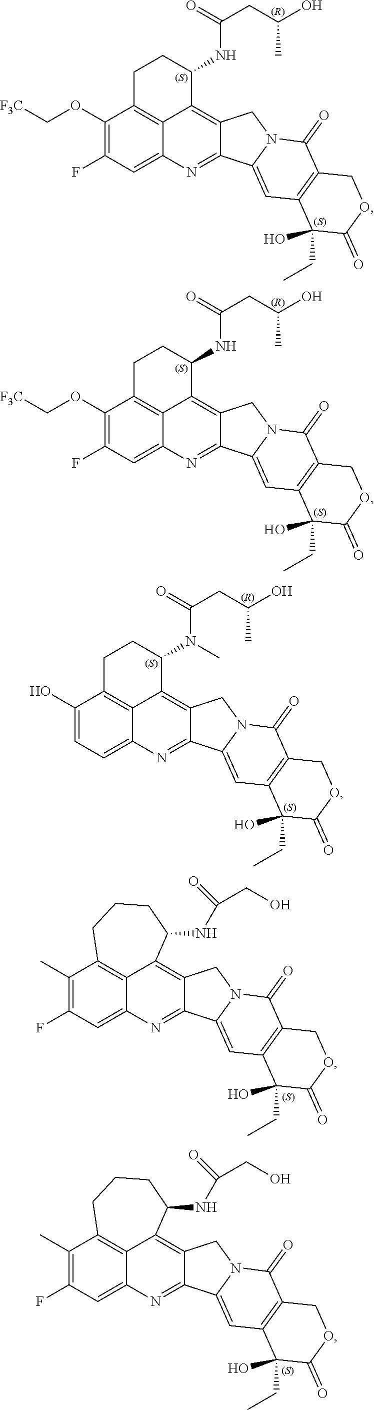

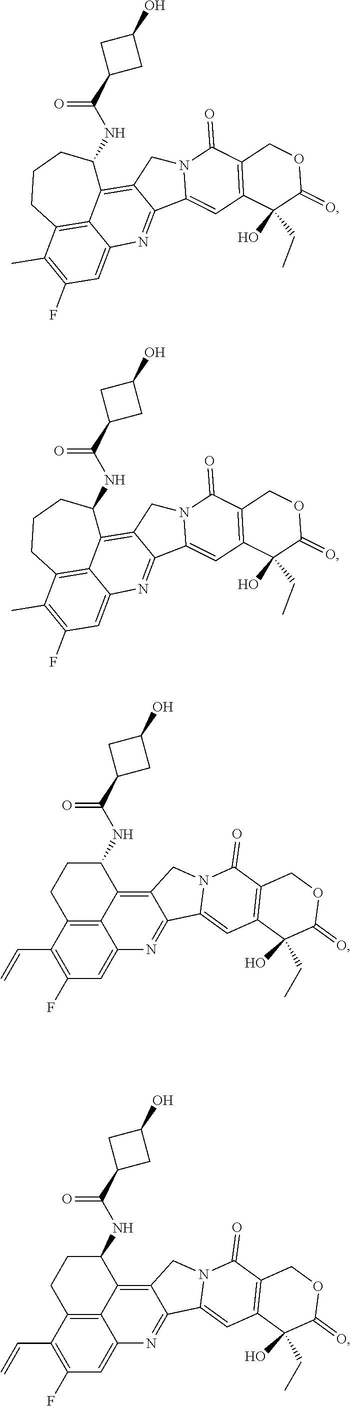 [SMILES: CC[C@@]1(O)C(=O)OCc2c1cc1n(c2=O)Cc2c-1nc1cc(F)c(C)c3c1c2[C@@H](NC(=O)[C@H]1C[C@@H](O)C1)CCC3 ; CC[C@@]1(O)C(=O)OCc2c1cc1n(c2=O)Cc2c-1nc1cc(F)c(C)c3c1c2[C@H](NC(=O)[C@H]1C[C@@H](O)C1)CCC3 ; C=Cc1c(F)cc2nc3c(c4c2c1CC[C@@H]4NC(=O)[C@H]1C[C@@H](O)C1)Cn1c-3cc2c(c1=O)COC(=O)[C@]2(O)CC ; C=Cc1c(F)cc2nc3c(c4c2c1CC[C@H]4NC(=O)[C@H]1C[C@@H](O)C1)Cn1c-3cc2c(c1=O)COC(=O)[C@]2(O)CC]

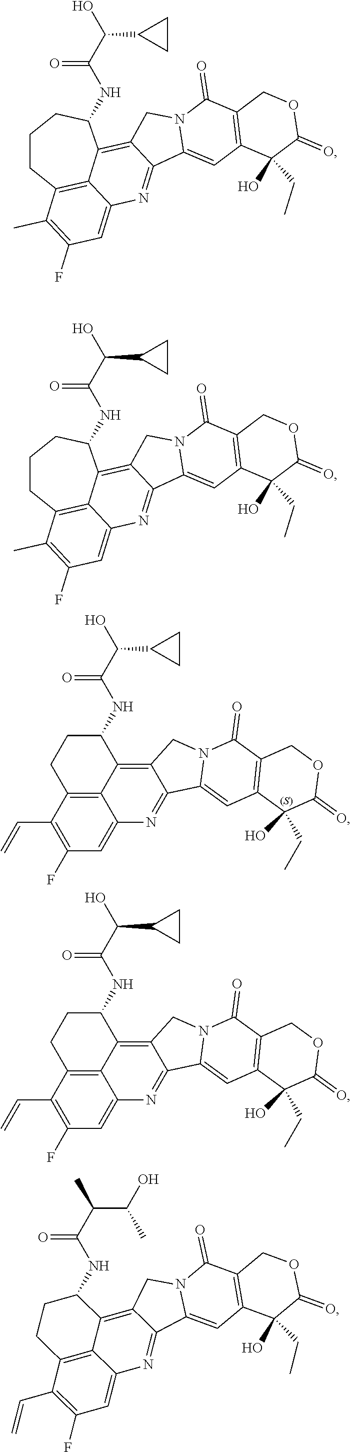

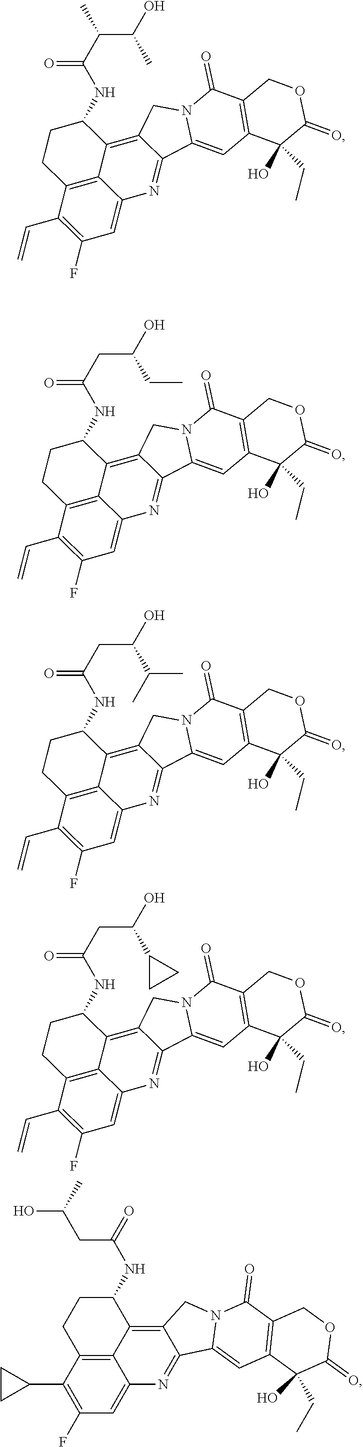

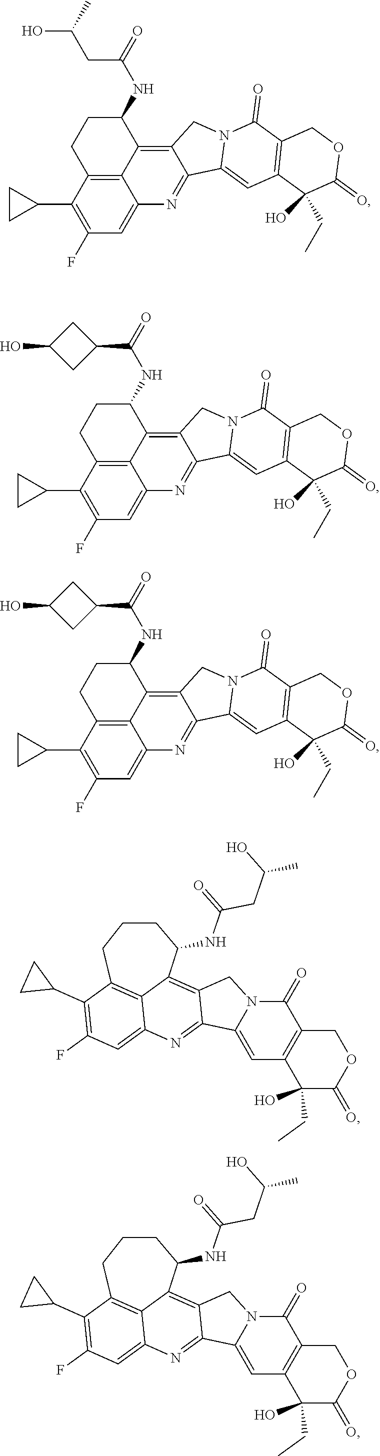

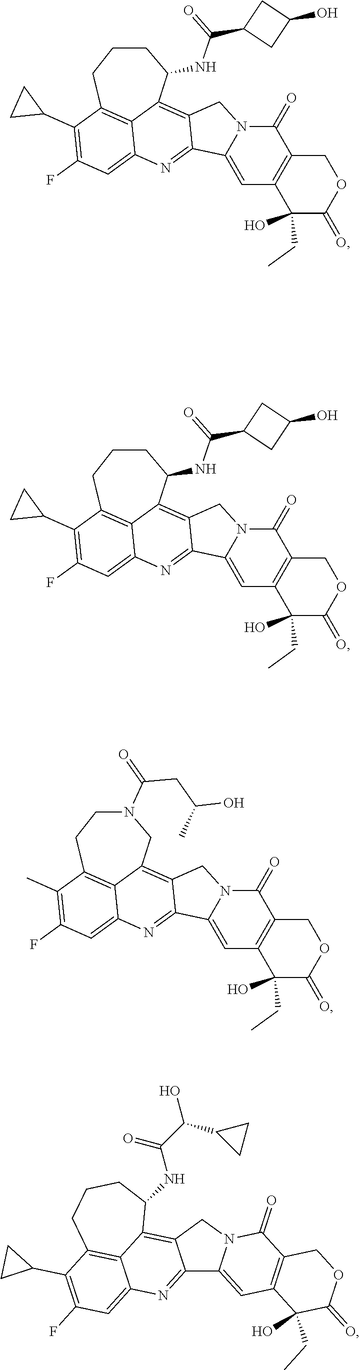 [SMILES: CC[C@@]1(O)C(=O)OCc2c1cc1n(c2=O)Cc2c-1nc1cc(F)c(C3CC3)c3c1c2[C@@H](NC(=O)[C@H]1C[C@@H](O)C1)CCC3 ; CC[C@@]1(O)C(=O)OCc2c1cc1n(c2=O)Cc2c-1nc1cc(F)c(C3CC3)c3c1c2[C@H](NC(=O)[C@H]1C[C@@H](O)C1)CCC3 ; CC[C@@]1(O)C(=O)OCc2c1cc1n(c2=O)Cc2c-1nc1cc(F)c(C)c3c1c2CN(C(=O)C[C@@H](C)O)CC3 ; CC[C@@]1(O)C(=O)OCc2c1cc1n(c2=O)Cc2c-1nc1cc(F)c(C3CC3)c3c1c2[C@@H](NC(=O)[C@H](O)C1CC1)CCC3]

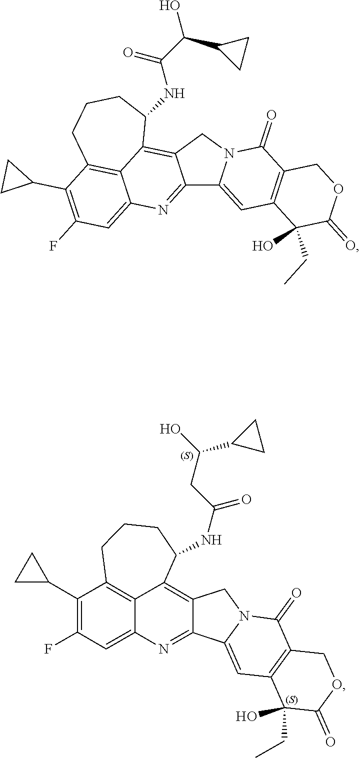

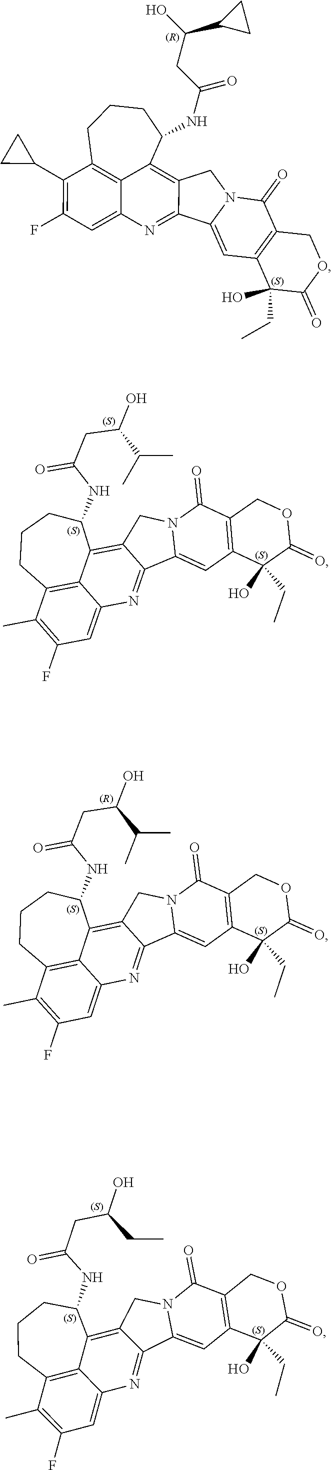

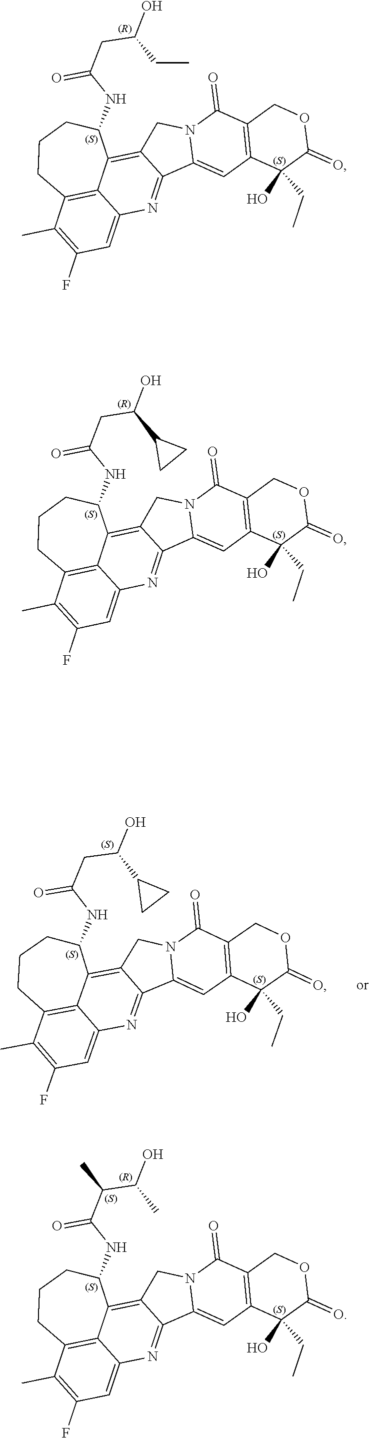

According to some embodiments, the present invention further provides a structural fragment D of the compound represented by formula I herein after dehydrogenation; and
according to some embodiments, the D has a structure as shown below:

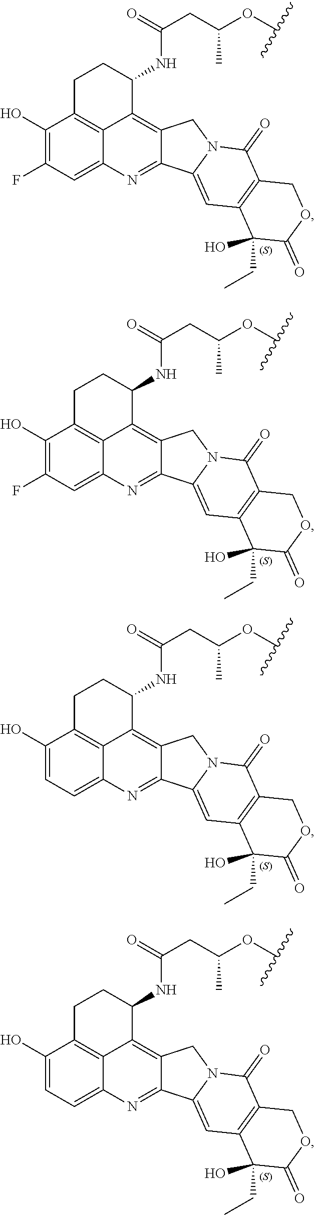

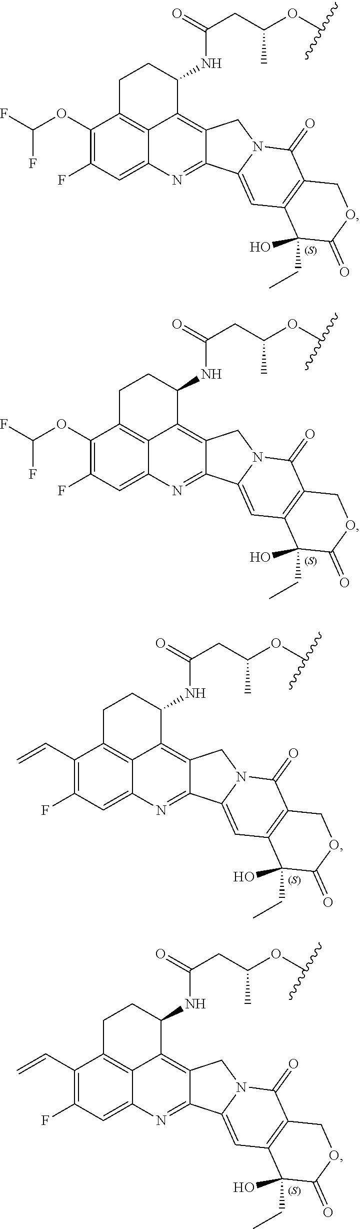

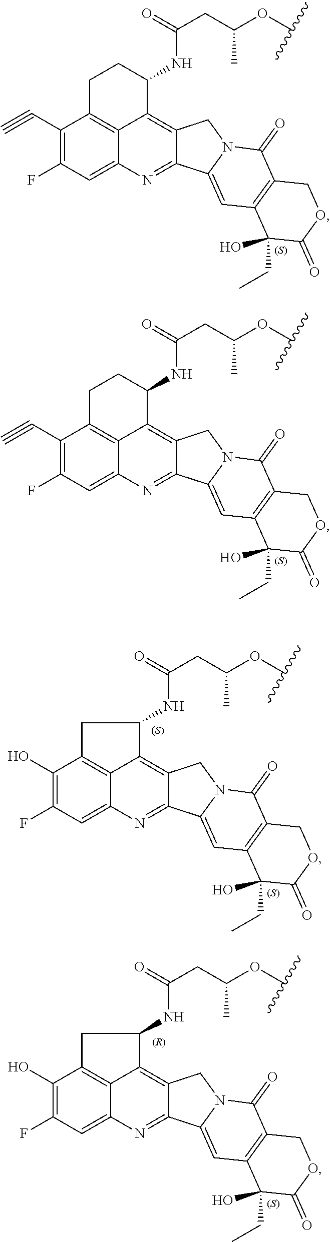

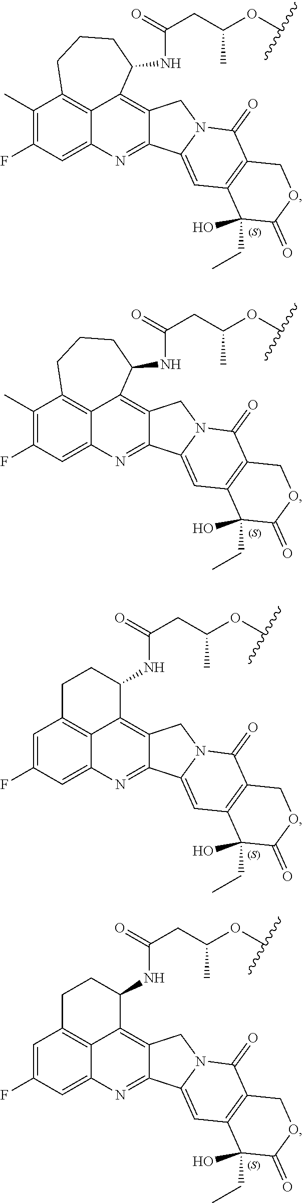

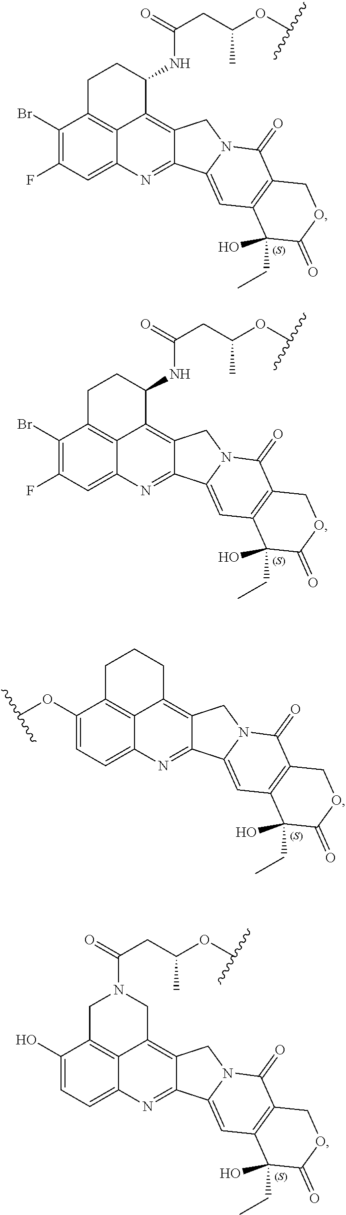

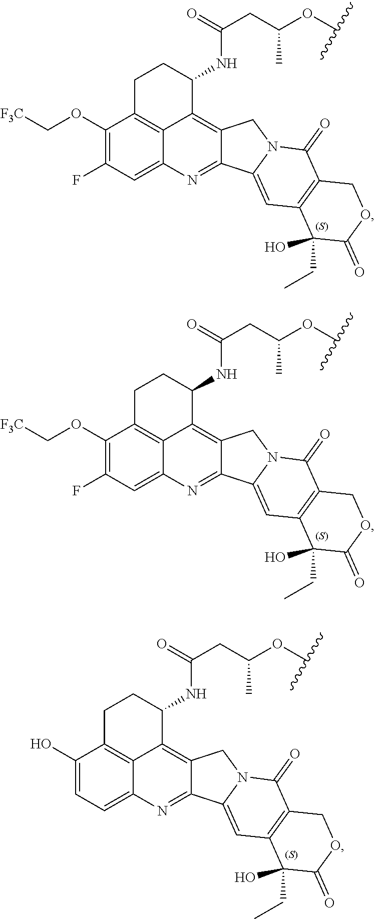

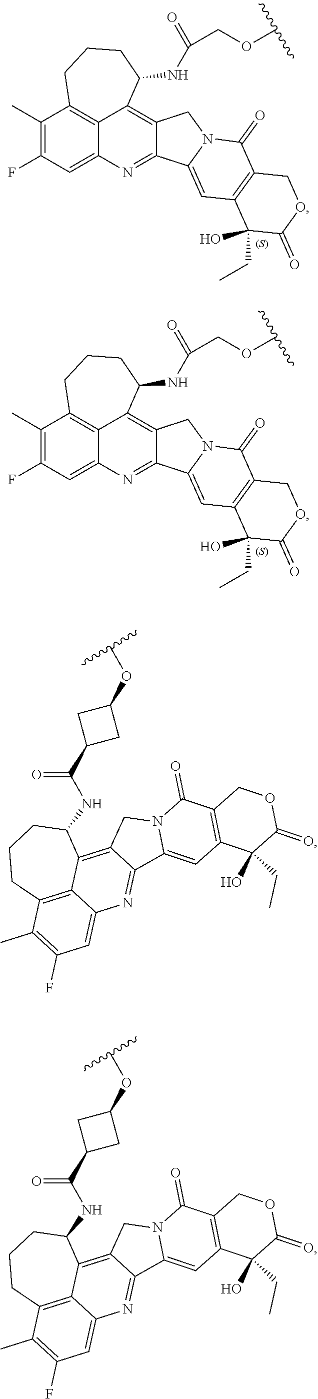

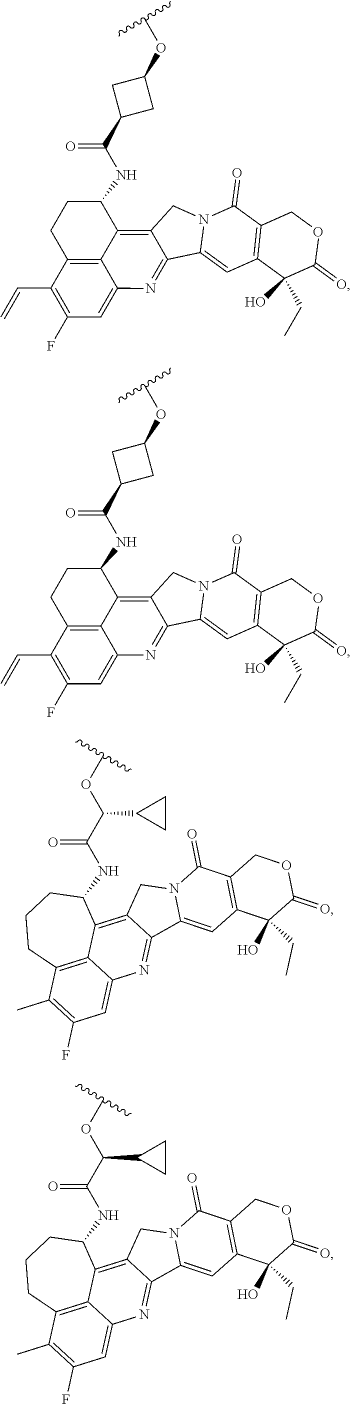 [SMILES: *O[C@H]1C[C@@H](C(=O)N[C@H]2CCc3c(C=C)c(F)cc4nc5c(c2c34)Cn2c-5cc3c(c2=O)COC(=O)[C@]3(O)CC)C1 ; *O[C@H]1C[C@@H](C(=O)N[C@@H]2CCc3c(C=C)c(F)cc4nc5c(c2c34)Cn2c-5cc3c(c2=O)COC(=O)[C@]3(O)CC)C1 ; *O[C@@H](C(=O)N[C@H]1CCCc2c(C)c(F)cc3nc4c(c1c23)Cn1c-4cc2c(c1=O)COC(=O)[C@]2(O)CC)C1CC1 ; *O[C@H](C(=O)N[C@H]1CCCc2c(C)c(F)cc3nc4c(c1c23)Cn1c-4cc2c(c1=O)COC(=O)[C@]2(O)CC)C1CC1]

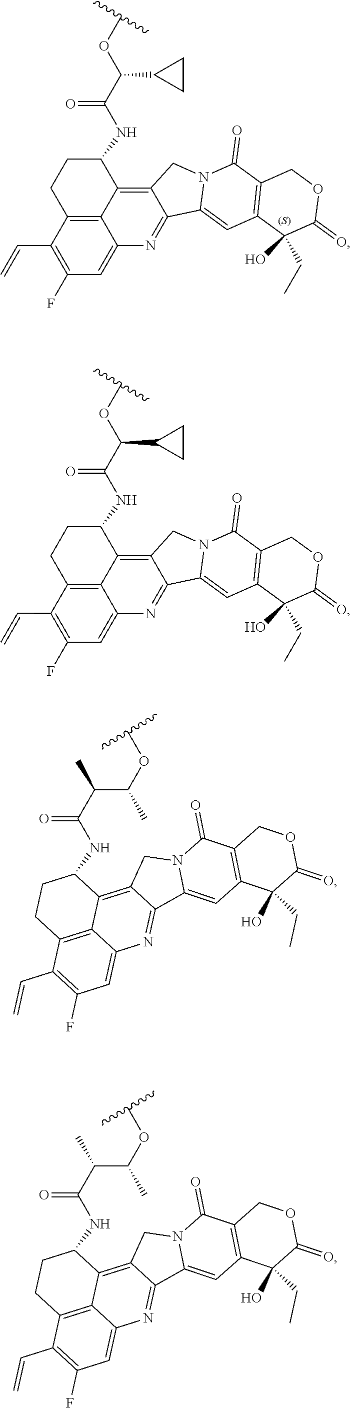

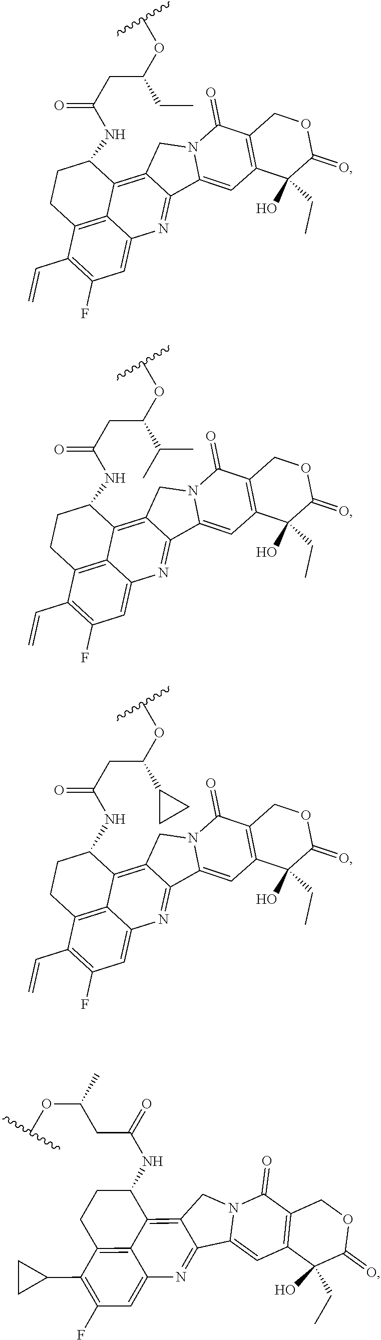 [SMILES: *O[C@H](CC)CC(=O)N[C@H]1CCc2c(C=C)c(F)cc3nc4c(c1c23)Cn1c-4cc2c(c1=O)COC(=O)[C@]2(O)CC ; *O[C@@H](CC(=O)N[C@H]1CCc2c(C=C)c(F)cc3nc4c(c1c23)Cn1c-4cc2c(c1=O)COC(=O)[C@]2(O)CC)C(C)C ; *O[C@@H](CC(=O)N[C@H]1CCc2c(C=C)c(F)cc3nc4c(c1c23)Cn1c-4cc2c(c1=O)COC(=O)[C@]2(O)CC)C1CC1 ; *O[C@H](C)CC(=O)N[C@H]1CCc2c(C3CC3)c(F)cc3nc4c(c1c23)Cn1c-4cc2c(c1=O)COC(=O)[C@]2(O)CC]

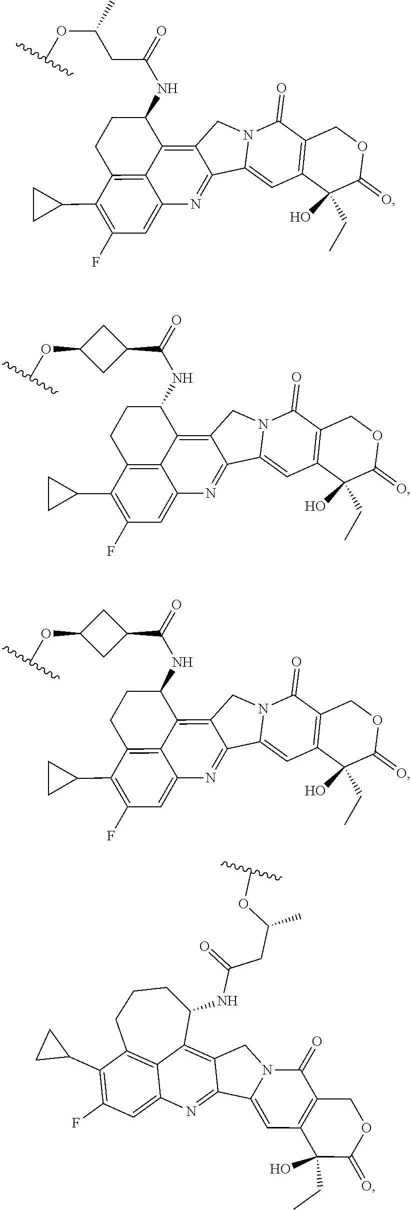 [SMILES: *O[C@H](C)CC(=O)N[C@@H]1CCc2c(C3CC3)c(F)cc3nc4c(c1c23)Cn1c-4cc2c(c1=O)COC(=O)[C@]2(O)CC ; *O[C@H]1C[C@@H](C(=O)N[C@H]2CCc3c(C4CC4)c(F)cc4nc5c(c2c34)Cn2c-5cc3c(c2=O)COC(=O)[C@]3(O)CC)C1 ; *O[C@H]1C[C@@H](C(=O)N[C@@H]2CCc3c(C4CC4)c(F)cc4nc5c(c2c34)Cn2c-5cc3c(c2=O)COC(=O)[C@]3(O)CC)C1 ; *O[C@H](C)CC(=O)N[C@H]1CCCc2c(C3CC3)c(F)cc3nc4c(c1c23)Cn1c-4cc2c(c1=O)COC(=O)[C@]2(O)CC]

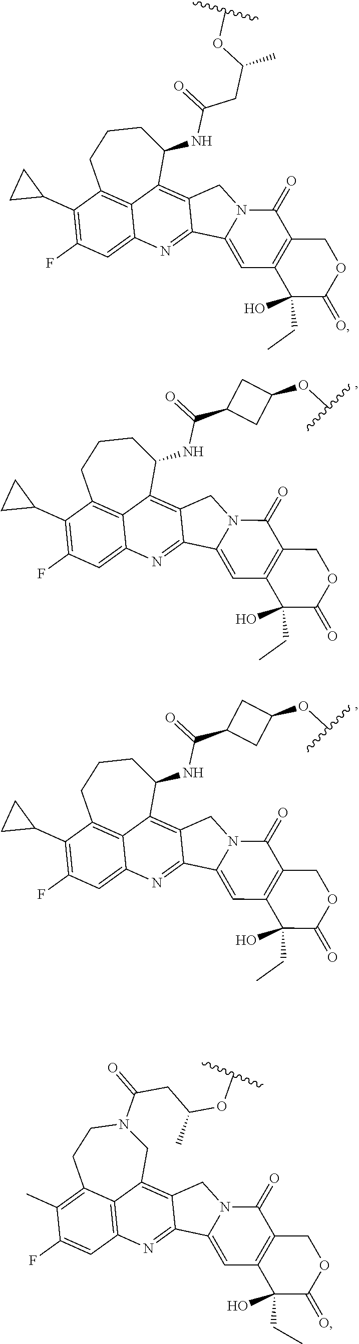

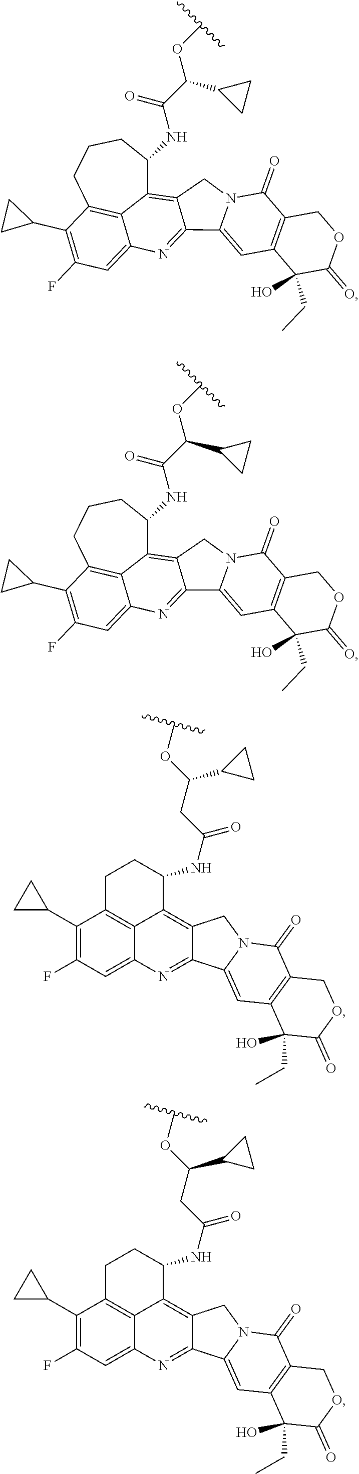 [SMILES: *O[C@@H](C(=O)N[C@H]1CCCc2c(C3CC3)c(F)cc3nc4c(c1c23)Cn1c-4cc2c(c1=O)COC(=O)[C@]2(O)CC)C1CC1 ; *O[C@H](C(=O)N[C@H]1CCCc2c(C3CC3)c(F)cc3nc4c(c1c23)Cn1c-4cc2c(c1=O)COC(=O)[C@]2(O)CC)C1CC1 ; *O[C@@H](CC(=O)N[C@H]1CCc2c(C3CC3)c(F)cc3nc4c(c1c23)Cn1c-4cc2c(c1=O)COC(=O)[C@]2(O)CC)C1CC1 ; *O[C@H](CC(=O)N[C@H]1CCc2c(C3CC3)c(F)cc3nc4c(c1c23)Cn1c-4cc2c(c1=O)COC(=O)[C@]2(O)CC)C1CC1]

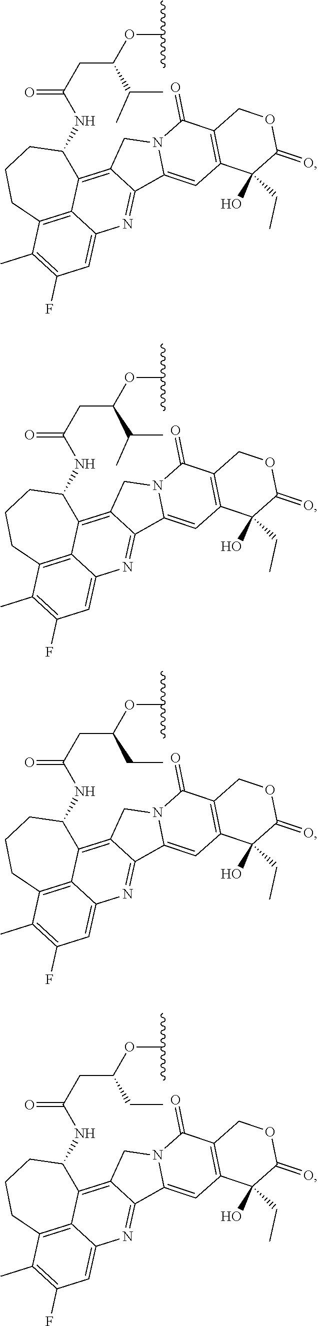 [SMILES: *O[C@@H](CC(=O)N[C@H]1CCCc2c(C)c(F)cc3nc4c(c1c23)Cn1c-4cc2c(c1=O)COC(=O)[C@]2(O)CC)C(C)C ; *O[C@H](CC(=O)N[C@H]1CCCc2c(C)c(F)cc3nc4c(c1c23)Cn1c-4cc2c(c1=O)COC(=O)[C@]2(O)CC)C(C)C ; *O[C@@H](CC)CC(=O)N[C@H]1CCCc2c(C)c(F)cc3nc4c(c1c23)Cn1c-4cc2c(c1=O)COC(=O)[C@]2(O)CC ; *O[C@H](CC)CC(=O)N[C@H]1CCCc2c(C)c(F)cc3nc4c(c1c23)Cn1c-4cc2c(c1=O)COC(=O)[C@]2(O)CC]

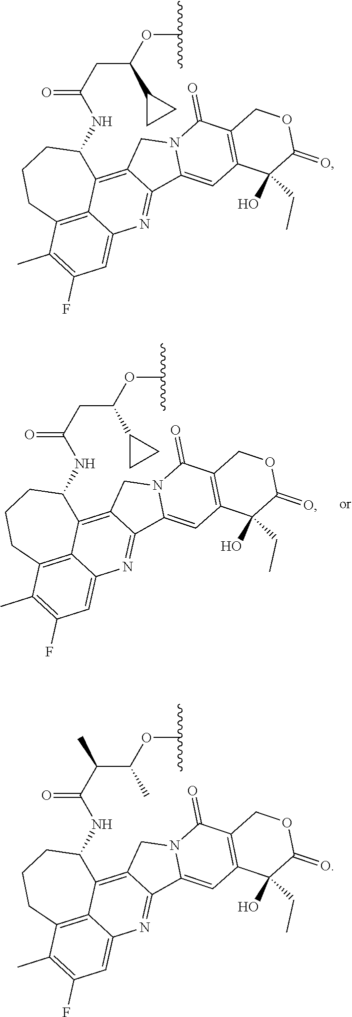 [SMILES: *O[C@H](CC(=O)N[C@H]1CCCc2c(C)c(F)cc3nc4c(c1c23)Cn1c-4cc2c(c1=O)COC(=O)[C@]2(O)CC)C1CC1 ; *O[C@@H](CC(=O)N[C@H]1CCCc2c(C)c(F)cc3nc4c(c1c23)Cn1c-4cc2c(c1=O)COC(=O)[C@]2(O)CC)C1CC1 ; "or" ; *O[C@H](C)[C@H](C)C(=O)N[C@H]1CCCc2c(C)c(F)cc3nc4c(c1c23)Cn1c-4cc2c(c1=O)COC(=O)[C@]2(O)CC]

The present invention further provides a compound represented by formula V, a racemate, a stereoisomer, a tautomer, an isotopically labeled compound, a solvate, a polymorph, a pharmaceutically acceptable salt, or a prodrug compound thereof:

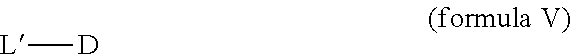

wherein L′ is a linker containing a linker site M capable of reacting with an antibody or an antigen-binding fragment thereof, and after L′-D reacts with the antibody or the antigen-binding fragment thereof, the L′ thereof forms a linker L; and
preferably, the L′ comprises a peptide residue L₁ and a fragment L₂ in which the peptide residue is connected to D.

The D is as defined herein.

The present invention further provides an antibody-drug conjugate represented by formula VI,

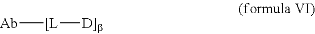

wherein Ab is an antibody or an antigen-binding fragment thereof, D is as defined in the text of the present application, L is a linker linking the Ab to the D, and 6 is an integer or a decimal selected from 1-10.

The present invention further provides a method for preparing the compound of formula I, comprising solution I or solution II below:
solution I: comprising steps of:
(1) removing a protecting group PG₄ from compound I-41 to give compound I-42; and
(2) allowing the compound I-42 to react with compound I-43 to give the compound represented by formula I;

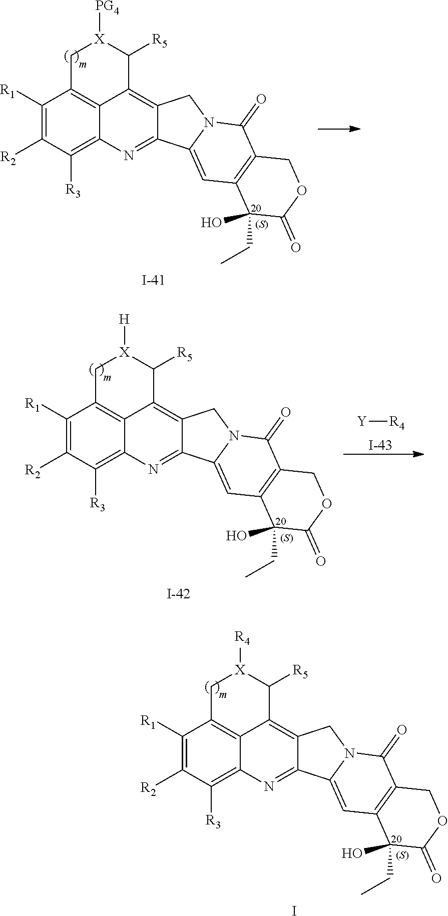

wherein the R₁, R₂, R₃, R₄, R₅, X, and m are as defined herein; Y is selected from a leaving group, for example, OH, Cl, Br, or I; and the PG₄ is selected from an amino protecting group, for example, Fmoc, Boc, Bn, or Cbz; or
solution II: comprising steps of:
(1) removing a protecting group PG₅ from compound I-51 to give compound I-52; and
(2) allowing the compound I-52 to react with compound I-53 to give the compound represented by formula I;

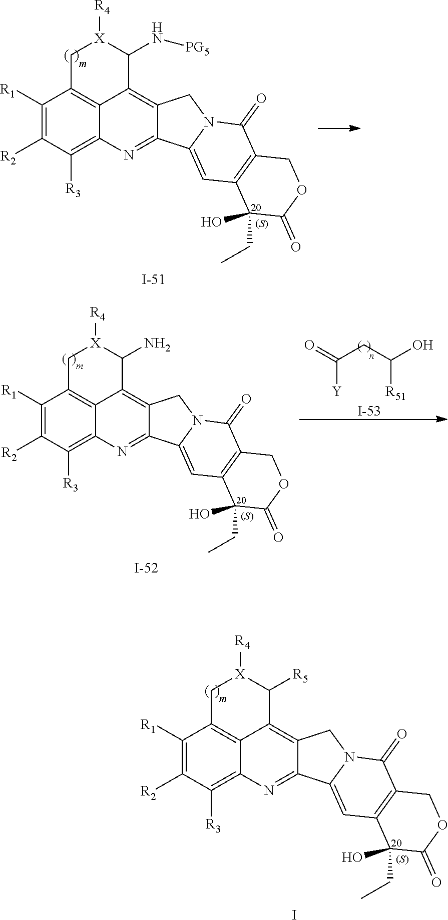

wherein the R₁, R₂, R₃, R₄, R₅, R₅₁, X, m, and n are as defined herein; Y is selected from a leaving group, for example, OH, Cl, Br, or I; and the PG is selected from an amino protecting group, for example, Fmoc, Boc, Bn, or Cbz.

The present invention further provides a pharmaceutical composition, comprising a therapeutically effective amount of at least one of the compound represented by formula I or formula V, a racemate, a stereoisomer, a tautomer, an isotopically labeled compound, a solvate, a polymorph, a pharmaceutically acceptable salt, or a prodrug compound thereof.

The present invention further provides a pharmaceutical composition, comprising a therapeutically effective amount of the antibody-drug conjugate represented by formula VI.

According to an embodiment of the present invention, the pharmaceutical composition further comprises one or more pharmaceutically acceptable excipients.

According to an embodiment of the present invention, the pharmaceutical composition may further comprise one or more additional therapeutic agents.

The present invention further provides a method for treating a tumor disease, comprising administering to a patient a prophylactically or therapeutically effective amount of at least one of a compound represented by formula I or formula V, or an antibody-drug conjugate represented by formula VI, a racemate, a stereoisomer, a tautomer, an isotopically labeled compound, a solvate, a polymorph, a pharmaceutically acceptable salt, or a prodrug compound thereof.

The present invention further provides a method for treating a tumor disease, comprising administering to a patient a prophylactically or therapeutically effective amount of the above pharmaceutical composition.

The tumor disease is selected from breast cancer, gastric cancer, lung cancer, colorectal cancer, large intestine cancer, ovarian cancer, liver cancer, kidney cancer, esophageal cancer, cervical cancer, bladder cancer, pancreatic cancer, prostate cancer, nasopharyngeal carcinoma, melanoma, or leukemia.

In some embodiments, the patient comprises a mammal, and is preferably a human.

The present invention further provides at least one of a compound represented by formula I or formula V, or an antibody-drug conjugate represented by formula VI, a racemate, a stereoisomer, a tautomer, an isotopically labeled compound, a solvate, a polymorph, a pharmaceutically acceptable salt, or a prodrug compound thereof, or a pharmaceutical composition thereof for treating a tumor disease.

The present invention further provides use of at least one of the compound represented by formula I or formula V, or the antibody-drug conjugate represented by formula VI, a racemate, a stereoisomer, a tautomer, an isotopically labeled compound, a solvate, a polymorph, a pharmaceutically acceptable salt, or a prodrug compound thereof, or the above pharmaceutical composition for the manufacture of a topoisomerase I inhibitor and/or for the manufacture of a medicament for preventing or treating a disease or condition associated with topoisomerase I.

In some embodiments, the disease or condition is a tumor, comprising breast cancer, gastric cancer, lung cancer, colorectal cancer, large intestine cancer, ovarian cancer, liver cancer, kidney cancer, esophageal cancer, cervical cancer, bladder cancer, pancreatic cancer, prostate cancer, nasopharyngeal carcinoma, melanoma, or leukemia.

### Beneficial Effects

The compound provided in the present invention has good tumor inhibiting effects, can be used for treating or preventing a cancer (for example, breast cancer or gastric cancer), and can be used for preparing a medicament for treating or preventing such a condition or disease.

### Definitions and Description of Terms

Unless otherwise indicated, the definitions of radicals and terms recorded in the specification and claims of the present application, comprising their definitions as examples, exemplary definitions, preferred definitions, definitions recorded in tables, definitions of specific compounds in examples, etc., may be arbitrarily combined and associated with each other. Such combined and associated radical definitions and compound structures should be understood to be within the scope of the specification and/or claims of the present application.

Unless otherwise indicated, the numerical ranges recited in the present specification and claims are equivalent to at least reciting each specific integer value therein. For example, the numerical range “1-12” is equivalent to reciting each integer value in the numerical range “1-12,” namely 1, 2, 3, 4, 5, 6, 7, 8, 9, 10, 11, and 12. In addition, when a numerical range is defined as a “number,” it should be understood that both endpoints of the range, each integer within the range, and each decimal within the range are recited.

The term “halogen” represents fluorine, chlorine, bromine, and iodine.

The “C₁₋₁₀ alkyl” represents a linear and branched alkyl having 1, 2, 3, 4, 5, 6, 7, 8, 9, or 10 carbon atoms, the “C₁₋₈ alkyl” represents a linear and branched alkyl having 1, 2, 3, 4, 5, 6, 7, or 8 carbon atoms, and the “C₁₋₆ alkyl” represents a linear and branched alkyl having 1, 2, 3, 4, 5, or 6 carbon atoms. The alkyl is, e.g., methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl, etc., or isomers thereof.

The “C₂₋₁₀ alkenyl” should be construed to represent preferably a linear or branched monovalent hydrocarbon radical comprising one or more double bonds and having 2, 3, 4, 5, 6, 7, 8, 9, or 10 carbon atoms, and is more preferably “C₂₋₈ alkenyl”. The “C₂₋₁₀ alkenyl” should be construed to represent preferably a linear or branched monovalent hydrocarbon radical comprising one or more double bonds and having 2, 3, 4, 5, 6, 7, or 8 carbon atoms, for example, having 2, 3, 4, 5, or 6 carbon atoms (namely, C₂₋₆ alkenyl) or having 2 or 3 carbon atoms (namely, C₂₋₃ alkenyl). It should be understood that in the case where the alkenyl comprises more than one double bond, the double bonds may be separated from each other or conjugated. The alkenyl is, e.g., ethenyl, allyl, (E)-2-methylethenyl, (Z)-2-methylethenyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propyethenyl, or 1-isopropylethenyl.

The “C₂₋₁₀ alkynyl” should be construed to represent preferably a linear or branched monovalent hydrocarbon radical comprising one or more triple bonds and having 2, 3, 4, 5, 6, 7, 8, 9, or 10 carbon atoms, for example, having 2, 3, 4, 5, 6, 7, or 8 carbon atoms (namely, “C₂₋₄ alkynyl”), having 2, 3, 4, 5, or 6 carbon atoms (namely, “C₂₋₆ alkynyl”), or having 2 or 3 carbon atoms (namely, “C₂₋₃ alkynyl”). The alkynyl is, e.g., ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl, or 3,3-dimethylbut-1-ynyl. In particular, the alkynyl is ethynyl, prop-1-ynyl, or prop-2-ynyl.

The term “C₃₋₁₀ cycloalkyl” should be construed to represent saturated monovalent monocyclic, bicyclic (e.g., bridged cyclic or spirocyclic) hydrocarbon ring or tricyclic alkane having 3, 4, 5, 6, 7, 8, 9, or 10 carbon atoms. The C₃₋₁₀ cycloalkyl may be a monocyclic hydrocarbon radical such as cyclopropyl, cyclobutyl, cyclopentyl, cyclohexyl, cycloheptyl, cyclooctyl, cyclononyl, or cyclodecyl, or may be a bicyclic hydrocarbon radical such as bornyl, indolyl, hexahydroindolyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl, 2,7-diazaspiro[3,5]nonyl, or 2,6-diazaspiro[3,4]octyl, or may be a tricyclic hydrocarbon radical such as adamantyl.

Unless otherwise defined, the term “3-6-membered heterocyclyl” refers to a saturated or unsaturated non-aromatic ring or ring system, for example, is a 4-, 5-, or 6-membered monocyclic ring, and contains at least one, for example, 1, 2, 3, 4, 5, or more heteroatoms selected from O, S, and N, wherein N and S may further be optionally oxidized to various oxidation states to form nitrogen oxides, —S(O)—, or —S(O)₂— states. The heterocyclyl may comprise a fused or bridged ring and a spirocyclic ring. In particular, the heterocyclyl may include, but is not limited to: a 4-membered ring, such as azetidinyl or oxetanyl; a 5-membered ring, such as tetrahydrofuryl, dioxolyl, pyrrolidyl, imidazolidinyl, pyrazolidyl, or pyrrolinyl; or a 6-membered ring, such as tetrahydropyranyl, piperidyl, morpholinyl, dithianyl, thiomorpholinyl, piperazinyl, or trithianyl.

The term “C₆₋₁₄ aryl” should be construed to represent preferably a monovalent aromatic or partially aromatic monocyclic, bicyclic, or tricyclic hydrocarbon ring having 6, 7, 8, 9, 10, 11, 12, 13, or 14 carbon atoms (“C₆₋₁₄ aryl”), particularly a ring having 6 carbon atoms (“C₆ aryl”), such as phenyl; or biphenyl, or a ring having 9 carbon atoms (“C₉ aryl”), such as indanyl or indenyl, or a ring having 10 carbon atoms (“C₁₀ aryl”), such as tetralyl, dihydronaphthyl, or naphthyl, or a ring having 13 carbon atoms (“C₁₃ aryl”), such as fluorenyl, or a ring having 14 carbon atoms (“C₁₄ aryl”), such as anthranyl. When the C₆₋₂₀ aryl is substituted, it can be monosubstituted or polysubstituted. Further, the substitution site is not limited, for example, may be ortho-, para-, or meta-substitution.

The term “5-14-membered heteroaryl” should be construed to comprise such a monovalent monocyclic, bicyclic, or tricyclic aromatic ring system that has 5, 6, 7, 8, 9, 10, 11, 12, 13, or 14 ring atoms, particularly 5 or 6 or 9 or 10 carbon atoms, and comprises from 1 to 5, preferably from 1 to 3, heteroatoms each independently selected from N, O, and S, and, further, may be additionally benzo-fused in each circumstance. “Heteroaryl” also means a radical in which a heteroaromatic ring is fused with one or more aryl, alicyclic, or heterocyclic rings, wherein the radical or site of attachment is on the heteroaromatic ring. Non-limiting examples comprise 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7- or 8-quinolinyl, 1-, 3-, 4-, 5-, 6-, 7- or 8-isoquinolinyl, 1-, 4-, 5-, 6-, 7- or 8-phthalazinyl, 2-, 3-, 4-, 5-, or 6-naphthyridinyl, 2-, 3-, 5-, 6-, 7- or 8-quinazolinyl, 3-, 4-, 5-, 6-, 7- or 8-cinnolinyl, 2-, 4-, 6-, or 7-pteridinyl, 1-, 2-, 3-, 4-, 5-, 6-, 7-, or 8-4aH carbazolyl, 1-, 2-, 3-, 4-, 5-, 6-, 7-, or 8-carbazolylcarbazolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-pyridyl, 2-, 3-, 4-, 5-, 6-, 8-, 9-, or 10-phenanthrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4- , 6-, 7-, 8-, 9-, or 10-phenazinyl, 2-, 3-, 4-, 5-, 6- or 1-, 3-, 4-, 5-, 6-, 7-, 8-, 9- or 10-benzoisoquinolyl, 2-, 3-, 4- or thieno[2,3-b]furanyl, 2-, 3-, 5-, 6-, 7-, 8-, 9-, 10- or 11-7H-pyrazino[2,3-c]carbazolyl, 2-, 3-, 5-, 6- or 7-2H-furo[3,2-b]pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 54H-imidazo[4,5-d]thiazolyl, 3-, 5-, or 8-pyrazino[2,3-d]pyridazinyl, 2-, 3-, 5-, or 6-imidazo[2,1-b]thiazolyl, 1-, 3-, 6-, 7-, 8-, or 9-furo[3,4-c]cinnolinyl, 1-, 2-, 3-, 4-, 5-, 6-, 8-, 9-, 10 or 11-4H-pyrido[2,3-c]carbazolyl, 2-, 3-, 6- or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 11-4H-pyrrolo[1,2-b][2]benzazepinyl. A typical fused heteroaryl includes, but is not limited to, 2-, 3-, 4-, 5-, 6-, 7-, or 8-quinolyl, 1-, 3-, 4-, 5-, 6-, 7-, or 8-isoquinolyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, and 2-, 4-, 5-, 6-, or 7-benzothiazolyl. The 5-20-membered heteroaryl may be connected with other radicals to form the compound of the present invention by connecting a carbon atom on a 5-20-membered heteroaryl ring with the other radicals, or by connecting a heteroatom on a 5-20-membered heteroaryl ring with the other radicals. When the 5-20-membered heteroaryl is substituted, it may be monosubstituted or polysubstituted. Further, the substitution site is not limited, for example, hydrogen attached to a carbon atom on the heteroaryl ring may be substituted, or hydrogen attached to a heteroatom on the heteroaryl ring may be substituted.

The term “spirocyclic ring” refers to a ring system in which two rings share 1 ring-forming atom.

The term “fused ring” refers to a ring system in which two rings share 2 ring atoms.

The term “bridged ring” refers to a ring system in which two rings share more than 3 ring-forming atoms.

Unless otherwise specified, the heterocyclyl, heteroaryl, or heteroarylene comprises all possible isomeric forms thereof, such as positional isomers thereof. Therefore, for some illustrative non-limiting examples, the substitution or bonding to other radicals may be comprised at, e.g., 1, 2, or more of its 1-, 2-, 3-, 4-, 5-, 6-, 7-, 8-, 9-, 10-, 11-, 12-C, (if present), comprising pyridin-2-yl, pyridyliden-2-yl, pyridin-3-yl, pyridyliden-3-yl, pyridin-4-yl, and pyridyliden-4-yl; and the thienyl or thienylenyl comprises thien-2-yl, thienylen-2-yl, thien-3-yl, and thienylen-3-yl; pyrazol-1-yl, pyrazol-3-yl, pyrazol-4-yl, and pyrazol-5-yl.

The term “alkoxy” refers to —O-(alkyl), wherein the alkyl is as defined above. Non-limiting examples of alkoxy comprise: methoxy, ethoxy, propoxy, and butoxy. The alkoxy may be optionally substituted or unsubstituted, and may be, when substituted, substituted with a substituent which is preferably one or more radicals independently selected from alkyl, alkenyl, alkynyl, alkoxy, alkylamino, halogen, sulfydryl, hydroxyl, nitro, cyano, cycloalkyl, heterocycloalkyl, aryl, heteroaryl, cycloalkoxy, or heterocycloalkoxy.

The term “alkylamino” refers to —NH-(alkyl), wherein the alkyl is as defined above. Non-limiting examples of alkylamino comprise, for example, methylamino, ethylamino, propylamino, isopropylamino, and butylamino.

The term “(alkyl)₂amino” refers to —N-(alkyl)₂, wherein the alkyl is as defined above. Non-limiting examples of (alkyl)₂amino comprise: for example, dimethylamino, methylethylamino, diethylamino, dipropylamino, methylpropylamino, diisopropylamino, and dibutylamino.

The “haloalkyl” refers to an alkyl substituted with one or more halogens, wherein the alkyl is as defined above.

The term peptide residue refers to an oligopeptide fragment in an ADC linker, which often serves as a substrate for enzymes in tumor cells or the microenvironment to control the release of a drug, such as GGFG, and has a structure of

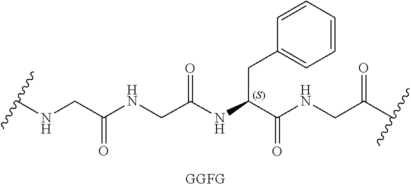

Those skilled in the art will understand that the compound represented by formula (I) may exist in the form of various pharmaceutically acceptable salts. If the compounds have a basic center, they can form acid addition salts; if they have an acidic center, they can form base addition salts; if the compounds contain both an acidic center (e.g., carboxyl) and a basic center (e.g., amino), they can also form inner salts.

The compounds of the present invention may be present in the form of solvates, such as hydrates, wherein the compounds of the present invention comprise polar solvents, such as water, methanol or ethanol, as structural elements of the lattice of the compounds. The polar solvent, especially water, may be present in a stoichiometric or non-stoichiometric amount.

Depending on their molecular structure, the compounds of the present invention may be chiral and may therefore exist in various enantiomeric forms. These compounds may therefore exist in racemic or optically active forms. The compounds of the present invention encompass isomers in which the chiral carbons are each in R or S configuration, or mixtures and racemates thereof. The compounds of the present invention or their intermediates can be separated into enantiomeric compounds by chemical or physical methods known to those skilled in the art, or used in synthesis in this form. In the case of racemic amines, diastereomers are prepared from the mixture by reaction with an optically active resolving reagent. Examples of suitable resolving reagents are optically active acids, such as the R and S forms of tartaric acid, diacetyltartaric acid, dibenzoyltartaric acid, mandelic acid, malic acid, lactic acid, appropriate N-protected amino acids (e.g., N-benzoylproline or N-phenylsulfonylproline) or various optically active camphorsulfonic acids. Chromatographic enantiomeric resolution can also be advantageously carried out by means of optically active resolving reagents, such as dinitrobenzoylphenylglycine, cellulose triacetate or other carbohydrate derivatives or chirally derivatized methacrylate polymers immobilized on silica gel. Suitable eluents for this purpose are aqueous or alcoholic solvent mixtures, for example, hexane/isopropanol/acetonitrile.

The corresponding stable isomers can be separated according to known methods, for example by extraction, filtration or column chromatography.

The term “patient” refers to any animal, comprising mammals, preferably mice, rats, other rodents, rabbits, dogs, cats, pigs, cows, sheep, horses or primates, and most preferably humans.

The term “therapeutically effective amount” refers to that amount of an active compound or drug which causes a biological or medical response sought by a researcher, veterinarian, physician or other clinician in a tissue, system, animal, individual or human, and which comprises one or more of the following: (1) prevention of disease: for example, prevention of a disease, disorder or condition in an individual who is susceptible to the disease, disorder or condition but has not yet experienced or developed the pathology or symptoms of the disease. (2) Inhibition of disease: for example, inhibition of a disease, disorder or condition (i.e., prevention of further progression of pathology and/or symptoms) in an individual undergoing or exhibiting the pathology or symptoms of the disease, disorder or condition. (3) Alleviation of disease: for example, alleviation of a disease, disorder or condition (i.e., reversion of pathology and/or symptoms) in an individual undergoing or exhibiting the pathology or symptoms of the disease, disorder or condition.

### DETAILED DESCRIPTION OF THE INVENTION

The technical solution of the present invention will be further described in detail below in conjunction with specific embodiments. It should be understood that the following embodiments are only intended to exemplify and explain the present invention and should not be construed as limiting the scope of protection of the present invention. All technologies realized based on the above contents of the present invention are comprised in the scope that the present invention intends to protect.

Unless otherwise stated, the raw materials and reagents used in the following examples are commercially available, or may be prepared in accordance with known methods.

The structures of the compounds are determined by nuclear magnetic resonance (NMR) or/and mass spectrum (MS). NMR shifts (6) are given in 10⁻⁶ (ppm). A nuclear magnetic resonance spectrometer (Bruker AVANCE-400) is used for NMR measurement with a solvent of deuterated dimethyl sulfoxide (DMSO-d₆), deuterated chloroform (CDCl₃), or deuterated methanol (CD₃OD), and with an internal standard of tetramethylsilane (TMS).

Agilent 1200/1290 DAD-6110/6120 Quadrupole MS/LC/MS (manufacturer: Agilent, MS model: 6110/6120 Quadrupole MS), waters ACQuity UPLC-QD/SQD (manufacturer: waters, MS model: waters ACQuity Qda Detector/waters SQ Detector), and THERMO Ultimate 3000-Q Exactive (manufacturer: THERMO, MS model: THERMO Q Exactive) are used for MS measurement.

Agilent 126011 HPLC and Waters Acquity UPLC H-Class high performance liquid chromatograph are used for high performance liquid chromatography (HPLC) analysis.

Waters Acquity UPCC high performance liquid chromatograph is used for chiral HPLC analysis and determination.

Waters MS-triggered Prep-LC with SQD2 detector, Waters MS triggered Prep-LC with Acquity QDA detector, Waters MS-triggered Prep-LC with QDA detector, and GILSON Prep LC with UV detector are used for preparative high performance liquid chromatography.

Combiflash Rf200 (TELEDYNE ISCO) is used for CombiFlash fast preparative chromatograph.

Yantai Huanghai HSGF254 or Qingdao GF254 silica gel plate is used as the silica gel plate for thin layer chromatography, the specification of the silica gel plate for thin layer chromatography (TLC) is from 0.15 mm to 0.2 mm, and the specification for thin layer chromatography for separation and purification of products is from 0.4 mm to 0.5 mm.

Yantai Huanghai silica gel of 200-300 mesh is generally used as the carrier for silica gel column chromatography.

The average inhibition rate and IC₅₀ value of kinases are determined using NovoStar microplate reader (German BMG).

Known starting materials of the present invention may be synthesized using or in accordance with methods known in the art, or may be purchased from, e.g., ABCR GmbH & Co. KG, Acros Organics, Aldrich Chemical Company, Accela ChemBio Inc, or Chembee Chemicals.

Unless otherwise specified in the examples, reactions can all be carried out in an argon atmosphere or a nitrogen atmosphere.

The argon atmosphere or the nitrogen atmosphere means that the reaction flask is connected to an argon or nitrogen balloon with a volume of about 1 L.

A hydrogen atmosphere means that the reaction flask is connected to a hydrogen balloon with a volume of about 1 L.

Parr 3916EKX hydrogenator and QL-500 hydrogen generator or HC2-SS hydrogenator are used for a pressurized hydrogenation reaction.

Vacuumization and filling with hydrogen are usually repeatedly operated 3 times prior to the hydrogenation reaction.

CEM Discover-S 908860 microwave reactor is used for a microwave reaction.

Unless otherwise specified in the examples, the solution refers to an aqueous solution.

Unless otherwise specified in the examples, the reaction temperature is room temperature (20° C.-30° C.).

Thin layer chromatography (TLC) is used for monitoring of reaction processes in the examples. A developing agent used in a reaction, an eluent system of column chromatography used for purifying a compound, and a developing agent system of the thin layer chromatography comprise: A: dichloromethane/methanol system, and B: n-hexane/ethyl acetate system, wherein a volume ratio of the solvent is adjusted based on polarity of the compound, or adjusted optionally by addition of a small amount of an alkaline or acidic reagent such as triethylamine or acetic acid.

### Example 1

### (R)—N-((1S,9S)-9-ethyl-5-fluoro-4,9-dihydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

### (R)—N-((1R,9S)-9-ethyl-5-fluoro-4,9-dihydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

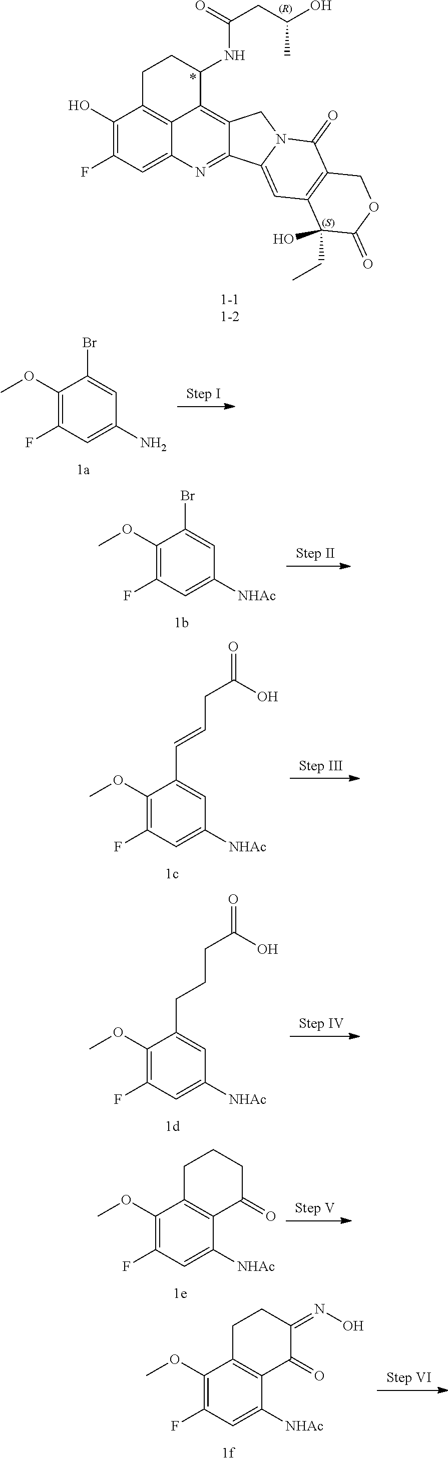

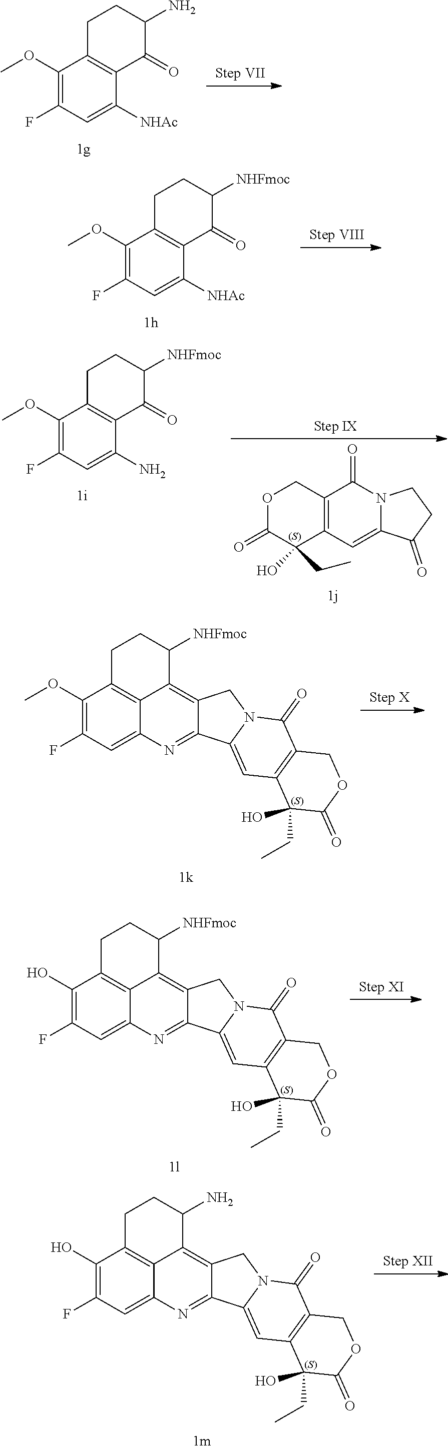

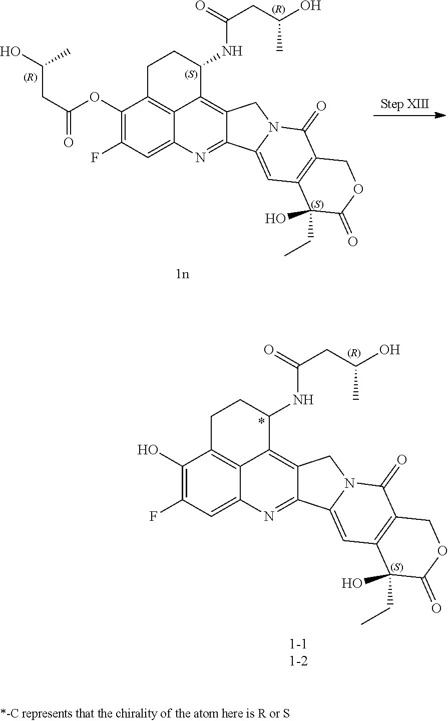

### Step I N-(3-bromo-5-fluoro-4-methoxyphenyl)acetamide 1b

3-bromo-5-fluoro-4-methoxyaniline 1a (20 g, 90.8 mmol) was dissolved in dichloromethane (40 mL). After the mixture was cooled to 0° C., acetyl chloride (14.3 g, 181.6 mmol) and triethylamine (27.6 mg, 272.4 mmol) were slowly added. The reaction mixture was stirred at 0° C. for 0.5 h. After the reaction was complete, water (30 mL) was added to the reaction mixture. The mixture was extracted with dichloromethane (50 mL×3). The organic phases were combined, washed with saturated brine, dried, and concentrated. The resulting residue was purified by silica gel column chromatography with an eluent system B, to give the title compound 1b (20 g, yield: 84%).

MS m/z (ESI): 262.1 (M+H)⁺.

### Step II (E)-4-(5-acetylamino-3-fluoro-2-methoxyphenyl)but-3-enoic acid 1c

The compound 1b (20 g, 76.3 mmol) was dissolved in dioxane (30 mL) and water (10 mL), to which but-3-enoic acid (7.23 g, 83.9 mmol), palladium acetate (1.71 g, 7.6 mmol), tris(o-methylphenyl)phosphorus (4.64 g, 15.2 mmol), and N,N-diisopropylethylamine (30 mg, 229 mmol) were added. The reaction mixture was stirred at 100° C. for 16 h. After the reaction was complete, the reaction mixture was filtered, and the resulting residue was purified by silica gel column chromatography with the eluent system B to give the title compound 1c (20 g, yield: 87%).

MS m/z (ESI): 268.1 (M+H)⁺.

### Step III 4-(5-acetylamino-3-fluoro-2-methoxyphenyl)butyric acid 1d

The compound 1c (20 g, 74.8 mmol) was dissolved in tetrahydrofuran (50 mL), and 10% Pd/C (0.8 g, 7.4 mmol) was added. The reaction mixture was stirred in a hydrogen atmosphere at room temperature for 2 h. After the reaction was complete, the reaction mixture was filtered, and the filtrate was concentrated under reduced pressure to give the crude product. The resulting residue was purified by silica gel column chromatography with an eluent system A to give the title compound 1d (20 g, yield: 99%).

MS m/z (ESI): 270.1 (M+1)⁺.

### Step IV N-(3-fluoro-4-methoxy-8-oxo-5,6,7,8-tetralin-1-yl)acetamide 1e

The compound 1d (20 g, 74.4 mmol) was dissolved in trifluoroacetic acid (60 mL), the mixture was cooled to 0° C., and then trifluoroacetic anhydride (31.24 g, 148.8 mmol) was slowly added. The reaction mixture was stirred at room temperature for 7 h. After the reaction was complete, the reaction mixture was slowly poured into water (50 mL), and extracted with ethyl acetate (50 mL×3). The organic phases were combined, washed with a saturated aqueous solution of sodium bicarbonate until neutral, then washed with saturated brine, dried, and concentrated. The resulting residue was purified by silica gel column chromatography with the eluent system B, to give the title compound 1e (9.2 g, yield: 46%).

MS m/z (ESI): 252.1 (M+1)⁺.

### Step V (Z)—N-(3-fluoro-7-(hydroxyimino)-4-methoxy-8-oxo-5,6,7,8-tetralin-1-yl)acetamide 1f

Potassium tert-butoxide (9.83 g, 87.5 mmol) was dissolved in tetrahydrofuran (40 mL) and tert-butanol (10 mL), and the mixture was cooled to 0° C. Then, N-(3-fluoro-4-methoxy-8-oxo-5,6,7,8-tetralin-1-yl)acetamide 1e (10 g, 39.8 mmol) was dissolved in tetrahydrofuran (10 mL), the resulting mixture was slowly added to the reaction mixture, and ten minutes later, isoamyl nitrite (7.46 g, 63.6 mmol) was added. The reaction mixture was stirred at 0° C. for 1 h. After the reaction was complete, a saturated ammonium chloride solution was added to the reaction mixture to quench the reaction. The mixture was extracted with ethyl acetate (50 mL×3). The organic phases were combined, then washed with saturated brine, dried, and concentrated. The resulting residue was purified by silica gel column chromatography with the eluent system B, to give the title compound 1f (6 g, yield: 51%).

MS m/z (ESI): 281.1 (M+1)⁺.

### Step VI N-(7-amino-3-fluoro-4-methoxy-8-oxo-5,6,7,8-tetralin-1-yl)acetamide 1g

(Z)—N-(3-fluoro-7-(hydroxyimino)-4-methoxy-8-oxo-5,6,7,8-tetralin-1-yl)acetamide 1f (6 g, 21.4 mmol) was dissolved in dioxane (60 mL) and 2 N hydrochloric acid solution (20 mL), and 10% Pd/C (1.13 g, 10.7 mmol) was added. The reaction mixture was stirred in a hydrogen atmosphere at room temperature for 5 h. After the reaction was complete, the reaction mixture was filtered and concentrated to give the crude product 1g (5 g), which was directly used in the next step without purification.

MS m/z (ESI): 267.1 (M+1)⁺.

### Step VII (9H-fluoren-9-yl)methyl(8-acetamido-6-fluoro-5-methoxy-1-oxo-1,2,3,4-tetralin-2-yl)carbamate 1h

The compound 1g (5 g, 18.8 mmol) obtained from the previous step was dissolved in dioxane (50 mL), and after the solution was adjusted with sodium bicarbonate to pH=7-8, 9-fluorenylmethyl-N-succinimidyl carbonate (6.36 g, 18.8 mmol) was slowly added. The reaction mixture was stirred at room temperature for 1 h. After the reaction was complete, the reaction mixture was slowly poured into water (40 mL), and extracted with ethyl acetate (40 mL×3). The organic phases were combined, washed with saturated brine, dried, and concentrated.

The resulting residue was purified by silica gel column chromatography with the eluent system B, to give the title compound 1h (5.3 g, yield: 54%).

MS m/z (ESI): 489.2 (M+1)⁺.

### Step VIII (9H-fluoren-9-yl)methyl(8-amino-6-fluoro-5-methoxy-1-oxo-1,2,3,4-tetralin-2-yl)carbamate 1i

The compound 1h (5 g, 10.25 mmol) was dissolved in dioxane (50 mL) and 12 N hydrochloric acid (10 mL). The reaction mixture was stirred at 60° C. for 2 h. After the reaction was complete, the reaction mixture was slowly poured into water (50 mL), and extracted with ethyl acetate (40 mL×3).

The organic phases were combined, washed with saturated brine, dried, and concentrated. The resulting residue was purified by silica gel column chromatography with the eluent system B, to give the title compound 1i (3.75 g, yield: 78%).

MS m/z (ESI): 447.2 (M+1)⁺.

### Step IX (9H-fluoren-9-yl)methyl((9S)-9-ethyl-5-fluoro-9-hydroxy-4-methoxy-10,13-dioxo-2,3,9,10,13,15-hexahydro-1H,12H-benzo[de]pyrano[3′,4′,6,7]indolizino[1,2-h]quinolin-1-yl)carbamate 1k

The compound 1i (3 g, 6.6 mmol) was dissolved in toluene (30 mL), and (S)-4-ethyl-4-hydroxy-7,8-dihydro-1H-pyranindolizine-3,6,10(4H)-trione 1j (2.61 g, 9.9 mmol) and p-toluenesulfonic acid (2.52 g, 13.2 mmol) were added. The reaction mixture was stirred at 110° C. for 5 h. After the reaction was complete, the mixture was extracted with ethyl acetate (30 mL-3). The organic phases were combined, washed with saturated brine, dried, and concentrated. The resulting residue was purified by silica gel column chromatography with the eluent system B, to give the title compound 1k (1.8 g, yield: 36%).

MS m/z (ESI): 674.2 (M+1)⁺.

### Step X (9H-fluoren-9-yl)methyl((9S)-9-ethyl-5-fluoro-4,9-dihydroxy-10,13-dioxo-2,3,9,10,13,15-hexahydro-1H,12H-benzo[de]pyrano[3′,4′,6,7]indolizino[1,2-b]quinolin-1-yl)carbamate 1l

The compound 1k (1.8 g, 2.7 mmol) was dissolved in 40% hydrobromic acid (40 mL). The reaction mixture was stirred at 100° C. for 2 h. After the reaction was complete, the reaction mixture was extracted with ethyl acetate (50 mL-3). The organic phases were combined, washed with saturated brine, dried, and concentrated. The resulting residue was purified by silica gel column chromatography with the eluent system B, to give the title compound 11(1.08 g, yield: 60%).

MS m/z (ESI): 660.2 (M+1)⁺.

### Step XI (9S)-1-amino-9-ethyl-5-fluoro-4,9-dihydroxy-1,2,3,9,12,15-hexahydro-10H,13H-benzo[de]pyrano[3′,4′,6,7]indolizino[1,2-b]quinoline-10,13-dione 1m

The compound 1l (500 mg, 0.7 mmol) was dissolved in N,N-dimethylformamide (5 mL), and diethylamine (166 mg, 2.3 mmol) was added. The reaction mixture was stirred at room temperature for 1 h. After the reaction was complete, the reaction mixture was spin-dried using an oil pump to remove diethylamine, thus giving the crude product 1m, which was slurried with ethyl acetate to give a solid. The solid was directly used in the next reaction step.

MS m/z (ESI): 438.1 (M+1)⁺.

### Step XII (1S,9S)-9-ethyl-5-fluoro-9-hydroxy-1-((R)-3-hydroxybutyrylamino)-10,13-dioxo-2,3,9,10,13,15-hexahydro-1H,12H-benzo[de]pyrano[3′,4′,6,7]indolizino[1,2-b]quinolin-4-yl-(R)-3-hydroxybutyrate 1n

The compound 1m (100 mg, 0.2 mmol) was dissolved in N,N-dimethylformamide (3 mL), and (R)-3-hydroxybutyric acid (36 mg, 0.34 mmol), 2-(7-azabenzotriazole)-N,N,N′,N′-tramethyluronium hexafluorophosphate (174 mg, 0.46 mmol), and N,N-diisopropylethylamine (88 mg, 0.68 mmol) were added. The reaction mixture was stirred at room temperature for 1 h. After the reaction was complete, the reaction mixture was extracted with ethyl acetate (50 mL×3). The organic phases were combined, washed with saturated brine, dried, and concentrated. The resulting residue was purified by silica gel column chromatography with the eluent system B, to give the title compound 1n (90 mg, yield: 64%).

MS m/z (ESI): 610.2 (M+1)⁺.

### Step XIII

### (R)—N-((1S,9S)-9-ethyl-5-fluoro-4,9-dihydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

### (R)—N-((1R,9S)-9-ethyl-5-fluoro-4,9-dihydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

The compound in (90 mg, 0.15 mmol) was dissolved in methanol (5 mL), and then an aqueous solution of lithium hydroxide (2 mL, 1 M) was added at room temperature. The mixture was stirred at room temperature for reaction for 15 min. After the reaction was complete, methanol was spun away, and the remaining aqueous phase was lyophilized to give the crude product, which was purified by preparative high performance liquid chromatography (Waters MS-triggered Prep-LC with SQD2 detector, chromatographic column: Xbridge C18 150*19 mm, 5 μm; mobile phase 1: water (containing 0.1% TFA); mobile phase 2: acetonitrile; 15-min gradient, gradient proportion: acetonitrile phase 23%-33%, flow rate: 20 mL/min) to give compound 1-1 (35 mg, yield: 30%) and compound 1-2 (28 mg, yield: 24%).

### Monoconfigurational Compound 1-1 (Short Retention Time):

MS m/z (ESI): 524.2 (M+1)⁺.

¹H NMR (400 MHz, DMSO-d₆) δ 10.42 (s, 1H), 8.39 (d, 1H), 7.84 (d, 1H), 7.26 (s, 1H), 6.50 (s, 1H), 5.57-5.49 (m, 1H), 5.41 (s, 2H), 5.27-5.11 (m, 2H), 4.04 (dd, 1H), 3.18-3.04 (m, 2H), 2.36-2.17 (m, 2H), 2.15-1.96 (m, 2H), 1.92-1.77 (m, 2H), 1.08 (d, 3H), 0.93-0.81 (m, 3H).

### Monoconfigurational Compound 1-2 (Long Retention Time):

MS m/z (ESI): 524.2 (M+1)⁺.

¹H NMR (400 MHz, DMSO-4) δ 10.42 (s, 1H), 8.42 (d, 1H), 7.84 (d, 1H), 7.26 (s, 1H), 5.59-5.51 (m, 1H), 5.42 (s, 2H), 5.29-5.14 (m, 2H), 4.04 (dd, 1H), 3.17 (dd, 1H), 3.10-2.92 (m, 1H), 2.28 (dd, 1H), 2.18 (dd, 1H), 2.13-1.95 (m, 2H), 1.93-1.78 (m, 2H), 1.08 (d, 3H), 0.91-0.82 (m, 3H).

### Example 2

### (R)—N-((1S,9S)-9-ethyl-4,9-dihydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

### (R)—N-((1R,9S)-9-ethyl-4,9-dihydroxy-10,13-dioxo-2,3,9,10,13,15-hexahydro-11H,12H-benzo[de]pyrano[3′,4′,6,7]indolizino[1,2-b]quinolin-1-yl)-3-hydroxybutanamide

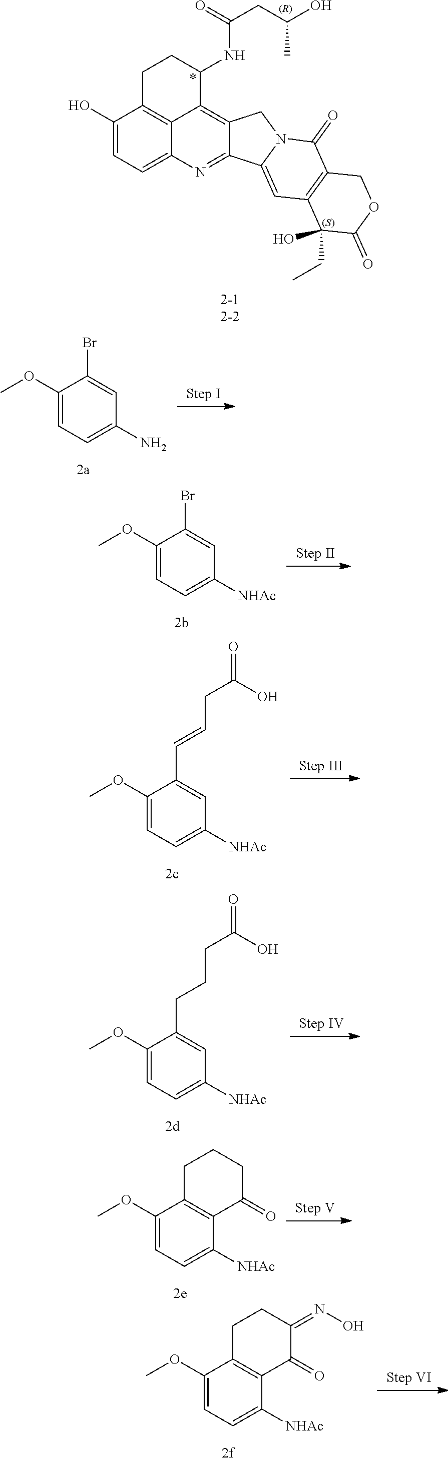

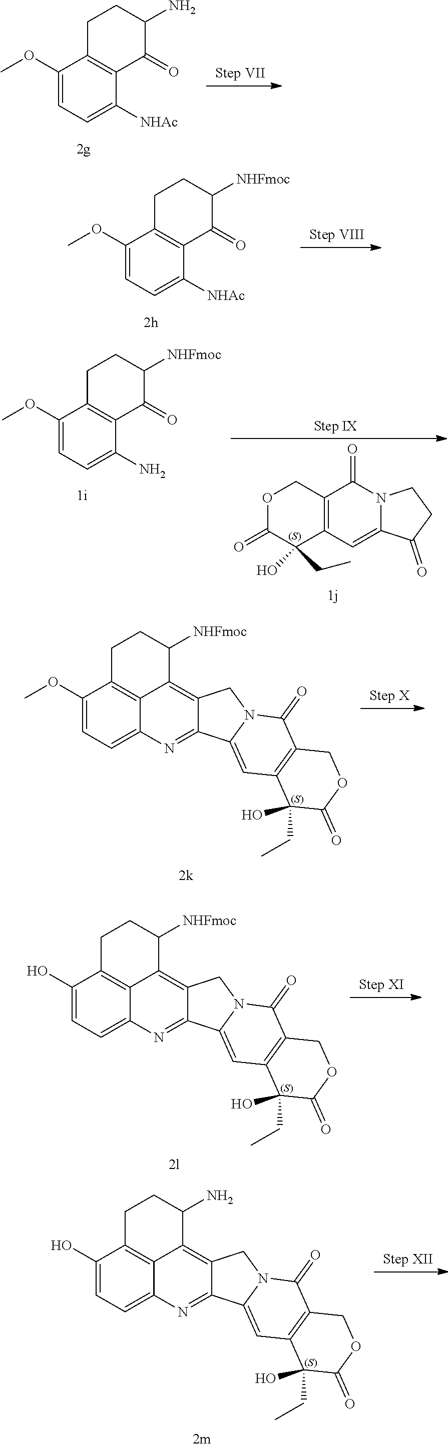

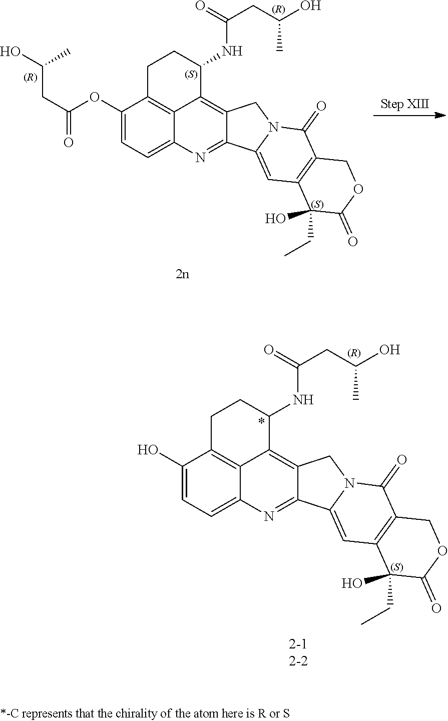

In accordance with the synthetic route in Example 1, the raw material from the step I was replaced with 3-bromo-4-methoxyaniline 2a (25 g, 0.12 mol), and the purification was performed by preparative high performance liquid chromatography (Waters MS-triggered Prep-LC with SQD2 detector, chromatographic column: Xbridge C18 150*19 mm, 5 μm; mobile phase 1: water (0.1% TFA); mobile phase 2: acetonitrile; 15-min gradient, gradient proportion: acetonitrile phase 23%-33%, flow rate: 20 mL/min) to give the captioned products 2-1 (19 mg, yield: 28%) and 2-2 (30 mg, yield: 44%).

### Monoconfigurational Compound 2-1 (Short Retention Time):

MS m/z (ESI): 506 (M+1)⁺.

¹H NMR (400 MHz, DMSO-d₆) δ 10.21 (s, 1H), 8.39-8.37 (m, 1H), 7.92-7.90 (m, 1H), 7.51-7.50 (m, 1H), 7.24 (s, 1H), 6.49 (s, 1H), 5.57-5.48 (m, 1H), 5.41 (s, 2H), 5.27-5.11 (m, 2H), 4.65 (s, 1H), 4.09-3.98 (m, 1H), 3.09-2.96 (m, 2H), 2.25-2.23 (m, 2H), 2.05-2.00 (m, 2H), 1.95-1.77 (m, 2H), 1.23 (s, 2H), 1.08-1.07 (m, 3H), 0.86-0.85 (m, 3H).

### Monoconfigurational Compound 2-2 (Long Retention Time):

MS m/z (ESI): 506 (M+1)⁺.

¹H NMR (400 MHz, DMSO-d₆) δ 10.22 (s, 1H), 8.42-8.40 (d, 1H), 7.93-7.90 (d, 1H), 7.51-7.50 (m, 1H), 7.25 (s, 1H), 5.58-5.46 (m, 1H), 5.42 (s, 2H), 5.21-5.20 (m, 2H), 4.04-4.01 (m, 1K), 2.96-2.94 (m, 2H), 2.28 (s, 2H), 2.10-1.97 (m, 2H), 1.86 (s, 2H), 1.23 (s, 2H), 1.08-1.06 (m, 3H), 0.87-0.85 (m, 3H).

### Example 3

### (R)—N-((1S′,9S)-4-bromo-9-ethyl-5-fluoro-9-hydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

(R)—N-((1R,9S′)-4-bromo-9-ethyl-5-fluoro-9-hydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

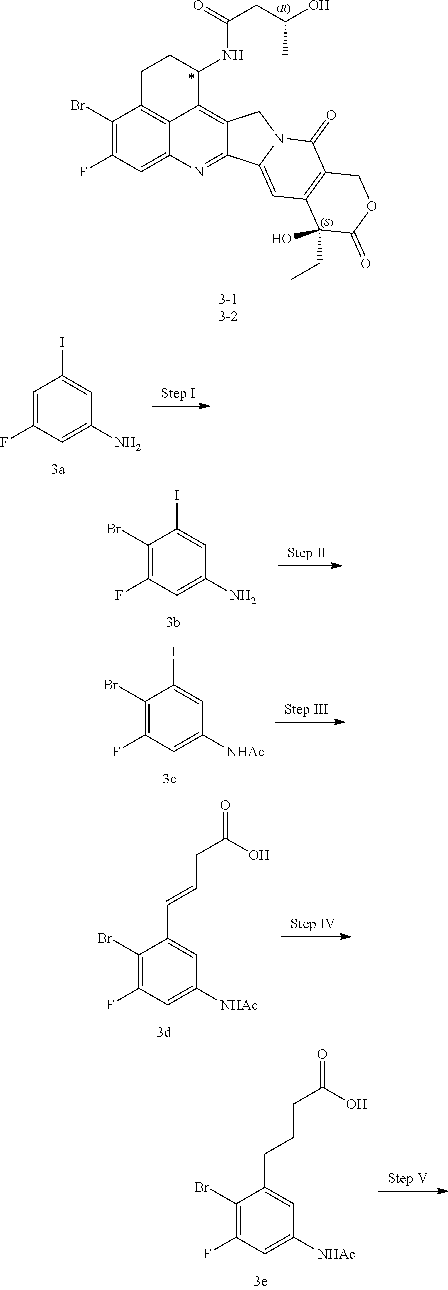

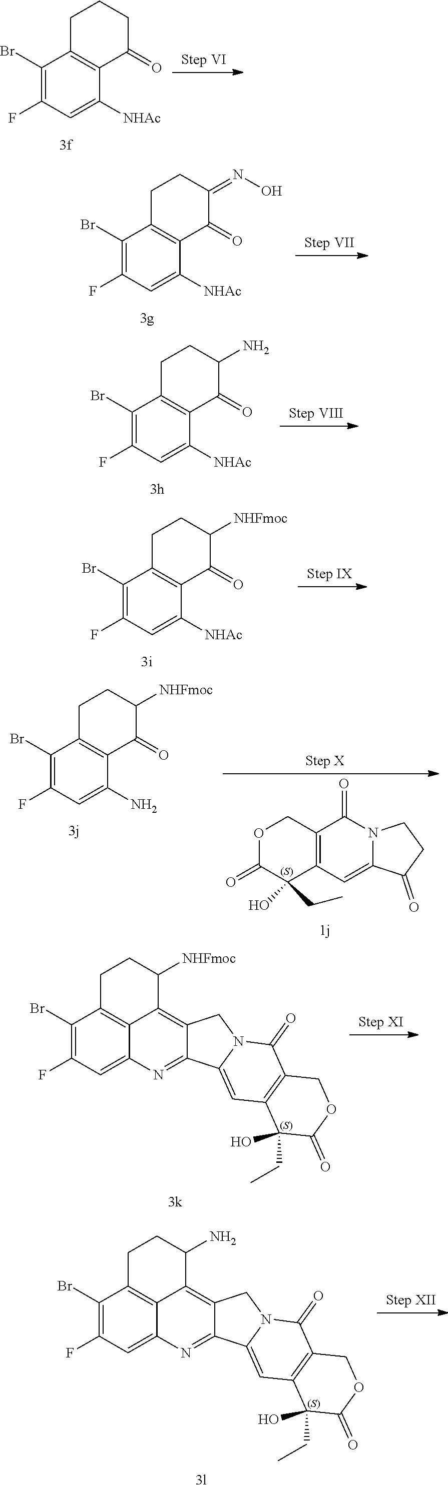

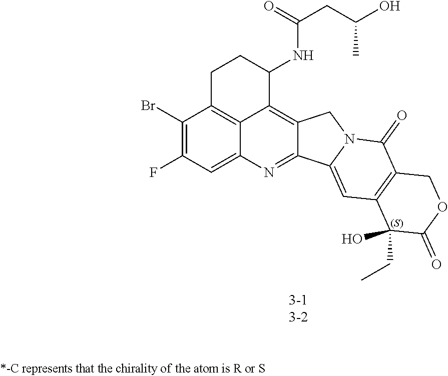

### Step I 4-bromo-3-fluoro-5-iodoaniline 3b

3-fluoro-5-iodoaniline 3a (50 g, 210.95 mmol) was dissolved in DMF (250 mL), and NBS (41.30 g, 232.04 mmol) was slowly added under an ice bath condition. The mixture was stirred for reaction at room temperature for 16 h. After the reaction was complete, water was added, and the system was extracted with dichloromethane. The organic phase was washed with saturated brine, collected, dried over anhydrous sodium sulfate, and spin-dried to remove the solvent, thus giving the crude product 3b (66 g).

MS m/z (ESI): 315.9, 317.9 (M+1)⁺.

¹H NMR (400 MHz, DMSO-d₆) δ 7.01 (d, 1H), 6.47 (dd, 1H), 5.74 (s, 2H).

### Step II N-(4-bromo-3-fluoro-5-iodophenyl)acetamide 3c

The compound 3b (66 g, 208.92 mmol) was dissolved in dichloromethane (660 mL), and triethylamine (42.28 g, 417.84 mmol) was added. After the system was cooled to 0° C., acetyl chloride (19.68 g, 250.70 mmol) was slowly added dropwise. After the dropwise addition was complete, the system was stirred at room temperature for 4 h. After the reaction was complete, the mixture was spin-dried to remove the solvent, thus giving the crude product, which was redissolved in ethyl acetate. Then, the system was adjusted with dilute hydrochloric acid to a pH of 2-3, then extracted with ethyl acetate, and washed with saturated brine. The organic phase was collected, dried over anhydrous sodium sulfate, and spin-dried to remove the solvent, thus giving the crude product, which was slurried with a mixed solvent of dichloromethane/methanol (10:1) to give the title compound 3c (60 g, yield: 80%).

MS m/z (ESI): 357.9, 359.9 (M+1)⁺.

¹H NMR (400 MHz, DMSO-d₆) δ 10.27 (s, 1H), 7.96 (s, 1H), 7.66 (dd, 1H), 2.06 (s, 3H).

### Step III (E)-4-(5-acetylamino-2-bromo-3-fluorophenyl)but-3-enoic acid 3d

The compound 3c (20 g, 55.87 mmol) was dissolved in a mixed solvent of dioxane (200 mL) and water (40 mL), and but-3-enoic acid (4.81 g, 55.87 mmol), DIPEA (14.45 g, 111.74 mmol), palladium acetate (630 mg, 2.79 mmol), and tris(o-methylphenyl)phosphorus (1.7 g, 5.59 mmol) were added. The mixture was stirred for reaction under the protection of nitrogen at 100° C. for 16 h. After the reaction was complete, water and dichloromethane were added, and the system was washed with a saturated sodium bicarbonate solution 3-5 times. The aqueous phase was collected, adjusted with hydrochloric acid to a pH of 2-3, and extracted with ethyl acetate 5-7 times. The organic phase was collected, dried over anhydrous sodium sulfate, and spin-dried to remove the solvent, thus giving the crude compound 3d (17 g).

MS m/z (ESI): 316.0, 317.9 (M+1)⁺.

### Step IV 4-(5-acetylamino-2-bromo-3-fluorophenyl)butyric acid 3e

The compound 3d (8 g, 25.3 mmol) was dissolved in methanol (80 mL), and a platinum-carbon catalyst (800 mg) was added. The mixture was stirred for reaction in a hydrogen environment at room temperature for 2 h. After the reaction was complete, the mixture was filtered. The filtrate was collected, and spin-dried to remove the solvent, thus giving the crude product 3e (8 g), which was directly used in the next reaction step without purification.

MS m/z (ESI): 318.0, 320.0 (M+1)⁺.

¹H NMR (400 MHz, DMSO-d₆) δ 12.14 (s, 1H), 10.24 (s, 1H), 7.63 (dd, 1H), 7.25 (s, 1H), 2.75-2.67 (m, 2H), 2.29 (t, 2H), 2.05 (s, 3H), 1.79 (dd, 2H).

### Step V N-(4-bromo-3-fluoro-8-oxo-5,6,7,8-tetralin-1-yl)acetamide 3f

The compound 3e (8 g, 25.1 mmol) was dissolved in trifluoroacetic acid (80 mL), and trifluoroacetic anhydride (15.82 g, 75.3 mmol) was slowly added under an ice bath condition. The mixture was stirred for reaction at 0° C. for 4 h. After the reaction was complete, water was added in an ice bath. The system was adjusted with 15% sodium hydroxide solution to a pH of 9-10, and then extracted with dichloromethane. The organic phase was washed with saturated brine, collected, dried over anhydrous sodium sulfate, and spin-dried to remove the solvent, thus giving the compound 3f (4.8 g, yield: 53%).

MS m/z (ESI): 329.0, 331.0 (M+1)⁺.

### Step VI (Z)—N-(4-bromo-3-fluoro-7-(hydroxyimino)-8-oxo-5,6,7,8-tetralin-1-yl)acetamide 3g

Potassium tert-butoxide (1.62 g, 14.4 mmol) was dissolved in a mixed solvent of tetrahydrofuran (80 mL) and tert-butanol (20 mL), and a solution of the compound 3f (2.15 g, 7.2 mmol) in tetrahydrofuran (20 mL) was slowly added in an ice bath. After the mixture was stirred for reaction at 0° C. for 10 min, isoamyl nitrite (1.27 g, 10.8 mmol) was added, and the reaction was continued by stirring at 0° C. for 50 min. After the reaction was complete, the system was adjusted with dilute hydrochloric acid to a pH of 4-5, and extracted with ethyl acetate. The organic phase was washed with saturated brine, collected, dried over anhydrous sodium sulfate, and spin-dried to remove the solvent, thus giving the crude product, which was slurried with methyl tert-butyl ether to give the title compound 3g (1.1 g, yield: 46%).

MS m/z (ESI): 329.0, 331.0 (M+1)⁺.

¹H NMR (400 MHz, CD₃OD) δ 8.52 (d, 1H), 3.21 (dd, 2H), 3.07 (dd, 2H), 2.24 (s, 3H).

### Step VII N-(7-amino-4-bromo-3-fluoro-8-oxo-5,6,7,8-tetralin-1-yl)acetamide 3h

The compound 3g (500 mg, 1.52 mmol) was dissolved in dioxane (10 mL), and 1 mL of 1M hydrochloric acid and a platinum-carbon catalyst (100 mg) were added. The mixture was stirred for reaction in a hydrogen environment at room temperature for 4 h. After the reaction was complete, the mixture was filtered. The filtrate was collected and concentrated to give the crude product 3h (500 mg), which was directly used in the next reaction step.

MS m/z (ESI): 315.1, 317.1 (M+1)⁺.

### Step VIII (9H-fluoren-9-yl)methyl(8-acetylamino-5-bromo-6-fluoro-1-oxo-1,2,3,4-tetralin-2-yl)carbamate 3i

The compound 3h (500 mg) was dissolved in dioxane (10 mL), the filtrate from the step VII was adjusted with a saturated sodium carbonate solution to a pH of 8-9, and then fluorenylmethoxycarbonylchloride (432 mg, 1.67 mmol) was added. The mixture was stirred for reaction at room temperature for 1 h. After the reaction was complete, the system was extracted with ethyl acetate. The organic phase was washed with saturated brine, collected, dried over anhydrous sodium sulfate, and spin-dried to remove the solvent, thus giving the crude product. The resulting residue was purified by silica gel column chromatography with the eluent system B, to give the title compound 3 (300 mg, yield: 37%).

MS m/z (ESI): 537.0, 539.0 (M+1)⁺.

### Step IX (9H-fluoren-9-yl)methyl(8-amino-5-bromo-6-fluoro-1-oxo-1,2,3,4-tetralin-2-yl)carbamate 3j

The compound 3i (700 mg, 1.30 mmol) was dissolved in methanol (10 mL), and concentrated hydrochloric acid (12 mol/L, 2 mL) was added. The mixture was stirred for reaction at 60° C. for 1 h. After the reaction was complete, the resulting residue was purified by silica gel column chromatography with the eluent system B, to give the title compound 3j (500 mg, yield: 77%).

### Step X ((9H-fluoren-9-yl)methyl((9S)-4-bromo-9-ethyl-5-fluoro-9-hydroxy-10,13-dioxo-2,3,9,10,13,15-hexahydro-1H,12H-benzo[de]pyrano[3′,4′,6,7]indolizino[1,2-h]quinolin-1-yl)carbamate 3k

The compound 3j (200 mg, 0.40 mmol) was dissolved in toluene (5 mL), and (S)-4-ethyl-4-hydroxy-7,8-dihydro-1H-pyrano[3,4-]indolizine-3,6,10(4H)-trione 1j (117 mg, 0.44 mmol) and p-toluenesulfonic acid monohydrate (77 mg, 0.40 mmol) were added. The mixture was stirred for reaction at 120° C. for 2 h. After the reaction was complete, the mixture was spin-dried to remove the solvent, thus giving the crude product. The resulting residue was purified by silica gel column chromatography with the eluent system B to give the title compound 3k (250 mg, yield: 86%).

MS m/z (ESI): 722.0, 724.0 (M+1)⁺.

¹H NMR (400 MHz, CDCl₃) δ 8.19-7.31 (m, 9H), 5.68 (dd, 1H), 5.33-5.18 (m, 2H), 4.66 (s, 2H), 4.34 (d, 1H), 4.21-4.04 (m, 2H), 3.26 (s, 1H), 3.00-2.94 (m, 1H), 2.04 (s, 2H), 1.81 (s, 2H), 1.70-1.50 (m, 2H), 1.27 (dd, 3H).

### Step XI (9S)-1-amino-4-bromo-9-ethyl-5-fluoro-9-hydroxy-1,2,3,9,12,15-hexahydro-10H,13H-benzo[de]pyrano[3′,4′,6,7]indolizino[1,2-b]quinoline-10,13-dione 3l

The compound 3k (200 mg, 0.28 mmol) was dissolved in DMF (1 mL), and diethylamine (0.1 mL) was added. The mixture was stirred for reaction at room temperature for 30 min. After the reaction was complete, the resulting residue was purified by silica gel column chromatography with the eluent system A, to give the title compound 31(100 mg, yield: 72%).

MS m/z (ESI): 500.0, 502.1 (M+1)⁺.

### Step XII

### (R)—N-((1S,9S)-4-bromo-9-ethyl-5-fluoro-9-hydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

### (R)—N-((1R,9S)-4-bromo-9-ethyl-5-fluoro-9-hydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

The compound 31(100 mg, 0.20 mmoL) was dissolved in DMF (2 mL), and (R)-3-hydroxybutyric acid (25 mg, 0.24 mmol), 2-(7-azobenzotriazole)-N,N,N′,N′-tramethyluronium hexafluorophosphate (114 mg, 0.30 mmol), and N,N-diisopropylethylamine (52 mg, 0.40 mmol) were added. The mixture was stirred for reaction at room temperature for 1 h. After the reaction was complete, the purification was performed by preparative high performance liquid chromatography (Waters MS-triggered Prep-LC with QDA detector, chromatographic column: WELCH Xtimate C18 21.2*250 mm, 10 μm; mobile phase 1: water (0.1% FA); mobile phase 2: acetonitrile; 15-min gradient, gradient proportion: acetonitrile phase 38%-48%, flow rate: 25 mL/min) to give compound 3-1 (1.62 mg, yield: 6%) and compound 3-2 (1.32 mg, yield: 7.4%).

### Monoconfigurational Compound 3-1 (Short Retention Time):

MS m/z (ESI): 586.0, 588.0 (M+1)⁺.

¹H NMR (400 MHz, CD₃OD) δ 8.10 (d, 1H), 7.90 (s, 1H), 5.96-5.88 (m, 1H), 5.83 (d, 1H), 5.71-5.48 (m, 4H), 4.5-24.46 (m, 1H), 2.71-2.65 (m, 2H), 2.59-2.52 (m, 2H), 2.23-2.18 (m, 3H), 1.49 (d, 3H), 1.25 (t, 3H).

### Monoconfigurational Compound 3-2 (Long Retention Time):

MS m/z (ESI): 586.0, 588.0 (M+1)⁺.

¹H NMR (400 MHz, CD₃OD) δ 7.79 (d, 1H), 7.59 (s, 1H), 5.67-5.59 (m, 1H), 5.52 (d, 1H), 5.41-5.33 (m, 2H), 5.32-5.23 (m, 2H), 4.24-4.18 (m, 1H), 2.34 (d, 2H), 2.28-2.21 (m, 2H), 1.93-1.86 (m, 3H), 1.17 (d, 3H), 0.94 (t, 3H).

### Example 4

### (R)—N-((1S,9S)-9-ethyl-5-fluoro-9-hydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

### (R)—N-((1R,9S)-9-ethyl-S-fluoro-9-hydroxy-10,13-dioxo-2,3,9,10,13,15-hexahydro-1H, 12H-benzo[de]pyrano[3′,4′,6,7]indolizino[1,2-b]quinolin-1-yl)-3-hydroxybutanamide

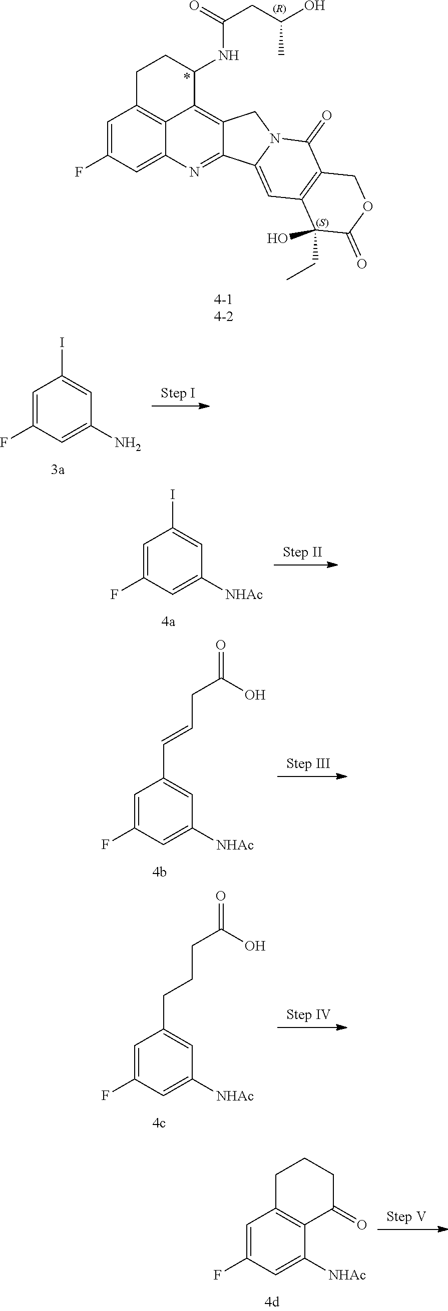

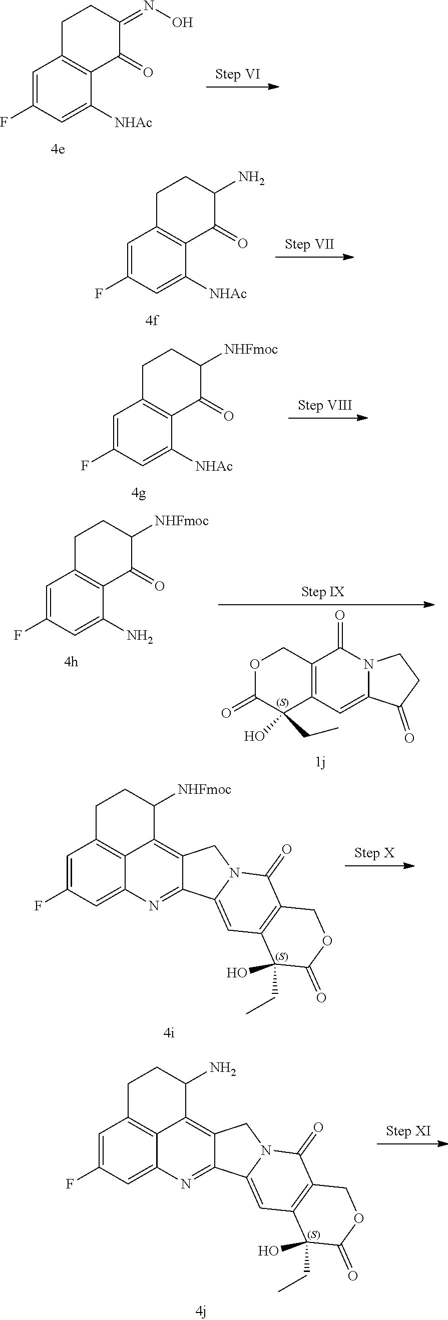

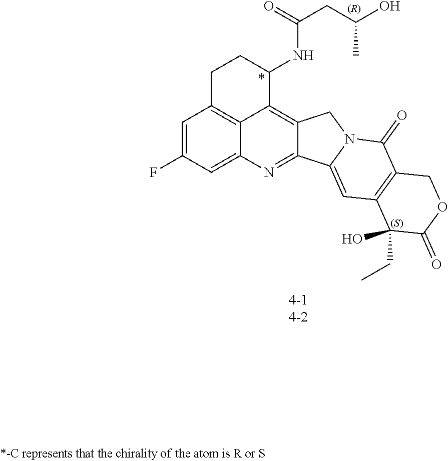

In accordance with the synthetic route in Example 3, 3-fluoro-5-iodoaniline (25 g, 105.5 mmol) was used as the starting material, and the purification was performed by preparative high performance liquid chromatography (Waters MS-triggered Prep-LC with QDA detector, chromatographic column: WELCH Xtimate C18 21.2*250 mm, 10 μm; mobile phase 1: water (0.1% FA); mobile phase 2: acetonitrile; 15-min gradient, gradient proportion: acetonitrile phase 38%-48%, flow rate: 25 mL/min) to give the captioned products 4-1 (2.5 mg, yield: 12%) and 4-2 (3.1 mg, yield: 15%).

### Monoconfigurational Compound 4-1 (Short Retention Time):

MS m/z (ESI): 508.1 (M+1)⁺.

¹H NMR (400 MHz, CD₃OD) δ 7.65 (d, 2H), 7.37 (d, 1H), 5.66 (s, 1H), 5.56 (d, 1H), 5.40-5.20 (m, 4H), 4.28-4.21 (m, 1H), 2.47-2.39 (m, 2H), 2.27 (s, 2H), 1.98-1.90 (m, 2H), 1.24 (d, 4H), 0.99 (t, 3H).

### Monoconfigurational Compound 4-2 (Long Retention Time):

MS m/z (ESI): 508.1 (M+1)⁺.

¹H NMR (400 MHz, CD₃OD) δ 7.57 (d, 2H), 7.30 (d, 1H), 5.60 (d, 1H), 5.48 (d, 1H), 5.36-5.15 (m, 5H), 4.22-4.15 (m, 1H), 2.36-2.30 (m, 2H), 2.18 (t, 3H), 1.90-1.83 (m, 2H), 1.15 (d, 3H), 0.91 (t, 3H).

### Example 5

### (R)—N-((1S,9S)-4-(difluoromethoxy)-9-ethyl-5-fluoro-9-hydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

### (R)—N-((1R,9S)-4-(difluoromethoxy)-9-ethyl-5-fluoro-9-hydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

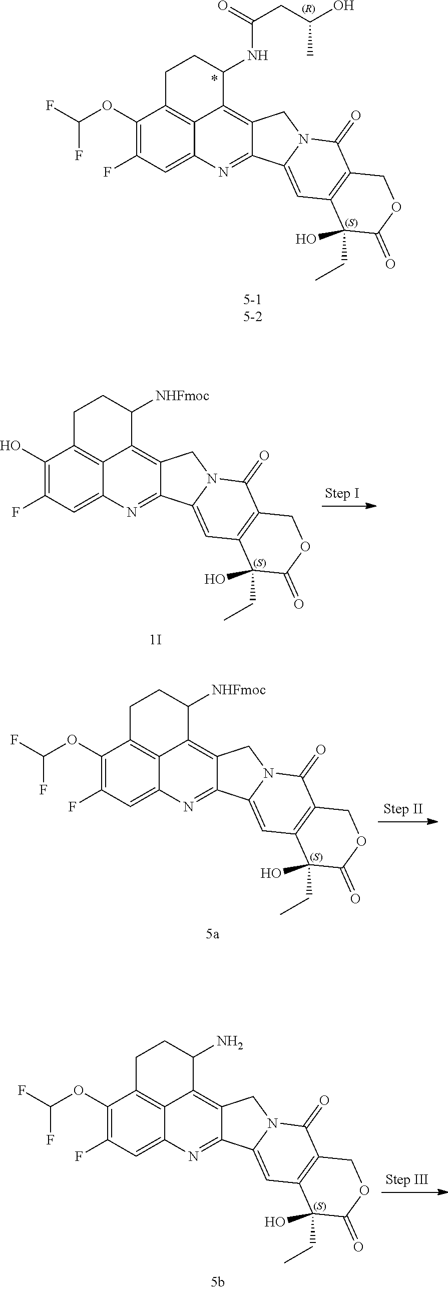

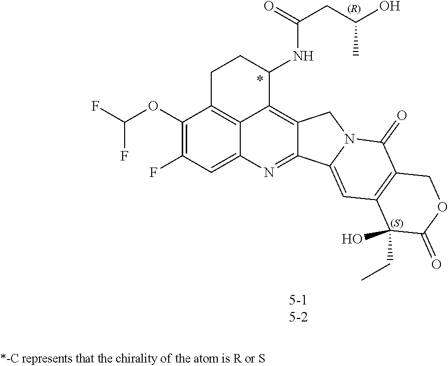

### Step I (9H-fluoren-9-yl)methyl((9S)-4-(difluoromethoxy)-9-ethyl-5-fluoro-9-hydroxy-10,13-dioxo-2,3,9,10,13,15-hexahydro-1H,12H-benzo[de]pyrano[3′,4′,6,7]indolizino[1,2-b]quinolin-1-yl)carbamate 5a

The compound 11(200 mg, 0.30 mmol) was dissolved in DMF (5 mL), and cesium carbonate (198 mg, 0.61 mmol) and diethyl (bromodifluoromethyl)phosphonate (121 mg, 0.45 mmol) were added in an ice bath. The mixture was stirred for reaction at 0° C. for 1 h. After the reaction was complete, water was added, and the system was extracted with ethyl acetate. The organic phase was washed with saturated brine, collected, dried over anhydrous sodium sulfate, and spin-dried to remove the solvent, thus giving the crude product, which was separated and purified by column chromatography to give the title compound 5a (200 mg, yield: 93%).

MS m/z (ESI): 711.2 (M+1)⁺.

### Step II (9S)-1-amino-4-(difluoromethoxy)-9-ethyl-5-fluoro-9-hydroxy-1,2,3,9,12,15-hexahydro-10H,13H-benzo[de]pyrano[3′,4′,6,7]indolizino[1,2-b]quinoline-10,13-dione 5b

The compound 5a (140 mg, 0.20 mmol) was dissolved in N,N-dimethylformamide (1 mL), and diethylamine (0.1 mL) was added. The mixture was stirred for reaction at room temperature for 1 h. After the reaction was complete, the reaction mixture was spin-dried under vacuum using an oil pump to remove the solvent, thus giving the crude title compound 5b (90 mg), which was directly used in the next reaction step without purification.

MS m/z (ESI): 488.1 (M+1)⁺.

Step III

### (R)—N-((1S,9S)-4-(difluoromethoxy)-9-ethyl-5-fluoro-9-hydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

### (R)—N—((1R,9S)-4-(difluoromethoxy)-9-ethyl-5-fluoro-9-hydroxy-10,13-dioxo-2,3,9,10,13,15-hexahydro-1H,12H-benzo[de]pyrano[3′,4′,6,7]indolizino[1,2-b]quinolin-1-yl)-3-hydroxybutanamide

The crude product 5b (90 mg, 0.18 mmol) was dissolved in N,N-dimethylformamide (1 mL), and (R)-3-hydroxybutyric acid (23 mg, 0.22 mmol), 2-(7-azobenzotriazole)-N,N,N′,N′-tramethyluronium hexafluorophosphate (105 mg, 0.28 mmol), and N,N-diisopropylethylamine (48 mg, 0.37 mmol) were added. The mixture was stirred for reaction at room temperature for 1 h. After the reaction was complete, the mixture was spin-dried to remove the solvent, thus giving the crude product, which was purified by preparative high performance liquid chromatography (GILSON Prep LC with UV detector, chromatographic column: Xtimate C18 250*30 mm, 10 μm; mobile phase 1: water (0.1% FA); mobile phase 2: acetonitrile; 15-min gradient, gradient proportion: acetonitrile phase 37%-47%, flow rate: 50 mL/min) to give the title compound 5-1 (8.6 mg, yield: 16%) and the title compound 5-2 (7.8 mg, yield: 14.7%).

### Monoconfigurational Compound 5-1 (Short Retention Time):

MS m/z (ESI): 574.1 (M+1)⁺.

¹H NMR (400 MHz, DMSO-d₆) δ 8.49 (d, 2H), 8.06 (d, 1H), 7.34 (s, 1H), 7.31 (t, 1H) 6.54 (s, 1H), 5.63-5.54 (m, 1H), 5.43 (s, 2H), 5.32-5.16 (m, 2H), 4.68 (d, 1H), 4.05 (s, 1H), 3.23 (d, 1H), 2.36-2.19 (m, 2H), 2.19-2.05 (m, 2H), 1.93-1.78 (m, 2H), 1.10 (d, 3H), 0.88 (t, 3H).

### Monoconfigurational Compound 5-2 (Long Retention Time):

MS m/z (ESI): 574.1 (M+1)⁺.

¹H NMR (400 MHz, DMSO-d₆) δ 8.52 (d, 2H), 8.10-8.04 (m, 1H), 7.35 (s, 1H), 7.32 (t, 1H) 6.55 (s, 11H), 5.66-5.58 (m, 1H), 5.43 (d, 2H), 5.26 (t, 2H), 4.66 (d, 11H), 4.14-4.01 (m, 1H), 3.25 (s, 11H), 2.34-2.20 (m, 2H), 2.16-2.07 (m, 2H), 1.92-1.80 (m, 2H), 1.10 (d, 3H), 0.89 (t, 3H).

### Example 6

### (R)—N—((1S,10S)-10-ethyl-6-fluoro-10-hydroxy-5-methyl-11,14-dioxo-1,2,3,4,10,11,14,16-octahydro-13H-cyclohepta[3′,4′,6,7]indolizino[1,2-b]quinolin-1-yl)-3-hydroxybutanamide

### (R)—N-((1R,10S)-10-ethyl-6-fluoro-10-hydroxy-5-methyl-11,14-dioxo-1,2,3,4,10,11,14,16-octahydro-13H-cyclohepta[3′,4′,6,7]indolizino[1,2-b]quinolin-1-yl)-3-hydroxybutanamide

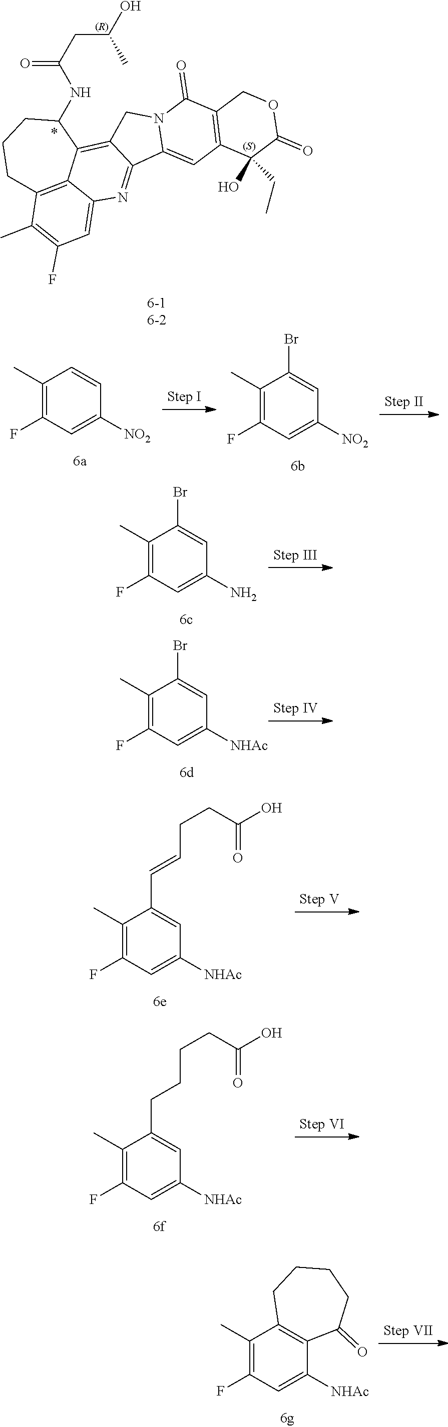

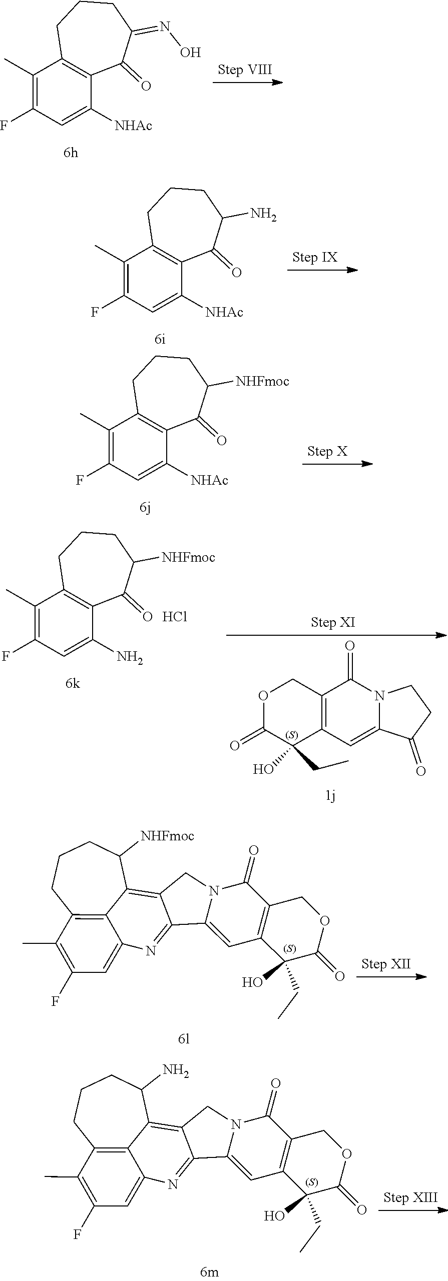

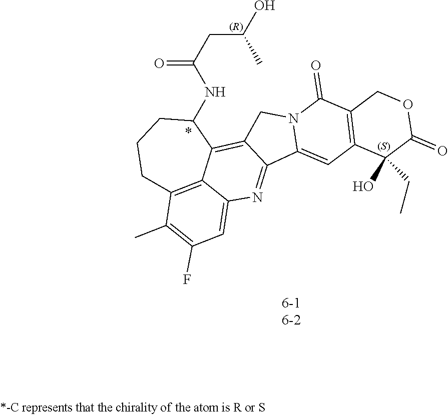

### Step I 1-bromo-3-fluoro-2-methyl-5-nitrobenzene 6b

2-fluoro-1-methyl-4-nitrobenzene 6a (20.0 g, 0.13 mol) was dissolved in n-heptane (50 mL), concentrated sulfuric acid (50 mL) was added, the mixture was heated to 60° C., N-bromosuccinimide (35.6 g, 0.20 mol) was added batchwise at this temperature, and the mixture was kept for reaction at 60° C. for 2 h. The reaction mixture cooled to room temperature was added dropwise to ice water, and extracted with toluene. The organic phases were combined, successively washed with a sodium sulfite solution, water, and saturated brine, dried over anhydrous sodium sulfate, and then concentrated under reduced pressure, to give the title compound 6b (30.0 g), which was directly used in the next reaction step without purification.

MS m/z (ESI): 233.9 (M+1)⁺.

¹H NMR (400 MHz, CDCl3) δ 8.29-8.23 (m, JH), 7.88 (dd, 1H), 2.44 (d, 3H).

### Step II 3-bromo-5-fluoro-4-methylaniline 6c

The compound 6b (30.0 g, 0.13 mol) was dissolved in methanol (200 mL), platinum-carbon (3.0 g, 5% content) was added, and the mixture was, after hydrogen replacement, kept for reaction in a hydrogen atmosphere at room temperature for 16 h. After the reaction was complete, the reaction mixture was filtered, and the filtrate was concentrated to give the title compound 6c (20.0 g, yellow oil), which was directly used in the next reaction step without purification.

MS m/z (ESI): 204.0 (M+1)⁺.

### Step III N-(3-bromo-5-fluoro-4-methylphenyl)acetamide 6d

3-bromo-5-fluoro-4-methylaniline 6c (20.0 g, 0.10 mol) was dissolved in dichloromethane (100 mL) under an ice bath condition, triethylamine (20.2 g, 0.20 mol) and acetyl chloride (11.8 g, 0.15 mol) were added, and the mixture was kept for reaction under an ice bath condition for 3 h. After the reaction was complete, water was added to the reaction mixture to quench the reaction, and the mixture was extracted with ethyl acetate. The organic phases were combined, dried over anhydrous sodium sulfate, and concentrated under reduced pressure to give the crude product, which was slurried with a mixed solvent of dichloromethane and petroleum ether (V/V=10:1) to give the title compound 6d (10.0 g, yield: 32%).

MS m/z (ESI): 246.0 (M+1)⁺.

### Step IV (E)-5-(5-acetylamino-3-fluoro-2-methylphenyl)pent-4-enoic acid 6e

N-(3-bromo-5-fluoro-4-methylphenyl)acetamide 6d (10.0 g, 40.8 mmol) was dissolved in dioxane (40 mL) and water (10 mL), to which pent-4-enoic acid (6.1 g, 61.20 mmol), palladium acetate (0.7 g, 4.10 mmol), tris(o-methylphenyl)phosphorus (2.5 g, 8.20 mmol), and N,N-diisopropylethylamine (15.9 g, 122.01 mmol) were added. The reaction mixture was stirred for reaction at 100° C. for 16 h. After the reaction was complete, water and dichloromethane were added, and the system was washed with a saturated sodium bicarbonate solution 3 times. The aqueous phase was collected, adjusted with hydrochloric acid to a pH of 2-3, and extracted with ethyl acetate 3 times. The organic phase was collected, dried over anhydrous sodium sulfate, and spin-dried to remove the solvent, thus giving the title compound 6e (10 g), which was directly used in the next reaction step without purification.

MS m/z (ESI): 266.1 (M+1)⁺.

The synthetic route in Example 3 was used as the subsequent synthetic route, except that the intermediate 3d in Example 3 was replaced with the intermediate 6e (10 g, 0.04 mol). The final product was purified by preparative high performance liquid chromatography (Waters MS-triggered Prep-LC with SQD2 detector, chromatographic column: Xbridge C18 150*19 mm, 5 μm; mobile phase 1: water (0.1% TFA); mobile phase 2: acetonitrile; 15-min gradient, gradient proportion: acetonitrile phase 37%47%, flow rate: 20 mL/min) to give the captioned products 6-1 (12.6 mg, yield: 14%) and 6-2 (17.2 mg, yield: 19%).

### Monoconfigurational Compound 6-1 (Short Retention Time):

MS m/z (ESI): 536 (M+1)⁺.

¹H NMR (400 MHz, DMSO-d₆) δ 8.60-8.55 (m, 1H), 7.73-7.72 (m, 1H), 7.28 (s, 1H), 6.51 (s, 1H), 5.54-5.53 (m, 1H), 5.42 (s, 2H), 5.32-5.26 (m, 1H), 5.24-5.22 (m, 1H), 4.65 (s, 1H), 4.01 (s, 1H), 3.22-3.18 (m, 2H), 2.42-2.40 (m, 3H), 2.28-2.24 (m, 4H), 2.10-1.67 (m, 4H), 1.06 (d, 3H), 0.85-0.82 (m, 3H).

### Monoconfigurational Compound 6-2 (Long Retention Time):

MS m/z (ESI): 536 (M+1)⁺.

¹H NMR (400 MHz, DMSO-d₆) δ 8.67-8.66 (m, 1H), 7.73-7.71 (m, 1H), 7.28 (s, 1H), 6.49 (s, 1H), 5.53-5.52 (m, 1H), 5.41-5.40 (m, 3H), 5.35-5.33 (m, 1H), 4.67-4.65 (m, 1H), 4.03-3.96 (m, 1H), 3.22-3.18 (m, 2H), 2.42 (s, 3H), 2.40-2.15 (m, 4H), 2.11-1.52 (m, 4H), 1.08-1.07 (m, 3H), 0.87-0.85 (m, 3H).

### Example 7

### (R)—N-((1S,9S)-9-ethyl-5-fluoro-9-hydroxy-10,13-dioxo-4-ethenyl-2,3,9,10,13,15-hexahydro-1H,12H-benzo[de]pyrano[3′,4′,6,7]indolizino[1,2-b]quinolin-1-yl)-3-hydroxybutanamide

### (R)—N-((1R,9S)-9-ethyl-5-fluoro-9-hydroxy-10,13-dioxo-4-ethenyl-2,3,9,10,13,15-hexahydro-1H,12H-benzo[de]pyrano[3′,4′,6,7]indolizino[1,2-b]quinolin-1-yl)-3-hydroxybutanamide

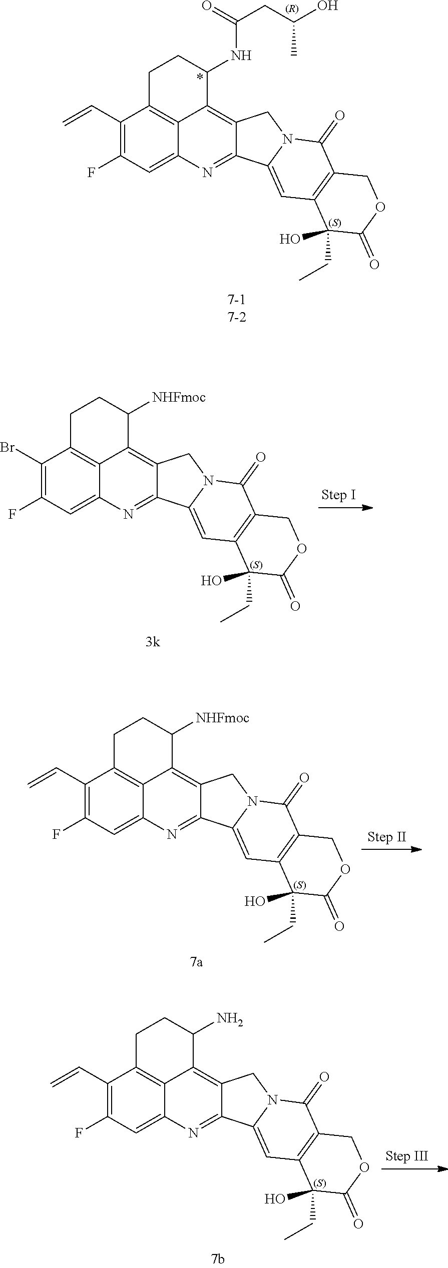

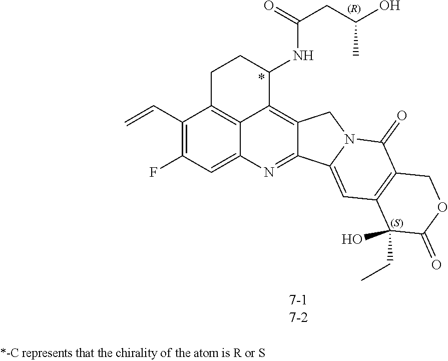

### Step I (9H-fluoren-9-yl)methyl((9S)-9-ethyl-5-fluoro-9-hydroxy-10,13-dioxo-4-ethenyl-2,3,9,10,13,15-hexahydro-1H,12H-benzo[de]pyrano[3′,4′,6,7]indolizino[1,2-b]quinolin-1-yl)carbamate 7a

The compound 3k (140 mg, 0.20 mmol) and potassium vinyltrifluoroborate (54 mg, 0.40 mmol) were dissolved in dioxane (16 mL) and water (4 mL), and potassium phosphate (128 mg, 0.60 mmol) and methanesulfonato(2-dicyclohexylphosphino-2′,6′-diisopropoxy-1,1′-biphenyl)(2-amino-1,1′-biphenyl-2-yl)palladium (II) (34 mg, 0.04 mmol) were added. After nitrogen replacement, the reaction mixture was stirred at 100° C. for 5 h. After the reaction was complete, water (20 mL) was added into the reaction mixture. The mixture was extracted with dichloromethane (15 mL/3). The organic phases were combined, washed with saturated brine, dried, and concentrated. The resulting residue was purified by silica gel column chromatography with the eluent system B, to give the title compound 7a (80 mg, yield: 60%).

MS m/z (ESI): 670.2 (M+1)⁺.

### Step II (S)-1-amino-9-ethyl-5-fluoro-9-hydroxy-4-ethenyl-1,2,3,9,12,15-hexahydro-10H,13H-benzo[de]pyrano[3′,4′,6,7]indolizino[1,2-b]quinoline-10,13-dione

The compound 7a (80 mg, 0.12 mmol) was dissolved in N,N-dimethylformamide (10 mL), diethylamine (1 mL) was added, and the reaction mixture was stirred at room temperature for 1 h. After the reaction was complete, the mixture was vacuum concentrated, slurried with ethyl acetate, and filtered to give the title compound 7b (30 mg, yield: 51%/).

MS m/z (ESI): 448.2 (M+1)⁺.

### Step III

### (R)—N-((1S,9S)-9-ethyl-5-fluoro-9-hydroxy-10,13-dioxo-4-ethenyl-2,3,9,10,13,15-hexahydro-1H,12H-benzo[de]pyrano[3′,4′,6,7]indolizino[1,2-b]quinolin-1-yl)-3-hydroxybutanamide

### (R)—N-((1R,9S)-9-ethyl-5-fluoro-9-hydroxy-10,13-dioxo-4-ethenyl-2,3,9,10,13,15-hexahydro-1H,12H-benzo[de]pyrano[3′,4′,6,7]indolizino[1,2-b]quinolin-1-yl)-3-hydroxybutanamide

The compound 7b (30 mg, 0.07 mmol) and (R)-3-hydroxybutyric acid (8 mg, 0.08 mmol) were dissolved in N,N-dimethylformamide (5 mL), and N,N-diisopropylethylamine (18.2 mg, 0.14 mmol) and 2-(7-azabenzotriazole)-N,N,N′,N′-tramethyluronium hexafluorophosphate (39.9 mg, 0.11 mmol) were added. The reaction mixture was stirred at 25° C. for 5 h. After the reaction was complete, the reaction mixture was concentrated, and purified by preparative high performance liquid chromatography (Waters MS-triggered Prep-LC with SQD2 detector, chromatographic column: Xbridge C18 150*19 mm, 5 μm; mobile phase 1: water (containing 0.1% formic acid); mobile phase 2: acetonitrile; 15-min gradient, gradient proportion: acetonitrile phase 43%-53%, flow rate: 20 mL/min) to give the title compound 7-1 (1.76 mg, yield: 10%) and the title compound 7-2 (2.31 mg, yield: 10%).

### Monoconfigurational Compound 7-1 (Short Retention Time):

MS m/z (ESI): 534.2 (M+1)⁺.

¹H NMR (400 MHz, DMSO-d₆) δ 8.51 (d, 1H), 8.48 (s, 1H), 7.84 (d, 1H), 7.32 (s, 1H), 6.97 (dd, 1H), 6.58 (s, 1H), 5.91-5.77 (m, 2H), 5.61-5.50 (m, 1H), 5.43 (s, 2H), 5.22 (q, 2H), 4.72 (s, 1H), 4.04 (dd, 1H), 3.27 (d, 1H), 2.30-2.20 (m, 2H), 2.11 (t, 2H), 1.90-1.80 (m, 2H), 1.09 (d, 3H), 0.87 (t, 3H).

### Monoconfigurational Compound 7-2 (Long Retention Time):

MS m/z (ESI): 534.2 (M+1)⁺.

¹H NMR (400 MHz, DMSO-d₆) δ 8.55 (d, 1H), 8.47 (s, 1H), 7.85 (d, 1H), 7.32 (s, 1H), 6.97 (dd, 1H), 6.62 (s, 1H), 5.82 (dd, 2H), 5.57 (dt, 1H), 5.43 (d, 2H), 5.24 (s, 2H), 4.72 (s, 1H), 4.03 (dt, 1H), 3.27 (d, 1H), 2.23 (ddd, 2H), 2.08 (dd, 2H), 1.87 (tt, 2H), 1.08 (d, 3H), 0.87 (s, 3H).

### Example 8

### (R)—N-((1S,9S)-9-ethyl-5-fluoro-9-hydroxy-10,13-dioxo-4-(2,2,2-trifluoroethoxy)-2,3,9,10,13,15-hexahydro-1H,12H-benzo[de]pyrano[3′,4′,6,7]indolizino[1,2-b]quinolin-1-yl)-3-hydroxybutanamide

### (R)—N-((1R,9S)-9-ethyl-5-fluoro-9-hydroxy-10,13-dioxo-4-(2,2,2-trifluoroethoxy)-2,3,9,10,13,15-hexahydro-1H,12H-benzo[de]pyrano[3′,4′,6,7]indolizino[1,2-b]quinolin-1-yl)-3-hydroxybutanamide

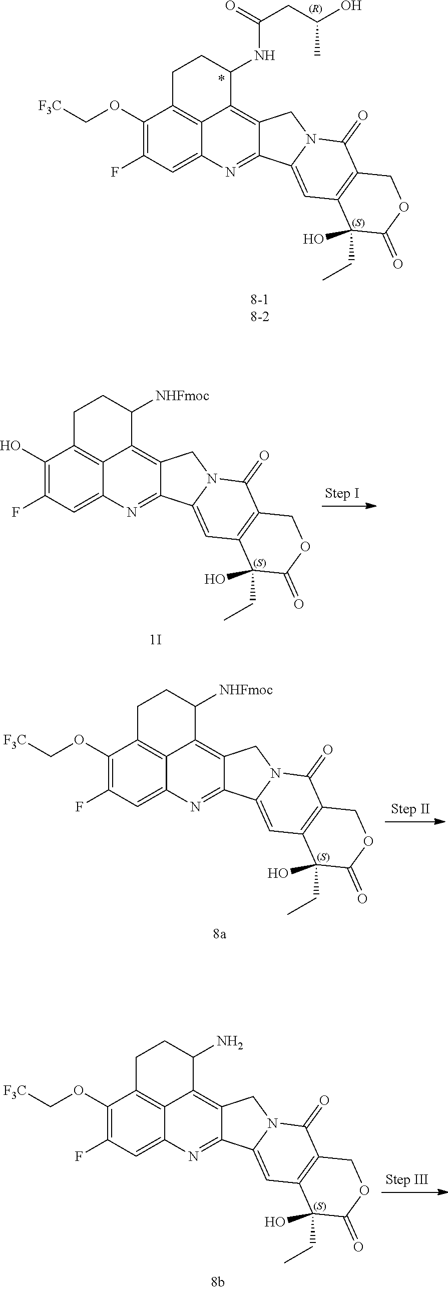

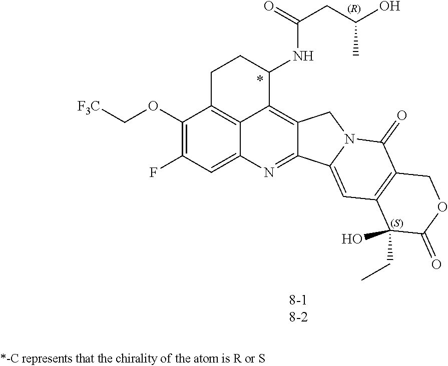

### Step I (9H-fluoren-9-yl)methyl((9S)-9-ethyl-5-fluoro-9-hydroxy-10,13-dioxo-4-(2,2,2-trifluoroethoxy)-2,3,9,10,13,15-hexahydro-1H,12H-benzo[de]pyrano[3′,4′,6,7]indolizino[1,2-b]quinolin-1-yl)carbamate 8a

The compound 11 (200 mg, 0.30 mmol) was dissolved in N,N-dimethylformamide (3 mL), and 2,2,2-trifluoroethyl trifluoromethanesulfonate (141 mg, 0.60 mmol) and cesium carbonate (296 mg, 0.90 mmol) were slowly added. The reaction mixture was stirred at room temperature for 10 min. After the reaction was complete, the mixture was diluted with water (10 mL), and extracted with ethyl acetate (10 mL×3). The organic phases were combined and concentrated. The resulting residue was purified by silica gel column chromatography with the eluent system B, to give the title compound 8a (180 mg, yield: 75%).

MS m/z (ESI): 742.2 (M+H)⁺.

### Step II (9S)-1-amino-9-ethyl-5-fluoro-9-hydroxy-4-(2,2,2-trifluoroethoxy)-1,2,3,9,12,15-hexahydro-10H,13H-benzo[de]pyrano[3′,4′,6,7]indolizino[1,2-b]quinoline-10,13-dione 8b

The compound 8a (180 mg, 0.24 mmol) was dissolved in N,N-dimethylformamide (3 mL), to which diethylamine (35 mg, 0.48 mmol) was added. The reaction mixture was kept for reaction at room temperature for 1 h. After the reaction was complete, the reaction mixture was concentrated under reduced pressure to give the crude product 8b (100 mg), which was directly used in the next reaction step without purification.

MS m/z (ESI): 520.1 (M+H)⁺.

### Step III

### (R)—N-((1S,9S)-9-ethyl-5-fluoro-9-hydroxy-10,13-dioxo-4-(2,2,2-trifluoroethoxy)-2,3,9,10,13,15-hexahydro-1H,12H-benzo[de]pyrano[3′,4′,6,7]indolizino[1,2-b]quinolin-1-yl)-3-hydroxybutanamide

### (R)—N-((1R,9S)-9-ethyl-5-fluoro-9-hydroxy-10,13-dioxo-4-(2,2,2-trifluoroethoxy)-2,3,9,10,13,15-hexahydro-1H,12H-benzo[de]pyrano[3′,4′,6,7]indolizino[1,2-b]quinolin-1-yl)-3-hydroxybutanamide

The compound 8b (50 mg, 0.09 mmol) was dissolved in N,N-dimethylformamide (2 mL), and (R)-3-hydroxybutyric acid (15 mg, 0.14 mmol), 2-(7-azabenzotriazole)-N,N,N′,N′-tramethyluronium hexafluorophosphate (73 mg, 0.19 mmol), and N,N-diisopropylethylamine (37 mg, 0.28 mmol) were added. The mixture was stirred for reaction at room temperature for 1 h. After the reaction was complete, the mixture was concentrated under reduced pressure to give the crude product, which was purified by preparative high performance liquid chromatography (GILSON Prep LC with UV detector, chromatographic column: Xtimate C18 150*19 mm, 10 m; mobile phase 1: water (containing 0.1% FA); mobile phase 2: acetonitrile; 15-min gradient, gradient proportion: acetonitrile phase 38%-48%, flow rate: 50 mL/min) to give the title compound 8-1 (2.5 mg, yield: 4.6%) and the title compound 8-2 (3.5 mg, yield: 6.4%).

### Monoconfigurational Compound 8-1 (Short Retention Time):

MS m/z (ESI): 606.2 (M+1)⁺.

¹H NMR (400 MHz, DMSO-d₆) δ 8.43 (d, 1H), 7.98 (d, 1H), 7.31 (s, 1H), 6.52 (s, 1H), 5.59-5.53 (m, 1H), 5.42 (s, 2H), 5.23 (s, 2H), 4.93-4.85 (m, 2H), 4.66 (d, 1H), 4.08-4.03 (m, 1H), 3.21 (d, 2H), 2.34-2.27 (m, 1H), 2.22 (d, 1H), 2.10 (dd, 2H), 1.89-1.81 (m, 2H), 1.10 (d, 3H), 0.90-0.84 (m, 3H).

### Monoconfigurational Compound 8-2 (Long Retention Time):

MS m/z (ESI): 606.2 (M+1)⁺.

¹H NMR (400 MHz, DMSO-d₆) δ 8.45 (d, 1H), 7.98 (d, 1H), 7.32 (s, 1H), 6.52 (s, 1H), 5.63-5.55 (m, 1H), 5.43 (s, 2H), 5.33-5.18 (m, 2H), 4.88 (dd, 2H), 4.65 (d, 1H), 4.09-4.00 (m, 1H), 3.24-3.12 (m, 2H), 2.28 (dd, 1H), 2.19 (dd, 1H), 2.09 (dd, 2H), 1.93-1.78 (m, 2H), 1.09 (d, 3H), 0.94-0.78 (m, 3H).

### Example 9

### (S)-9-ethyl-4,9-dihydroxy-1,2,3,9,12,15-hexahydro-10H,13H-benzopyrano[3′,4′,6,7]indolizino[1,2-b]quinoline-10,13-dione 9

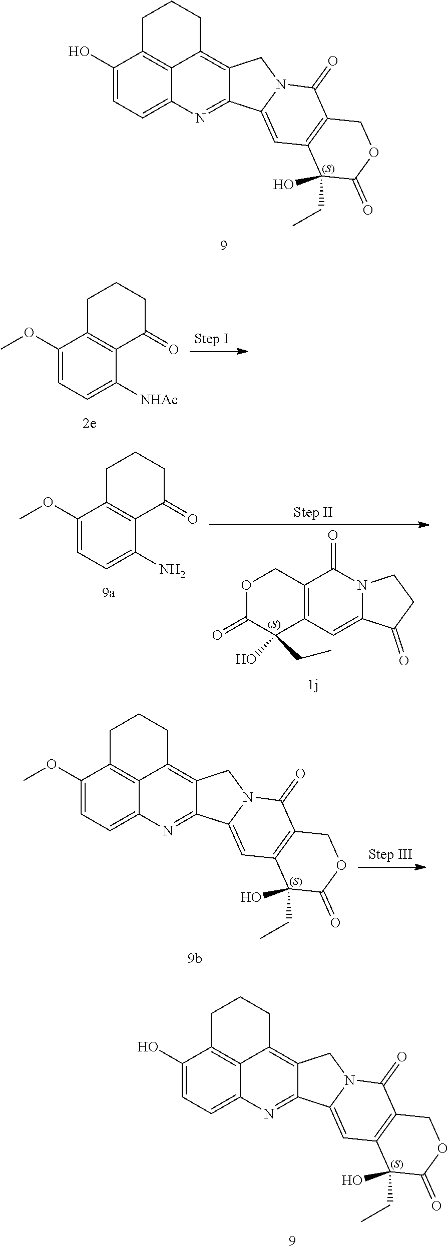

### Step I 8-amino-5-methoxy-3,4-dihydronaphthalen-1(2H)-one 9a

The compound 9e (500 mg, 2.14 mmol) was dissolved in a mixed solvent of methanol and concentrated hydrochloric acid (36 mL, V/V=5:1). The reaction mixture was kept for reaction at 60° C. for 2 h. After the reaction was complete, the reaction mixture was directly concentrated, to give the title compound 9a (400 mg, yield: 88%).

MS m/z (ESI): 192.1 (M+1)⁺.

### Step II (S)-9-ethyl-9-hydroxy-4-methoxy-1,2,3,9,12,15-hexahydro-10H,13H-benzopyrano[3′,4′,6,7]indolizino[1,2-b]quinoline-10,13-dione 9b

The compound 9a (400 mg, 2.09 mmol) was dissolved in toluene (15 mL), and the compound 1j (661 mg, 2.51 mmol) was added. The reaction mixture was stirred at 110° C. for 16 h. After the reaction was complete, water (20 mL) was added into the reaction mixture. The mixture was extracted with dichloromethane (15 mL×3). The organic phases were combined, washed with saturated brine, dried, and concentrated. The purification was performed by silica gel column chromatography with the eluent system B to give the title compound 9b (698 mg, yield: 80%).

MS m/z (ESI): 419.1 (M+1)⁺.

### Step III (S)-9-ethyl-4,9-dihydroxy-1,2,3,9,12,15-hexahydro-10H,13H-benzopyrano[3′,4′,6,7]indolizino[1,2-h]quinoline-10,13-dione 9

The compound 9b (100 mg, 0.24 mmol) was dissolved in hydrobromic acid (30 mL). The reaction mixture was kept for reaction at 100° C. for 2 h. After the reaction was complete, the reaction mixture was directly concentrated, and then purified by preparative high performance liquid chromatography (Waters MS triggered Prep-LC with Acquity QDA detector, chromatographic column: Welch C18 250×21.2 mm, 10 μm; mobile phase 1: water (containing 0.1% ammonia water); mobile phase 2: acetonitrile; 15-min gradient, gradient proportion: acetonitrile phase 10%-30%, flow rate: 25 mL/min) to give the title compound 9 (19.4 mg, yield: 20%).

MS m/z (ESI): 405.1 (M+1)⁺.

¹H NMR (400 MHz, CD₃OD) δ 7.79-7.71 (m, 2H), 7.29 (d, 1H), 5.01 (d, 2H), 4.95 (d, 2H), 3.01-2.86 (m, 4H), 2.43-2.35 (m, 1H), 2.28-2.19 (m, 1H), 2.06-2.00 (m, 2H), 1.10-1.00 (m, 3H).

### Example 10

### (1S,3R)—N-((1S,10S)-10-ethyl-6-fluoro-10-hydroxy-5-methyl-11,14-dioxo-1,2,3,4,10,11,14,16-octahydro-13H-cyclohepta[de]pyrano[3′,4′:6,7]indolizino[1,2-b]quinolin-1-yl)-3-hydroxycyclobutane-1-carboxamide

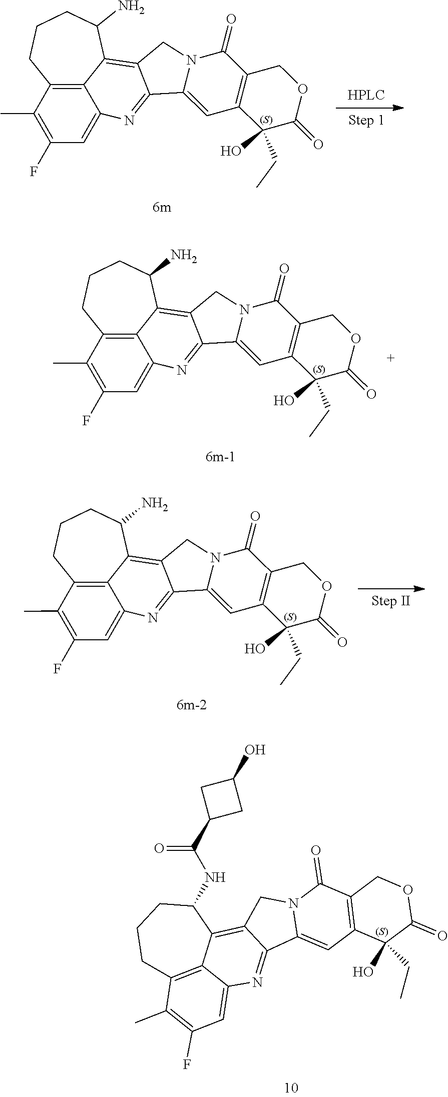

### Step I

### (1R,10S)-1-amino-10-ethyl-6-fluoro-10-hydroxy-5-methyl-2,3,4,10,13,16-hexahydro-14H-cyclohepta[3′,4′:6,7]indolizino[1,2-b]quinoline-11,14-(1H)-dione

### (1S,10S)-1-amino-10-ethyl-6-fluoro-10-hydroxy-5-methyl-2,3,4,10,13,16-hexahydro-4H-cyclohepta[3′,4′:6,7]indolizine[1,2-b]quinoline-11,14-(1H)-dione

Compound 6m (700 mg) was purified by preparative high performance liquid chromatography (GILSON Prep LC with UV detector, chromatographic column: Xbridge 5 μm C18 150×30 mm; mobile phase 1: water (0.1% TFA); mobile phase 2: acetonitrile; 15-min gradient, gradient proportion: acetonitrile phase 22%-32%, flow rate: 50 mL/min), to give compounds 6m-1 (290 mg, short retention time) and 6m-2 (300 mg, long retention time).

MS m/z (ESI): 450.2 (M+H)⁺

### Step II

### (1S,3R)—N-((1S,10S)-10-ethyl-6-fluoro-10-hydroxy-5-methyl-11,14-dioxo-1,2,3,4,10,11,14,16-octahydro-13H-cyclohepta[de]pyrano[3′,4′:6,7]indolizino[1,2-b]quinolin-1-yl)-3-hydroxycyclobutane-1-carboxamide 10

The compound 6m-2 (30 mg, 0.06 mmol) was dissolved in N,N-dimethylformamide (3 mL), and (1S,3S)-3-hydroxycyclobutane-1-carboxylic acid (8.5 mg, 0.07 mmol), 2-(7-azabenzotriazole)-N,N,N′,N′-tramethyluronium hexafluorophosphate (38 mg, 0.10 mmol), and N,N-diisopropylethylamine (17.2 mg, 0.13 mmol) were added. The reaction mixture was stirred at room temperature for 1 h. After the reaction was complete, the reaction mixture was diluted with water (10 mL), and extracted with ethyl acetate (10 mL×3). The organic phases were combined, washed with saturated brine, dried, and concentrated to give the crude product, which was purified by preparative high performance liquid chromatography (GILSON Prep LC with UV detector, chromatographic column: Xbridge 5 μm C18 150×30 mm; mobile phase 1: water (containing 0.1% TFA); mobile phase 2: acetonitrile; 15-min gradient, gradient proportion: acetonitrile phase 30%-95%, flow rate: 50 mL/min) to give the compound 10 (2 mg, yield: 5%).

MS m/z (ESI): 548.2 (M+H)⁺.

¹H NMR (400 MHz, CD₃OD) δ 8.71 (d, 1H), 7.57-7.48 (m, 2H), 5.65 (s, 1H), 5.55 (d, 1H), 5.35 (dd, 2H), 5.10 (d, 1H), 4.13 (dd, 1H), 2.73 (dd, 1H), 2.61-2.33 (m, 8H), 2.20-2.08 (m, 4H), 1.96 (dd, 4H), 1.02-0.97 (m, 3H).

### Example 11

### (R)-2-cyclopropyl-N-((1S,10S)-10-ethyl-6-fluoro-10-hydroxy-5-methyl-11,14-dioxo-1,2,3,4,10,11,14,16-octahydro-13H-cyclohepta[de]pyrano[3′,4′:6,7]indolizino[1,2-b]quinolin-1-yl)-2-hydroxyacetamide

### (S)-2-cyclopropyl-N-((1S,10S)-10-ethyl-6-fluoro-10-hydroxy-5-methyl-11,14-dioxo-1,2,3,4,10,11,14,16-octahydro-13H-cyclohepta[de]pyrano[3′,4′:6,7]indolizino[1,2-b]quinolin-1-yl)-2-hydroxyacetamide

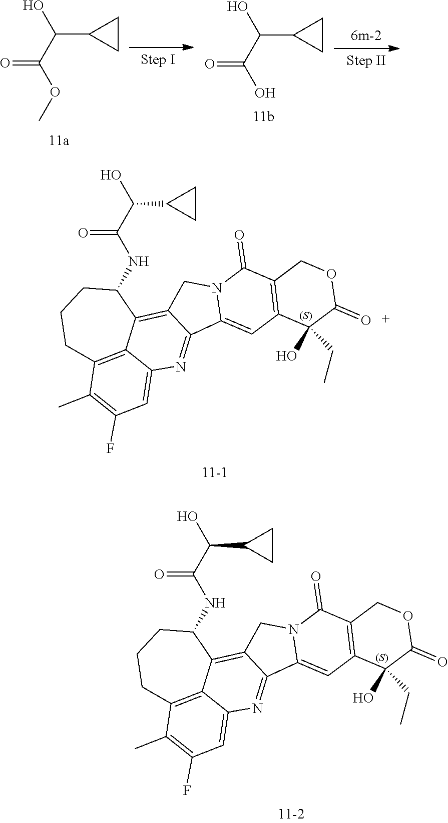

### Step I 2-cyclopropyl-2-hydroxyacetic acid 11b

Methyl 2-cyclopropyl-2-hydroxyacetate 11a (50 mg, 0.38 mmol) was dissolved in tetrahydrofuran (6 mL), and water (0.5 mL) and lithium hydroxide (27 mg, 1.15 mmol) were added. The mixture was stirred for reaction at room temperature for 1 h. After the reaction was complete, the mixture was spin-dried to remove the solvent, thus giving the crude product 2-cyclopropyl-2-hydroxyacetic acid 11b (50 mg, white solid), which was directly used in the next reaction step without purification.

MS m/z (ESI): 117.1 (M+H)⁺.

### Step II

### (R)-2-cyclopropyl-N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-2-hydroxyacetamide

### (S)-2-cyclopropyl-N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-2-hydroxyacetamide

The compound 6m-2 (50 mg, 0.11 mmol) was dissolved in N,N-dimethylformamide (2 mL), and 2-cyclopropyl-2-hydroxyacetic acid 11b (44 mg, 0.38 mmol), N,N-diisopropylethylamine (29 mg, 0.22 mmol), and 2-(7-azabenzotriazole)-N,N,N′,N′-tetramethyluronium hexafluorophosphate (63 mg, 0.17 mmol) were added. The mixture was stirred for reaction at room temperature for 15 min. After the reaction was complete, the purification was performed by preparative high performance liquid chromatography (Waters MS-triggered Prep-LC with SQD2 detector, chromatographic column: Xbridge 5 μm C18 150×19 mm; mobile phase 1: water (containing 0.1% FA); mobile phase 2: acetonitrile; 10-min gradient, gradient proportion: acetonitrile phase 40%-50%, flow rate: 20 mL/min) to give compound 11-1 (2.3 mg, yield: 4%) and compound 11-2 (2.5 mg, yield: 4%).

Monoconfigurational Compound 11-1 (Compound with Short Retention Time)

MS m/z (ESI): 548.2 (M+H)⁺.

¹H NMR (400 MHz, DMSO-d₆) δ 8.49 (d, 1H), 7.74 (d, 1H), 7.28 (s, 1H), 6.50 (s, 1H), 5.56 (s, 1H), 5.49-5.40 (m, 4H), 5.29 (d, 1H), 3.61 (t, 1H), 3.22 (s, 2H), 2.42 (s, 3H), 2.12-1.63 (m, 6H), 1.11 (d, 1H), 0.87 (t, 3H), 0.47-0.27 (m, 4H).

Monoconfigurational Compound 11-2 (Compound with Long Retention Time)

MS m/z (ESI): 548.2 (M+H)⁺.

¹H NMR (400 MHz, DMSO-d₆) δ 8.43 (d, 1H), 7.74 (d, 1H), 7.28 (s, 1H), 6.51 (s, 1H), 5.54 (s, 1H), 5.42 (t, 4H), 5.31 (d, 1H), 3.68-3.53 (m, 1H), 3.22 (s, 2H), 2.42 (s, 3H), 2.12-1.66 (m, 6H), 1.10 (d, 1H), 0.87 (t, 3H), 0.48-0.28 (m, 4H).

### Example 12

### (1S,3R)—N-((1S,9S)-9-ethyl-5-fluoro-9-hydroxy-10,13-dioxo-4-ethenyl-2,3,9,10,13,15-hexahydro-1H,12H-benzo[d]pyrano[3′,4′:6,7]indolizino[1,2-b]quinolin-1-yl)-3-hydroxycyclobutane-1-carboxamide

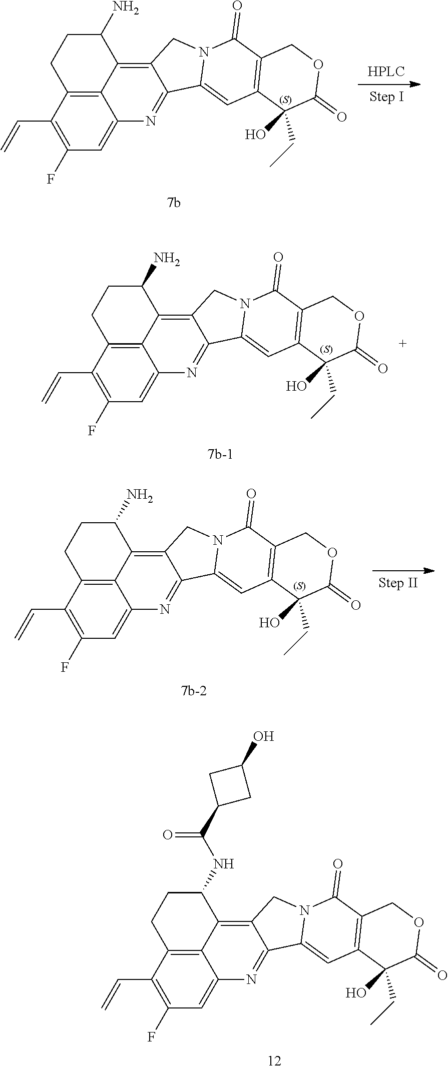

### Step I (1R,9S)-1-amino-9-ethyl-5-fluoro-9-hydroxy-4-ethenyl-1,2,3,9,12,15-hexahydro-10H,13H-benzo[de]pyrano[3′,4′:6,7]indolizino[1,2-b]quinoline-10,13-dione

### (1S,9S)-1-amino-9-ethyl-5-fluoro-9-hydroxy-4-ethenyl-1,2,3,9,12,15-hexahydro-10H,13H-benzo[de]pyrano[3′,4′:6,7]indolizino[1,2-b]quinoline-10,13-dione

The compound 7b (500 mg) was purified by preparative high performance liquid chromatography (GILSON Prep LC with UV detector, chromatographic column: Xbridge 5 μm C18 150×30 mm; mobile phase 1: water (0.1% TFA); mobile phase 2: acetonitrile; 15-min gradient, gradient proportion: acetonitrile phase 22%-32%, flow rate: 50 mL/min), to give compounds 7b-1 (180 mg, short retention time) and 7b-2 (195 mg, long retention time).

MS m/z (ESI): 448.2 (M+H)⁺.

### Step II (1S,3R)—N-((1S,9S)-9-ethyl-5-fluoro-9-hydroxy-10,13-dioxo-4-ethenyl-2,3,9,10,13,15-hexahydro-1H,12H-benzo[d]pyrano[3′,4′:6,7]indolizino[1,2-b]quinolin-1-yl)-3-hydroxycyclobutane-1-carboxamide 12

The compound 7b-2 (22 mg, 0.05 mmol) was dissolved in N,N-dimethylformamide (0.5 mL), and (1S,3S)-3-hydroxycyclobutane-1-carboxylic acid (7 mg, 0.06 mmol), 2-(7-azabenzotriazole)-N,N,N′N′-tramethyluronium hexafluorophosphate (30 mg, 0.08 mmol), and N,N-diisopropylethylamine (13 mg, 0.10 mmol) were added. The mixture was stirred for reaction at room temperature for 15 min. After the reaction was complete, the reaction mixture was purified by preparative high performance liquid chromatography (Waters MS-triggered Prep-LC with SQD2 detector, chromatographic column: Xbridge 5 μm C18 150×19 mm; mobile phase 1: water (containing 0.1% FA); mobile phase 2: acetonitrile; 10-min gradient, gradient proportion: acetonitrile phase 45%-100%, flow rate: 20 mL/min) to give the compound 12 (3 mg, yield: 11%).

MS m/z (ESI): 546.2 (M+H)⁺.

¹H NMR (400 MHz, CD₃OD) δ 8.50 (s, 1H), 7.71 (d, 1H), 7.64 (s, 1H), 6.95 (dd, 1H), 5.83 (dd, 2H), 5.67-5.61 (m, 11H), 5.57 (d, 1H), 5.39 (d, 1H), 5.24 (d, 2H), 4.16-4.05 (m, 1H), 3.45-3.35 (m, 2H), 2.73-2.42 (m, 4H), 2.29-2.17 (m, 4H), 2.01-1.91 (m, 2H), 1.00 (t, 3H).

### Example 13

### (R)—N-((1R,9S)-4-cyclopropyl-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)-3-hydroxybutanamide

### (R)—N-((1S,9S)-4-cyclopropyl-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)-3-hydroxybutanamide

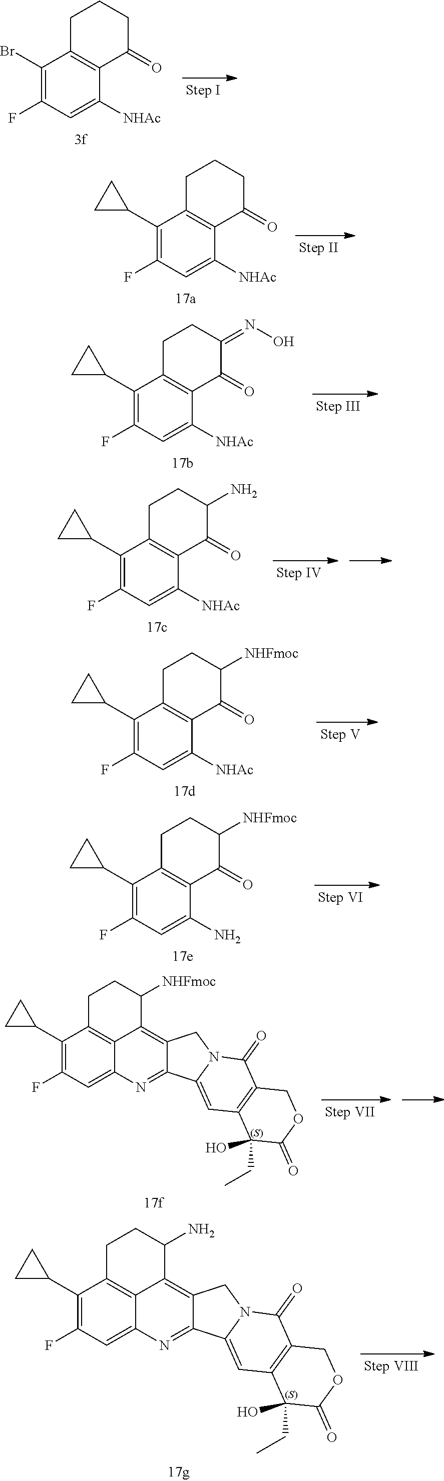

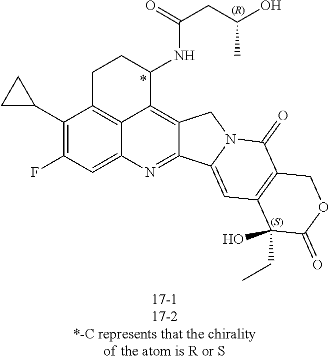

### Step I N-(4-cyclopropyl-3-fluoro-8-oxo-5,6,7,8-tetralin-1-yl)acetamide 17a

The compound 3 (1.5 g, 5.02 mmol), cyclopropylboronic acid (1.3 g, 15.06 mmol), [,1-bis(diphenylphosphino)ferrocene]dichloropalladium (2.2 g, 3.01 mmol), and cesium carbonate (1.6 g, 15.06 mmol) were dissolved in dioxane (15 mL). The mixture was stirred for reaction in a microwave reactor at 110° C. for 2 h. After the reaction was complete, the reaction mixture was poured into water, extracted with ethyl acetate three times, and dried over anhydrous sodium sulfate. The organic phases were combined, concentrated, and purified by silica gel column chromatography with the eluent system B to give the title compound 17a (1.1 g, yield: 84%).

MS m/z (ESI): 262.1 (M+)⁺.

### Step II (Z)—N-(4-cyclopropyl-3-fluoro-7-(hydroxyimino)-8-oxo-5,6,7,8-tetralin-1-yl)acetamide 17b

Potassium tert-butoxide (0.94 g, 8.4 mmol) was dissolved in a mixed solvent of tetrahydrofuran and tert-butanol (50 mL, V/V=4:1), the compound 17a (1.1 g, 4.2 mmol) was dissolved in 10 mL of the tetrahydrofuran solution, and the mixture was slowly added to the reaction system at 0° C. After the mixture was stirred for reaction at 0° C. for 10 min, isoamyl nitrite (0.74 g, 6.3 mmol) was added, and the reaction was continued by stirring at 0° C. for 50 min. After the reaction was complete, the system was adjusted with dilute hydrochloric acid to a pH of 4-5, and extracted with ethyl acetate 3 times. The organic phase was washed with saturated brine, collected, dried over anhydrous sodium sulfate, and spin-dried to remove the solvent, thus giving the crude product 17b, which was directly used in the next reaction step without purification.

MS m/z (ESI): 291.0 (M+1)⁺.

### Step III N-(7-amino-4-cyclopropyl-3-fluoro-8-oxo-5,6,7,8-tetralin-1-yl)acetamide 17c

The compound 17b was dissolved in methanol (50 mL), and a palladium-carbon catalyst (500 mg) was added. The mixture was stirred for reaction in a hydrogen environment at room temperature for 2 h. After the reaction was complete, the mixture was filtered. The filtrate was collected to give the crude product 17c, which was directly used in the next reaction step without purification.

MS m/z (ESI): 277.1 (M+1)⁺.

### Step IV (9H-fluoren-9-yl)methyl(8-acetylamino-5-cyclopropyl-6-fluoro-1-oxo-1,2,3,4-tetralin-2-yl)carbamate 17d

The filtrate obtained from the previous step was adjusted with a saturated sodium carbonate solution to a PH of 8-9, and then Fmoc-Cl (1.19 g, 4.6 mmol) was added. The mixture was stirred for reaction at room temperature for 1 h. After the reaction was complete, the system was extracted with ethyl acetate. The organic phase was washed with saturated brine, collected, dried over anhydrous sodium sulfate, and spin-dried to remove the solvent, thus giving the crude product, which was purified through the silica gel column chromatography system A to give the compound 17d (1 g, yield: 48%).

MS m/z (ESI): 499.1 (M+1)⁺.

### Step V (9H-fluoren-9-yl)methyl(8-amino-5-cyclopropyl-6-fluoro-1-oxo-1,2,3,4-tetralin-2-yl)carbamate 17e

The compound 17e (1 g, 2.01 mmol) was dissolved in dioxane (20 mL), and concentrated hydrochloric acid (12 mol/L, 5 mL) was added. The mixture was stirred for reaction at 60° C. for 1 h. After the reaction was complete, the mixture was spin-dried to remove the solvent, thus giving the crude product, which was purified through the silica gel column chromatography system A to give compound 17f (700 mg, yield: 77%).

MS m/z (ESI): 457.1 (M+1).

### Step VI (9H-fluoren-9-yl)methyl((9S)-4-cyclopropyl-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)carbamate 17g

The compound 17f (700 mg, 1.54 mmol) was dissolved in toluene (10 mL), and (S)-4-ethyl-4-hydroxy-7,8-dihydro-1H-pyrano[3,4-f]indolizine-3,6,10(4H)-trione (484 mg, 1.84 mmol), and p-toluenesulfonic acid monohydrate (292 mg, 1.54 mmol) were added. The reaction system was stirred for reaction at 140° C. for 4 h. After the reaction was complete, the mixture was spin-dried to remove the solvent, thus giving the crude product, which was purified through a silica gel column chromatography system Achu to give the title compound 17g (700 mg, yield: 66%).

MS m/z (ESI): 684.2 (M+1)⁺.

Step VII (9S)-1-amino-4-cyclopropyl-9-ethyl-5-fluoro-9-hydroxy-1,2,3,9,12,15-hexahydro-10H,13H-benzo[d]pyrano[3′,4′:6,7]indolizino[1,2-b]quinoline-10,13-dione 17h

The compound 17g (100 mg, 0.15 mmol) was dissolved in N,N-dimethylformamide (5 mL), and diethylamine (0.5 mL) was added. The mixture was stirred for reaction at room temperature for 30 min. After the reaction was complete, the mixture was vacuum concentrated to give the crude compound 17h (60 mg), which was directly used in the next reaction step without purification.

MS m/z (ESI): 462.1 (M+1)⁺.

### Step VIII (R)—N-((1R,9S)-4-cyclopropyl-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)-3-hydroxybutanamide

### (R)—N-((1S,9S)-4-cyclopropyl-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)-3-hydroxybutanamide

The compound 17h (60 mg, 0.13 mmol) was dissolved in DMF (5 mL), and then (R)-3-hydroxybutyric acid (19 mg, 0.18 mmol), 2-(7-azabenzotriazole)-N,N,N′N′-tramethyluronium hexafluorophosphate (87 mg, 0.23 mmol), and N,N-dimethylacetamide (59 mg, 0.45 mmol) were added. The mixture was stirred for reaction at room temperature for 30 min. After the reaction was complete, the reaction mixture was purified by preparative high performance liquid chromatography (Waters MS-triggered Prep-LC with SQD2 detector, chromatographic column: Xbridge 5 μm C18 150×19 mm; mobile phase 1: water (containing 0.1% FA); mobile phase 2: acetonitrile; 15-min gradient, gradient proportion: acetonitrile phase 38%-48%, flow rate: 20 mL/min) to give compounds 17-1 (6 mg, yield: 11%) and 17-2 (4 mg, yield: 8%).

### Monoconfigurational Compound 17-1 (Short Retention Time):

MS m/z (ESI): 548.2 (M+1)⁺.

¹H NMR (400 MHz, DMSO-d₆) δ 8.44 (d, 1H), 7.75 (d, 1H), 7.47 (d, 1H), 7.30 (s, 1H), 7.11 (d, 1H), 5.60-5.52 (m, 1H), 5.43 (s, 2H), 5.29-5.13 (m, 2H), 4.09-4.00 (m, 1H), 2.48 (s, 1H), 2.29 (s, 2H), 2.24-2.18 (m, 1H), 2.14 (d, 2H), 1.99-1.92 (m, 1H), 1.91-1.79 (m, 2H), 1.15-1.10 (m, 2H), 1.09 (d, 3H), 0.87 (s, 3H), 0.76 (s, 2H).

### Monoconfigurational Compound 17-2 (Long Retention Time):

MS m/z (ESI): 548.2 (M+1)⁺.

¹H NMR (400 MHz, DMSO-d₆) δ 8.46 (d, 1H), 7.76 (d, 1H), 7.47 (d, 1H), 7.30 (s, 1H), 7.13-7.06 (m, 1H), 5.59 (dd, 1H), 5.43 (d, 2H), 5.24 (d, 2H), 4.05 (dd, 1H), 2.29 (t, 1H), 2.25 (d, 1H), 2.19 (d, 1H), 2.18-2.06 (m, 3H), 1.95 (d, 1H), 1.85 (dd, 2H), 1.16-1.10 (m, 2H), 1.08 (d, 3H), 0.87 (s, 4H), 0.76 (d, 2H).

### Example 14

### (1S,3S)—N-((1R,9S)-4-cyclopropyl-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)-3-hydroxycyclobutane-1-carboxamide

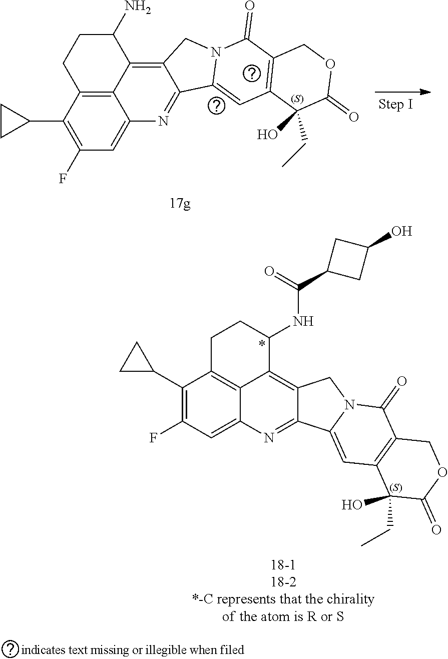

### Step I

### (1S,3S)—N-((1R,9S)-4-cyclopropyl-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)-3-hydroxycyclobutane-1-carboxamide

### (1S,3R)—N-((1S,9S)-4-cyclopropyl-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)-3-hydroxycyclobutane-1-carboxamide

The compound 17g (69 mg, 0.15 mmol) was dissolved in N,N-dimethylformamide (5 mL), and (1S,3S)-3-hydroxycyclobutane-1-carboxylic acid (21 mg, 0.18 mmol), 2-(7-azabenzotriazole)-N,N,N′,N′-tramethyluronium hexafluorophosphate (87 mg, 0.23 mmol), and N,N-dimethylacetamide (59 mg, 0.45 mmol) were added. The mixture was stirred for reaction at room temperature for 30 min. After the reaction was complete, the reaction mixture was purified by preparative high performance liquid chromatography (Waters MS-triggered Prep-LC with QDA detector, chromatographic column: WELCH Xtimate C18 21.2×250 mm 10 μm; mobile phase 1: water (containing 0.1% FA); mobile phase 2: acetonitrile; 18-min gradient, gradient proportion: acetonitrile phase 35%-65%, flow rate: 20 mL/min) to give compounds 18-1 (4 mg, yield: 6%) and 18-2 (2.4 mg, yield: 4%:) Monoconfigurational compound 18-1 (short retention time):

MS m/z (ESI): 560.2 (M+1)⁺.

¹H NMR (400 MHz, DMSO-d₆) δ 8.44 (d, 1H), 7.71 (d, 1H), 7.29 (d, 1H), 6.54 (d, 1H), 5.56 (s, 1H), 5.42 (s, 2H), 5.17-5.09 (m, 3H), 3.94 (d, 1H), 2.44-2.30 (m, 4H), 2.08-2.03 (m, 4H), 1.94-1.84 (m, 4H), 1.11 (d, 2H), 0.87 (t, 3H), 0.76 (s, 2H).

### Monoconfigurational Compound 18-2 (Long Retention Time):

MS m/z (ESI): 560.2 (M+1)⁺.

¹H NMR (400 MHz, DMSO-d₆) δ 8.45 (d, 1H), 7.74 (d, 1H), 7.30 (s, 1H), 6.53 (s, 1H), 5.57 (s, 1H), 5.43 (s, 2H), 5.14 (dd, 3H), 3.97-3.91 (m, 1H), 2.40-2.27 (m, 4H), 2.15-2.05 (m, 4H), 1.96-1.84 (m, 4H), 1.11 (d, 2H), 0.87 (t, 3H), 0.75 (d, 2H).

### Example 15

### (R)—N-((1S,10S)-5-cyclopropyl-10-ethyl-6-fluoro-10-hydroxy-11,14-dioxo-1,2,3,4,10,14,16-octahydro-13H-cyclohepta[de]pyrano[3′,4′:6,7]indolizino[1,2-b]quinolin-1-yl)-3-hydroxybutanamide

### (R)—N-((1R,10S)-5-cyclopropyl-10-ethyl-6-fluoro-10-hydroxy-11,14-dioxo-1,2,3,4,10,14,16-octahydro-13H-cyclohepta[de]pyrano[3′,4′:6,7]indolizino[1,2-b]quinolin-1-yl)-3-hydroxybutanamide

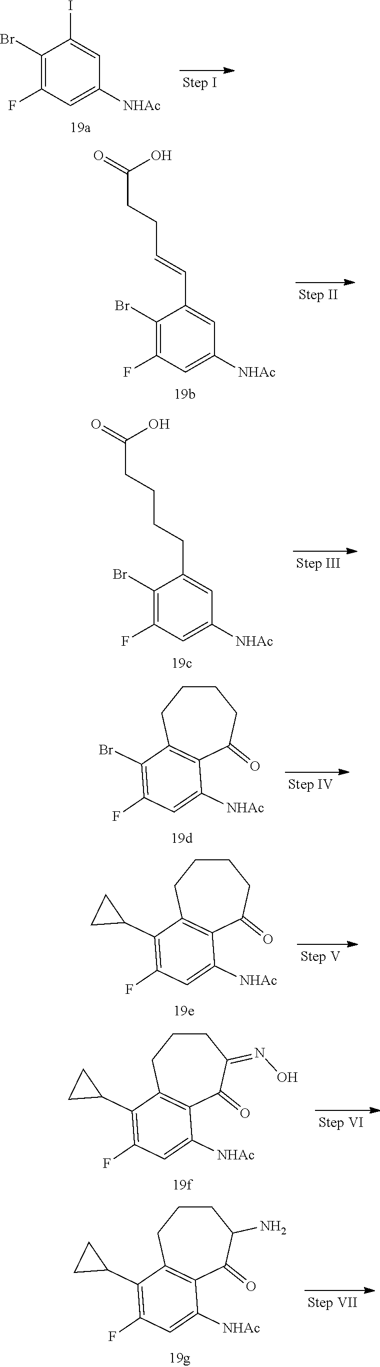

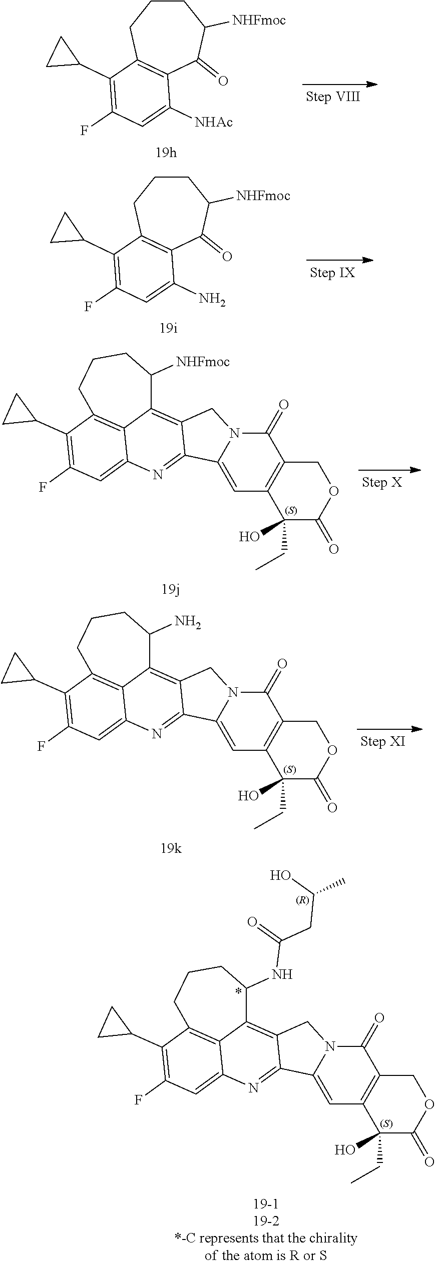

### Step I 5-(5-acetylamino-2-bromo-3-fluorophenyl)pent-4-enoic acid 19b

The compound 19a (7.0 g, 19.5 mmol) was dissolved in dioxane (60 mL) and water (15 mL), to which pent-4-enoic acid (2.2 g, 22.0 mol), bis(triphenylphosphine)palladium chloride (700 mg, 1.0 mmol), and sodium carbonate (6.2 g, 58.4 mmol) were added. The reaction mixture was stirred under the protection of nitrogen at 90° C. for 16 h. After the reaction was complete, the reaction mixture was filtered through diatomite, and washed with ethyl acetate. The resulting filtrate was directly concentrated. The crude product was diluted with water (200 mL), and extracted with diethyl ether 3 times (100 mL×3). The aqueous phase was adjusted with dilute hydrochloric acid to pH=2, and extracted with ethyl acetate (100 mL×3). The organic phases were combined and concentrated to give the crude product 19b (6.0 g).

MS m/z (ESI): 330.1 (M+1)⁺.

### Step II 5-(5-acetylamino-2-bromo-3-fluorophenyl)pentanoic acid 19c

The compound 19b (6.0 g, 18.2 mmol) was dissolved in anhydrous methanol (100 mL), to which platinum/carbon (5%, 600 mg) was added. The reaction mixture was subjected to hydrogen replacement three times, and stirred for reaction at room temperature for 16 h. After the reaction was complete, the reaction mixture was filtered through diatomite, and washed with ethyl acetate. The resulting filtrate was directly concentrated to give the crude compound 19c (5.0 g).

MS m/z (ESI): 332.0 (M+1)⁺.

### Step III N-(4-bromo-3-fluoro-9-oxo-6,7,8,9-tetrahydro-5H-benzo[7]cycloalken-1-yl)acetamide 19d

The compound 19c (2.5 g, 3.3 mmol) was dissolved in polyphosphoric acid (25 mL). The reaction mixture was stirred for reaction at 140° C. for 2 h. After the reaction was complete, water (100 mL) was added to the reaction mixture, and the mixture was stirred for 4 h. The aqueous phase was extracted with ethyl acetate (100 mL×3). The organic phases were combined and concentrated. The resulting crude product was purified through the silica gel column chromatography system B to give the compound 19d (920 mg, yield: 39%).

MS m/z (ESI): 314.0 (M+1)⁺.

A synthetic route same as that in Example 17 was used as the subsequent synthetic route, except that the compound 3M was replaced with the compound 19d (720 mg, 2.3 mmol). The final product was purified by preparative high performance liquid chromatography (Waters MS-triggered Prep-LC with QDA detector, chromatographic column: WELCH Xtimate C18 21.2×250 mm 10 μm; mobile phase 1: water (containing 0.1% FA); mobile phase 2: acetonitrile; 15-min gradient, gradient proportion: acetonitrile phase 40%-95%, flow rate: 30 mL/min) to give products 19-1 (6.5 mg, yield: 9%) and 19-2 (1 μmg, yield: 16%).

### Monoconfigurational Compound 19-1 (Short Retention Time):

MS m/z (ESI): 562.2 (M+1)⁺.

¹H NMR (400 MHz, CDCl₃) δ 7.63-7.50 (m, 3H), 7.07 (s, 1H), 5.73-5.69 (m, 2H), 5.43 (d, 1H), 5.36-5.15 (m, 2H), 4.24 (s, 1H), 3.81 (s, 1H), 3.64-3.50 (m, 1H), 3.47-3.32 (m, 1H), 2.52-2.41 (m, 2H), 2.36-2.12 (m, 3H), 1.88-1.83 (m, 4H), 1.25 (d, 3H), 1.19-1.15 (m, 2H), 1.02 (t, 3H), 0.81-0.77 (m, 2H).

### Monoconfigurational Compound 19-2 (Long Retention Time):

MS m/z (ESI): 562.2 (M+1)⁺.

¹H NMR (400 MHz, CDCl₃) δ 7.50-7.35 (m, 3H), 5.72-5.55 (m, 1H), 5.47-5.29 (m, 2H), 5.26-5.05 (m, 2H), 4.51-4.31 (m, 1H), 4.05-3.91 (m, 1H), 3.70-3.54 (m, 1H), 3.53-3.37 (m, 1H), 2.66-2.50 (m, 2H), 2.49-2.37 (m, 1H), 2.28-2.14 (m, 1H), 2.11-1.75 (m, 5H), 1.41-1.29 (m, 3H), 1.29-1.10 (m, 2H), 1.04-0.95 (m, 3H), 0.88-0.71 (m, 2H).

### Example 16

### (1S,3S)—N-((1R,10S)-5-cyclopropyl-10-ethyl-6-fluoro-10-hydroxy-11,14-dioxo-1,2,3,4,10,11,14,16-octahydro-13H-cyclohepta[de]pyrano[3′,4′:6,7]indolizino[1,2-b]quinolin-1-yl)-3-hydroxybutane-1-carboxamide

### (1S,3S)—N-((1S,10S)-5-cyclopropyl-10-ethyl-6-fluoro-10-hydroxy-11,14-dioxo-1,2,3,4,10,11,14,16-octahydro-13H-cyclohepta[de]pyrano[3′,4′:6,7]indolizino[1,2-b]quinolin-1-yl)-3-hydroxybutane-1-carboxamide

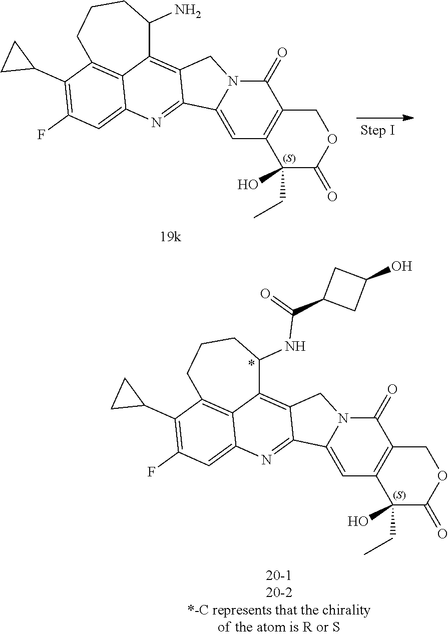

### Step I

### (1S,3S)—N-((1R,10S)-5-cyclopropyl-10-ethyl-6-fluoro-10-hydroxy-11,14-dioxo-1,2,3,4,10,11,14,16-octahydro-13H-cyclohepta[de]pyrano[3′,4′:6,7]indolizino[1,2-b]quinolin-1-yl)-3-hydroxybutane-1-carboxamide

### (1S,3S)—N-((1S,10S)-5-cyclopropyl-10-ethyl-6-fluoro-10-hydroxy-11,14-dioxo-1,2,3,4,10,11,14,16-octahydro-13H-cyclohepta[de]pyrano[3′,4′:6,7]indolizino[1,2-b]quinolin-1-yl)-3-hydroxybutane-1-carboxamide

The compound 19k (75 mg, 0.16 mmol) was dissolved in N,N-dimethylformamide (3 mL), and (1S,3S)-3-hydroxycyclobutane-1-carboxylic acid (22 mg, 0.19 mmol), 2-(7-azabenzotriazole)-N,N,N′N′-tramethyluronium hexafluorophosphate (91 mg, 0.24 mmol), and N,N-diisopropylethylamine (171 mg, 1.32 mmol) were added. The reaction mixture was stirred for reaction at room temperature for 1 h. After the reaction was complete, the reaction mixture was diluted with water (50 mL), and extracted with ethyl acetate (30 mL×3). The organic phases were combined, washed with saturated brine, dried, and concentrated. The crude product was purified by preparative high performance liquid chromatography (GILSON Prep LC with UV detector, chromatographic column: WELCH Xtimate C18 21.2×250 mm 10 μm; mobile phase 1: water (containing 0.1% NH3·H2O); mobile phase 2: acetonitrile; 15-min gradient, gradient proportion: acetonitrile phase 40%-100%, flow rate: 30 mL/min) to give compounds 20-1 (2.9 mg, 7%) and 20-2 (1.5 mg, 2%).

### Monoconfigurational Compound 20-1 (Short Retention Time):

MS m/z (ESI): 574.7 (M+1)⁺.

1H NMR (400 MHz, CDCl₃) δ 7.59 (d, 1H), 7.53 (s, 1H), 6.49 (s, 1H), 5.72-5.64 (m, 1H), 5.59 (d, 1H), 5.35 (d, 1H), 5.14 (d, 1H), 4.93 (d, 1H), 4.25-4.19 (m, 1H), 3.77 (s, 1H), 3.64-3.52 (m, 1H), 3.46-3.41 (m, 1H), 2.77-2.68 (m, 3H), 2.42-2.10 (m, 5H), 1.94-1.78 (m, 4H), 1.25-1.75 (m, 2H), 1.05 (t, 3H), 0.87-0.72 (m, 2H).

### Monoconfigurational Compound 20-2 (Long Retention Time):

MS m/z (ESI): 574.7 (M+1)⁺.

¹H NMR (400 MHz, CDCl₃) δ 7.53 (d, 1H), 7.46 (s, 1H), 6.91 (br, 1H), 5.59 (t, 1H), 5.30-5.19 (m, 2H), 5.11-5.04 (m, 2H), 4.30-4.21 (m, 1H), 3.76 (s, 1H), 3.68 (d, 1H), 3.48-3.34 (m, 1H), 2.88-2.75 (m, 3H), 2.37-2.21 (m, 5H), 1.93 (s, 3H), 1.80-1.72 (m, 2H), 1.23-1.13 (m, 2H), 0.96 (t, 3H), 0.87-0.75 (m, 2H).

### Example 17

### (R)-2-cyclopropyl-N-((1S,10S)-5-cyclopropyl-10-ethyl-6-fluoro-10-hydroxy-11,14-dioxo-1,2,3,4,10,11,14,16-octahydro-13H-cyclohepta[3′,4′:6,7]indolizino[1,2-b]quinolin-1-yl)-2-hydroxyacetamide

### (S)-2-cyclopropyl-N-((1S,10S)-5-cyclopropyl-10-ethyl-6-fluoro-10-hydroxy-11,14-dioxo-1,2,3,4,10,11,14,16-octahydro-13H-cyclohepta[3′,4′:6,7]indolizino[1,2-b]quinolin-1-yl)-2-hydroxyacetamide

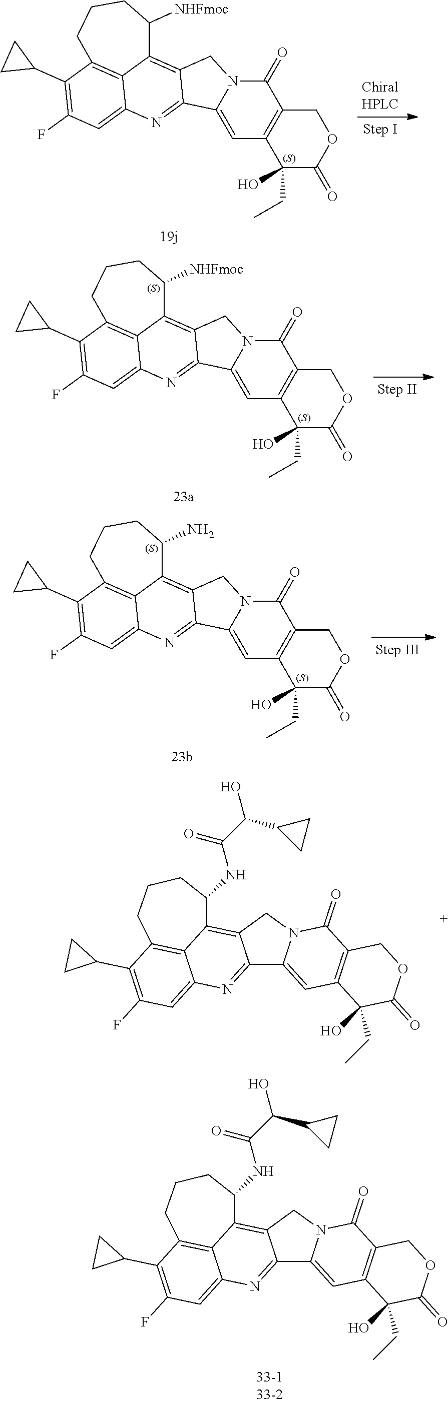

### Step I (9H-fluoro-9-yl)methyl((1S,10S)-5-cyclopropyl-10-ethyl-6-fluoro-10-hydroxy-11,14-dioxo-1,2,3,4,10,11,14,16-octahydro-13H-cyclohepta[3′,4′:6,7]indolizino[1,2-b]quinolin-1-yl)carbamate 23a

Compound 19j (500 mg, 0.72 mmol) was purified using a Gilson preparative instrument and a Daicel chiral column to separate chiral isomers (chromatographic column: CHIRALPAK IA 3.0 cm I.D.×25 cm, 10 μm; mobile phase 1: MeOH; mobile phase 2: DCM; gradient proportion: MeOH/DCM=70/30, flow rate: 25 mL/min) to give the compound 23a (220 mg, yield: 44%).

### Step II (1S,10S)-1-amino-5-cyclopropyl-10-ethyl-6-fluoro-10-hydroxy-2,3,4,10,13,16-hexahydro-14H-cyclohepta[3′,4′:6,7]indolizine[1,2-b]quinoline-11,14-(1H)-dione 23b

The compound 23a (45 mg, 0.064 mmol) was dissolved in a mixed solvent of N,N-dimethylformamide and diethylamine (7 mL, V/V=5:2), and the reaction mixture was stirred at room temperature for 2 h. After the reaction was complete, the reaction mixture was directly concentrated to give the crude compound 23b (30 mg), which was directly used in the next reaction step without purification.

MS m/z (ESI): 476.2 (M+1)⁺.

### Step III

### (R)-2-cyclopropyl-N-((1S,10S)-5-cyclopropyl-10-ethyl-6-fluoro-10-hydroxy-11,14-dioxo-1,2,3,4,10,11,14,16-octahydro-13H-cyclohepta[3′,4′:6,7]indolizino[1,2-b]quinolin-1-yl)-2-hydroxyacetamide

### (S)-2-cyclopropyl-N-((1S,10S)-5-cyclopropyl-10-ethyl-6-fluoro-10-hydroxy-11,14-dioxo-1,2,3,4,11,14,16-octahydro-13H-cyclohepta[3′,4′:6,7]indolizino[1,2-b]quinolin-1-yl)-2-hydroxyacetamide

The compound 23b (20 mg, 0.042 mmol) was dissolved in N,N-dimethylacetamide (2 mL), and 2-cyclopropyl-2-hydroxyacetic acid (5.4 mg, 0.046 mmol), (benzotriazol-1-yloxy)tris(dimethylamino)phosphonium hexafluorophosphate (22 mg, 0.051 mmol), and N,N-diisopropylethylamine (11 mg, 0.084 mmol) were added. The reaction mixture was stirred at room temperature for 3 h. After the reaction was complete, the reaction mixture was purified by preparative high performance liquid chromatography (Waters MS-triggered Prep-LC with QDA detector, chromatographic column: WELCH Xtimate C18 21.2×250 mm 10 μm; mobile phase 1: water (containing 0.1% FA); mobile phase 2: acetonitrile; 15-min gradient, gradient proportion: acetonitrile phase 46%-100%, flow rate: 30 mL/min) to give products 33-1 (4.1 mg, yield: 17.2%) and 33-2 (1.7 mg, yield: 6.9%).

### 33-1 (Monoconfigurational Compound, Short Retention Time)

MS m/z (ESI): 574.2 (M+1)⁺.

¹H NMR (400 MHz, DMSO-d₆) δ 8.62-8.37 (m, 2H), 7.69 (d, 1H), 7.28 (s, 1H), 6.53 (s, 1H), 5.59-5.48 (m, 2H), 5.43 (d, 2H), 5.28 (d, 1H), 3.62 (d, 1H), 3.49-3.46 (m, 1H), 2.19-1.72 (m, 6H), 1.69-1.53 (m, 1H), 1.23 (s, 1H), 1.18-1.00 (m, 3H), 0.85 (t, 3H), 0.83-0.77 (m, 1H), 0.74-0.62 (m, 1H), 0.46-0.28 (m, 4H).

### 33-2 (Monoconfigurational Compound, Long Retention Time)

MS m/z (ESI): 574.2 (M+1)⁺.

¹H NMR (400 MHz, DMSO-d₆) δ 8.45 (d, 2H), 7.69 (d, 1H), 7.28 (s, 1H), 6.53 (s, 1H), 5.60-5.52 (m, 1H), 5.48 (s, 1H), 5.43 (d, 2H), 5.30 (d, 1H), 3.59 (d, 1H), 3.49-3.46 (m, 1H), 2.14-1.79 (m, 6H), 1.75-1.61 (m, 1H), 1.24 (s, 1H), 1.17-1.02 (m, 3H), 0.86 (t, J=8 Hz, 3H), 0.83-0.77 (m, 1H), 0.74-0.64 (m, 1H), 0.48-0.29 (m, 4H).

### Example 18

### (S)-3-cyclopropyl-N-((1S,9S)-4-cyclopropyl-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)-3-hydroxypropanamide

### (R)-3-cyclopropyl-N-((1S,9S)-4-cyclopropyl-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)-3-hydroxypropanamide

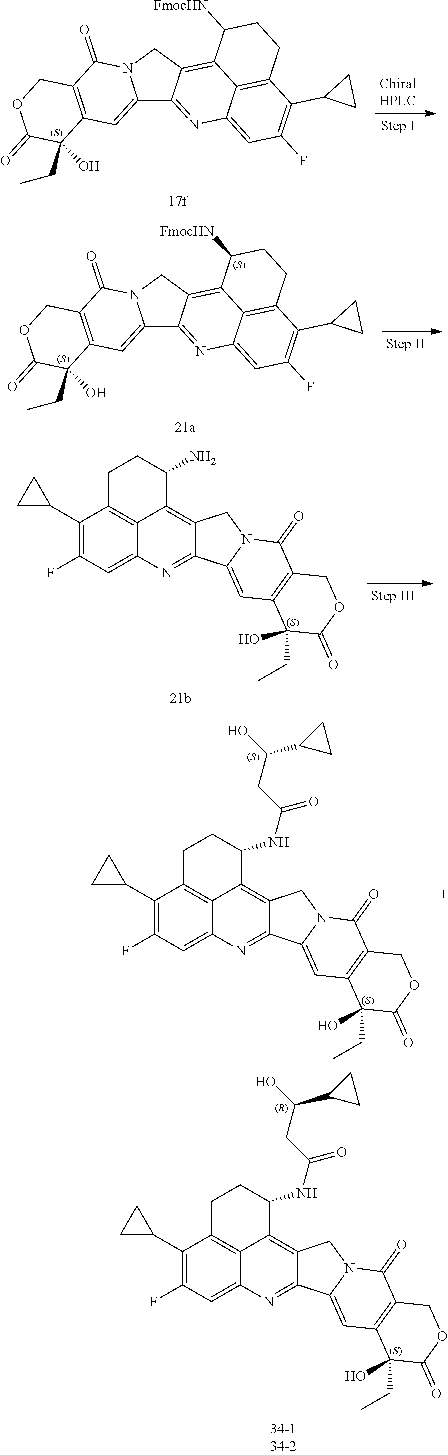

### Step I (9H-fluoren-9-yl)methyl((1S,9S)-4-cyclopropyl-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)carbamate 21a

The compound 17f (600 mg, 1.02 mmol) was purified using a Gilson preparative instrument and a Daicel chiral column to separate chiral isomers (chromatographic column: CHIRALPAK IB 3.0 cm I.D.×25 cm, 10 μm; mobile phase 1: MeOH; mobile phase 2: DCM; gradient proportion: MeOH/DCM=90/10, flow rate: 25 mL/min) to give the compound 21a (250 mg, yield: 42%).

### Step II (1S,9S)-1-amino-4-cyclopropyl-9-ethyl-5-fluoro-9-hydroxy-1,2,3,9,12,15-hexahydro-10H,13H-benzo[d]pyrano[3′,4′:6,7]indolizino[1,2-b]quinoline-10,13-dione 21b

The compound 21a (50 mg, 0.07 mmol) was dissolved in DMF (5 mL), and diethylamine (0.5 mL) was added. The mixture was stirred for reaction at room temperature for 1 h. After the reaction was complete, the mixture was spin-dried under vacuum to remove the solvent, thus giving the title compound 21b (34 mg), which was directly used in the next reaction step without purification.

MS m/z (ESI): 462.1 (M+1)⁺.

### Step III

### (S)-3-cyclopropyl-N-((1S,9S)-4-cyclopropyl-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)-3-hydroxypropanamide

### (R)-3-cyclopropyl-N-((1S,9S)-4-cyclopropyl-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)-3-hydroxypropanamide

The compound 21b (30 mg, 0.065 mmol) was dissolved in N,N-dimethylformamide (3 mL), and 3-cyclopropyl-3-hydroxypropionic acid (15 mg, 0.098 mmol), 2-(7-azabenzotriazole)-N,N,N′,N′-tramethyluronium hexafluorophosphate (37 mg, 0.096 mmol), and NN-diisopropylethylamine (171 mg, 1.32 mmol) were added. The reaction mixture was stirred at room temperature for 2 h. After the reaction was complete, the mixture was purified by preparative high performance liquid chromatography (Waters MS-triggered Prep-LC with QDA detector, chromatographic column: WELCH Xtimate C18 21.2×250 mm 10 μm; mobile phase 1: water (containing 0.1% FA); mobile phase 2: acetonitrile; 15-min gradient, gradient proportion: acetonitrile phase 40%-100%, flow rate: 30 mL/min) to give products 34-1 (4.2 mg, yield: 11%) and 34-2 (3.9 mg, yield: 10%).

34-1 (Monoconfigurational Compound, Compound with Short Retention Time)

MS m/z (ESI): 574.2 (M+1)⁺.

¹H NMR (400 MHz, DMSO-d₆) δ 8.53-8.39 (m, 2H), 7.75 (d, 1H), 7.30 (s, 1H), 6.53 (s, 1H), 5.55 (dd, 1H), 5.42 (s, 2H), 5.23 (s, 2H), 4.69 (s, 1H), 2.37 (d, 2H), 2.15 (d, 2H), 2.01-1.77 (m, 4H), 1.17-1.07 (m, 2H), 0.91-0.72 (m, 6H), 0.39-0.12 (m, 5H).

34-2 (Monoconfigurational Compound, Compound with Long Retention Time)

MS m/z (ESI): 574.2 (M+1)⁺.

1H NMR (400 MHz, DMSO-de) δ 8.45 (d, 2H), 7.75 (d, 1H), 7.31 (s, 1H), 6.53 (s, 1H), 5.64-5.54 (m, 1H), 5.43 (s, 2H), 5.24 (q, 2H), 4.65 (d, 1H), 2.33 (d, 2H), 2.27-1.73 (m, 6H), 1.12 (dd, 2H), 0.91-0.70 (m, 6H), 0.39-0.13 (m, 5H).

### Example 19

### (R)—N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxypentanamide

### (S)—N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxypentanamide

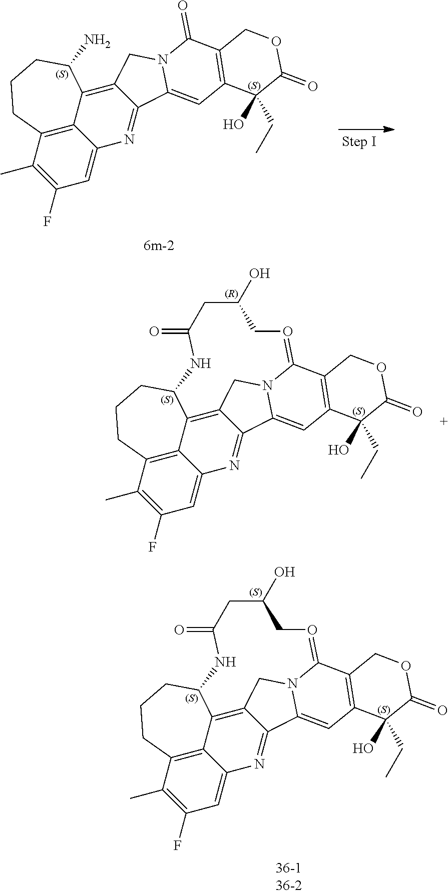

### Step I

### (R)—N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxypentanamide

### (S)—N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxypentanamide

The compound 6m-2 (30 mg, 0.07 mmol) was dissolved in N,N-dimethylformamide (3 mL), and then 3-hydroxypentanoic acid (9 mg, 0.07 mmol), N,N-diisopropylethylamine (17 mg, 0.13 mmol), and 2-(7-azabenzotriazole)-N,N,N′,N′-tramethyluronium hexafluorophosphate (38 mg, 0.10 mmol) were added. The reaction system was kept for reaction at normal temperature for 1 h. After the reaction was complete, the purification was performed by preparative high performance liquid chromatography (GILSON Prep LC with UV detector, chromatographic column: Xbridge 10 μm C18 250×30 mm, 10 μm; mobile phase 1: water (containing 10 mmol/LFA); mobile phase 2: acetonitrile; 15-min gradient, gradient proportion: acetonitrile phase 5%-57%, flow rate:50 mL/min) to give compounds 36-1(6.98 mg, yield: 18%) and 36-2 (6.70 mg, yield: 18%).

36-1 (Monoconfigurational Compound, Compound with Short Retention Time)

MS m/z (ESI): 550.2 (M+1)⁺.

¹H NMR (400 MHz, DMSO-d₆) δ 8.61 (d, 1H), 7.74 (d, 1H), 7.29 (s, 1H), 6.52 (s, 1H), 5.53 (s, 1H), 5.43 (s, 2H), 5.39-5.09 (m, 2H), 4.63 (s, 1H), 2.43 (s, 3H), 2.32 (d, 3H), 2.10-1.93 (m, 3H), 1.92-1.81 (m, 2H), 1.75 (s, 1H), 1.40-1.32 (m, 2H), 1.24 (s, 2H), 0.91-0.76 (m, 6H).

36-2 (Monoconfigurational Compound, Compound with Long Retention Time)

MS m/z (ESI): 550.2 (M+1)⁺.

¹H NMR (400 MHz, DMSO-d₆) δ 8.69 (d, 1H), 7.73 (d, 1H), 7.28 (s, 1H), 6.51 (s, 1H), 5.51 (d, 2H), 5.43 (s, 2H), 5.33 (d, 1H), 4.64 (s, 1H), 2.42 (s, 4H), 2.34-2.22 (m, 4H), 2.08 (d, 1H), 2.01 (d, 1H), 1.92-1.78 (m, 2H), 1.65 (s, 1H), 1.44-1.31 (m, 2H), 1.24 (s, 1H), 0.95-0.66 (m, 6H).

### Example 20

### (S)—N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxy-4-methylpentanamide

### (R)—N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxy-4-methylpentanamide

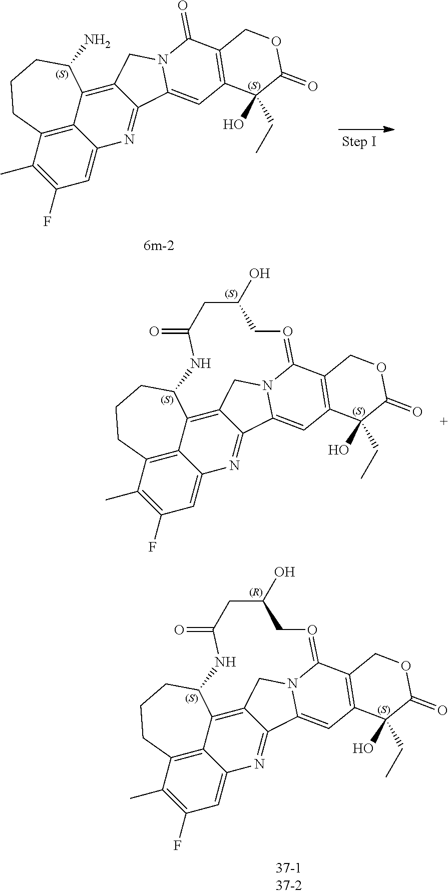

### Step I

### (S)—N-((1S,105)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxy-4-methylpentanamide

### (R)—N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxy-4-methylpentanamide

The compound 6m-2 (30 mg, 0.07 mmol) was dissolved in N,N-dimethylformamide (3 mL), and then 3-hydroxy-4-methylpentanoic acid (9 mg, 0.07 mmol), N,N-diisopropylethylamine (17 mg, 0.13 mmol), and 2-(7-azabenzotriazole)-N,N,N′,N′-tramethyluronium hexafluorophosphate (38 mg, 0.10 mmol) were added. The reaction system was kept for reaction at room temperature for 1 h. After the reaction was complete, the mixture was purified by preparative high performance liquid chromatography (Waters MS-triggered Prep-LC with SQD2 detector, chromatographic column: Xbridge 5 μm C18 150×19 mm; mobile phase 1: water (containing 0.1% FA); mobile phase 2: acetonitrile; 15-min gradient, gradient proportion: acetonitrile phase 42%-100%, flow rate: 20 mL/min) to give compound 37-1 (6.6 mg, yield: 18%) and 37-2 (6.9 mg, yield: 18%).

37-1 (Monoconfigurational Compound, Compound with Short Retention Time)

MS m/z (ESI): 564.2 (M+1).

¹H NMR (400 MHz, DMSO-d₆) δ 8.68 (d, 1H), 8.48 (br, 1H), 7.73 (d, 1H), 7.28 (s, 1H), 6.56 (s, 1H), 5.60-5.49 (m, 1H), 5.42 (s, 2H), 5.35 (d, 1H), 5.21 (d, 1H), 4.64 (s, 1H), 2.42 (s, 3H), 2.29 (d, 3H), 2.13-1.91 (m, 3H), 1.90-1.80 (m, 2H), 1.79-1.67 (m, 1H), 1.60-1.47 (m, 1H), 1.23 (s, 1H), 0.87 (d, 3H), 0.82 (d, 6H).

37-2 (Monoconfigurational Compound, Compound with Long Retention Time)

MS m/z (ESI): 564.2 (M+1)⁺.

¹H NMR (400 MHz, DMSO-d₆) δ 8.78 (s, 1H), 8.50 (s, 1H), 7.73 (s, 1H), 7.28 (s, 1H), 6.56 (s, 1H), 5.43 (s, 3H), 5.34 (d, 2H), 4.62 (s, 2H), 2.29 (s, 3H), 2.07 (s, 2H), 2.02-1.97 (m, 2H), 1.92-1.77 (m, 2H), 1.71-1.49 (m, 1H), 1.23 (s, 1H), 0.99-0.66 (m, 11H).

### Example 21

### (R)-3-cyclopropyl-N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxypropanamide

### (S)-3-cyclopropyl-N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxypropanamide

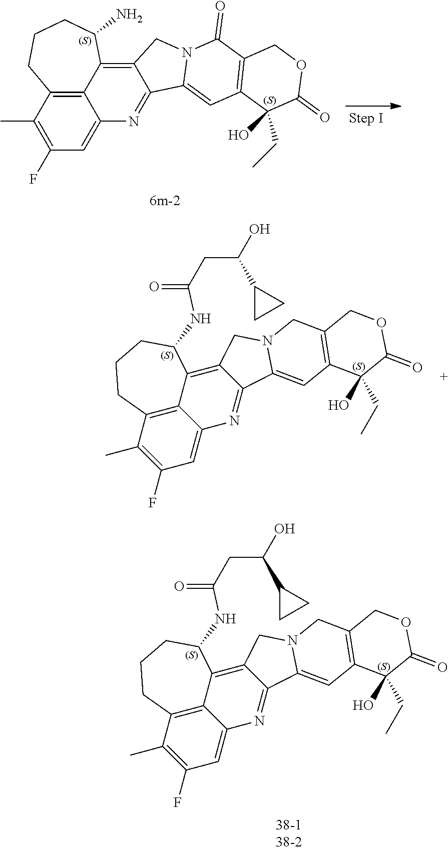

### (R)-3-cyclopropyl-N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxypropanamide

### (S)-3-cyclopropyl-N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxypropanamide

The compound 6m-2 (30 mg, 0.07 mmol) was dissolved in N,N-dimethylformamide (3 mL), and then 3-cyclopropyl-3-hydroxypropionic acid (18 mg, 0.14 mmol), N,N-diisopropylethylamine (17 mg, 0.13 mmol), and 2-(7-azabenzotriazole)-N,N,N′,N′-tramethyluronium hexafluorophosphate (38 mg, 0.10 mmol) were added. The reaction system was kept for reaction at normal temperature for 1 h. After the reaction was complete, the purification and separation were performed by preparative high performance liquid chromatography (Waters MS-triggered Prep-LC with QDA detector. chromatographic column: Xbridge 5 μm C18 150×19 mm; mobile phase 1: water (containing 0.1% FA); mobile phase 2: acetonitrile; 15-min gradient, gradient proportion: acetonitrile phase 45%-100%, flow rate: 25 mL/min) to give the title compound 38-1 (4 mg, yield: 11%) and the title compound 38-2 (4 mg, yield: 11%).

38-1 (Monoconfigurational Compound, Compound with Short Retention Time)

MS m/z (ESI):562.3 (M+1).

¹H NMR (400 MHz, DMSO-d₆) δ 8.63 (d, 1H), 7.74 (d, 1H), 7.28 (s, 1H), 6.52 (s, 1H), 5.53 (s, 1H), 5.42 (s, 4H), 4.69 (d, 1H), 3.22 (s, 2H), 2.61 (d, 1H), 2.42 (s, 3H), 2.31 (d, 1H), 2.08-1.69 (m, 6H), 0.87 (s, 3H), 0.81 (d, 1H), 0.37-0.20 (m, 3H), 0.16-0.08 (m, 1H).

38-2 (Monoconfigurational Compound, Compound with Long Retention Time)

MS m/z (ESI):562.3 (M+1)⁺.

¹H NMR (400 MHz, DMSO-d₆) δ 8.71 (d, 1H), 7.73 (d, 1H), 7.27 (s, 1H), 6.51 (s, 1H), 5.43 (s, 5H), 4.64 (d, 1H), 4.55-4.51 (m, 1H), 3.22 (s, 2H), 2.65 (d, 1H), 2.41 (s, 3H), 2.31 (d, 1H), 2.13-1.57 (m, 6H), 0.88 (s, 3H), 0.82 (s, 1H), 0.45 (s, 1H), 0.32-0.13 (m, 3H).

### Example 22

### (2S,3R)—N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxy-2-methylbutanamide 39

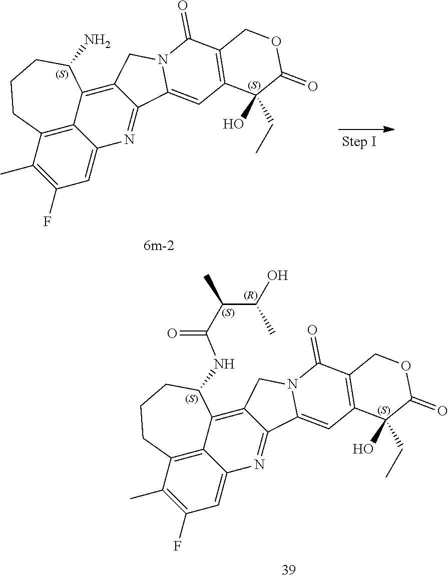

### Step I

### (2S,3R)—N-((1S,10S)-10-ethyl-6-fluoro-10-hydroxy-5-methyl-11,14-dioxo-1,2,3,4,10,11,14,16-octahydro-13H-cyclohepta[3′,4′:6,7]indolizino[1,2-b]quinolin-1-yl)-3-hydroxy-2-methylbutanamide 39

The compound 6m-2 (30 mg, 0.07 mmol) was dissolved in N,N-dimethylformamide (2 mL), and (2S,3R)-3-hydroxy-2-methylbutyric acid (12 mg, 0.10 mmol) and 2-(7-azabenzotriazole)-N,N,N′N′-tramethyluronium hexafluorophosphate (38 mg, 0.10 mmol) were added. The mixture was stirred for reaction at room temperature for 15 min. After the reaction was complete, the purification was performed by preparative high performance liquid chromatography (Waters MS-triggered Prep-LC with QDA detector, chromatographic column: Xbridge 5 μm C18 150×19 mm, 5 μm; mobile phase 1: water (containing 0.1% FA); mobile phase 2: acetonitrile; 10-min gradient, gradient proportion: acetonitrile phase 42%-52%, flow rate: 25 mL/min) to give the compound 39 (2 mg, yield: 5%).

MS m/z (ESI): 550.0 (M+1)⁺.

¹H NMR (400 MHz, CDCl₃) δ 7.73 (s, 1H), 7.42-7.28 (m, 2H), 5.51 (s, 1H), 5.41 (d, 1H), 5.35 (t, 1H), 5.26-5.16 (m, 2H), 5.04 (d, 1H), 4.18-4.00 (m, 2H), 3.33 (s, 2H), 2.65-2.50 (m, 3H), 2.48 (s, 3H), 2.27-2.18 (m, 1H), 2.11-2.04 (m, 2H), 2.03-1.97 (m, 1H), 1.33-1.28 (m, 6H), 0.95 (t, 3H).

### Biological Evaluation

The present invention is further described and interpreted below with reference to the test examples, but these test examples are not intended to limit the scope of the present invention.

### Test Example 1 Experiment of Inhibition of Compounds on Proliferation of Cells SK-BR-3

### 1.1 Experimental Materials

| Materials and reagents | Suppliers | Article numbers |
|---|---|---|
| SK-BR-3 | ATCC | HTB-30 |
| 5A Medium | Gibco | 16600-082 |
| Penicillin-streptomycin | Gibco | 15140-122 |
| Fetal bovine serum | Gibco | 10099-141C |
| Phosphate buffer saline | Gibco | 10010-031 |
| TrypLE | Gibco | 12604-021 |
| DMSO | Sigma | D8418-1L |
| Staurosporine | MCE | HY-15141 |
| CelltiterGlo assay kit (CTG) | Promega | G7573 |

### 1.2 Experimental Instruments:

| | | Article |
|---|---|---|
| Consumables and instruments | Suppliers | numbers |
| 384-well white culture plate | PerkinElmer | 6007680 |
| Platform rocker | QILINBEIER | QB-9002 |
| Centrifuge | Eppendorf | 5810R |
| Carbon dioxide incubator | Thermo Scientific | 371 |
| Microscope | OLYMPUS | CKX41 |
| Echo Qualified 384-Well | LABCYTE | PP-0200 |
| Polypropylene | | |
| Echo | LABCYTE | 655 |
| CountessII | Gibco | AMQAX1000 |
| Envision | PerkinElmer | 2105 |

### 1.3 Test Method

(1) Cell plating: first, tumor cells SK-BR-3 were cultured using a corresponding culture medium, trypsinized, centrifuged, resuspended for counting, adjusted to an appropriate concentration, and then plated on a 384-well plate.
(2) Co-incubation of the compounds and tumor cells: after the cells adhered to the wall, 100 nL of a diluted bioactive substance (test compound) was added to the cell culture plate using ECHO, the final concentration of DMSO in wells of the cell plate was 0.33%, and the incubation was performed in an incubator at 37° C. with 5% CO₂ for 72 h.
(3) After the incubation was complete, 30 μL of a CTG reagent (CelltiterGlo kit) was added to each well. The culture was oscillated on a fast oscillator for 2 min, centrifuged at 1,000 rpm for 1 min, and kept away from light at room temperature for 30 min. The chemiluminescence signal value was read using an Envision instrument.
(4) Cell viability detection: IC₅₀ was calculated using GraphPad Prism 8 software, and the IC₅₀ of the compounds was obtained using the following nonlinear fitting formula (see Table 1):

$$Y = Bottom + ( Top - Bottom ) / ( 1 + 10 \hat{} ( ( Log {IC}_{50} - X ) * HillSlope ) )$$

Y: inhibition rate; X: log value of compound concentration-,

$$inhibition rate ( \% ) = 100 - ( compound well reading - low - reading control well reading ) / ( high - reading control well reading - low - reading control well reading ) * 100 ;$$

high-reading control wells: cells with addition of 100 nL DMSO; and
low-reading control wells: cell-free wells.

TABLE 1
Antiproliferative activity of test compounds on SK-BR-3

| Example and Compound No. | IC₅₀ (nM) |
|---|---|
| Control compound (Dxd) | 11 |
| 3-1 | 13 |
| 3-2 | 15 |
| 4-1 | 61 |
| 4-2 | 14 |
| 5-1 | 20 |
| 5-2 | 20 |
| 6-1 | 11 |
| 6-2 | 7.3 |
| 7-1 | 8.6 |
| 7-2 | 7.6 |
| 8-1 | 44 |
| 8-2 | 87 |
| 10 | 10 |
| 11-1 | 2.2 |
| 11-2 | 2.4 |
| 12 | 5.7 |
| 17-1 | 4.6 |
| 17-2 | 6.6 |
| 18-1 | 13.8 |
| 18-2 | 9.6 |
| 19-1 | 8.3 |
| 19-2 | 7.6 |
| 20-1 | 21 |
| 20-2 | 25 |
| 33-1 | 7.8 |
| 33-2 | 8.4 |
| 34-1 | 5.1 |
| 34-2 | 4.8 |
| 36-1 | 9.2 |
| 36-2 | 4.4 |
| 37-1 | 19 |
| 37-2 | 6.6 |
| 38-1 | 12 |
| 38-2 | 5.4 |
| 39 | 11 |

The result shows that the compounds of the present invention have strong antiproliferative effects on the proliferation of cells SK-BR-3.

### Test Example 2 Experiment of Inhibition of Test Compounds on Proliferation of Cells MDA-MB-468

### 2.1 Experimental Materials

| Materials and reagents | Suppliers | Article numbers |
|---|---|---|
| MDA-MB-468 | ATCC | HTB-132 |
| L-15 Medium | Hyclone | SH30525.01 |
| Penicillin-streptomycin | Gibco | 15140-122 |
| Fetal bovine serum | Gibco | 10099-141C |
| Phosphate buffer saline | Gibco | 10010-031 |
| TrypLE | Gibco | 12604-021 |
| DMSO | Sigma | D8418-1L |
| Staurosporine | MCE | HY-15141 |
| CelltiterGlo assay kit (CTG) | Promega | G7573 |

### 2.2 Experimental Instruments:

| | | Article |
|---|---|---|
| Consumables and instruments | Suppliers | numbers |
| 384-well white culture plate | PerkinElmer | 6007680 |
| Platform rocker | QILINBEIER | QB-9002 |
| Centrifuge | Eppendorf | 5810R |
| Carbon dioxide incubator | Thermo Scientific | 371 |
| Microscope | OLYMPUS | CKX41 |
| Echo Qualified 384-Well | LABCYTE | PP-0200 |
| Polypropylene | | |
| Echo | LABCYTE | 655 |
| CountessII | Gibco | AMQAX1000 |
| Envision | PerkinElmer | 2105 |

### 2.3 Test Method

(1) Cell plating: first, tumor cells MDA-MB-468 were cultured using a corresponding culture medium, trypsinized, centrifuged, resuspended for counting, adjusted to an appropriate concentration, and then plated on a 384-well plate.
(2) Co-incubation of the compounds and tumor cells: after the cells adhered to the wall, 100 nL of a diluted bioactive substance (test compound) was added to the cell culture plate using ECHO, the final concentration of DMSO in wells of the cell plate was 0.33%, and the incubation was performed in an incubator at 37° C. with 0% CO₂ for 72 h.
(3) After the incubation was complete, 30 μL of a CTG reagent (CelltiterGlo kit) was added to each well. The culture was oscillated on a fast oscillator for 2 min, centrifuged at 1,000 rpm for 1 min, and kept away from light at room temperature for 30 min. The chemiluminescence signal value was read using an Envision instrument.
(4) Cell viability detection: IC₅₀ was calculated using GraphPad Prism 8 software, and the IC₅₀ of the compounds was obtained using the following nonlinear fitting formula (see Table 2):

$$Y = Bottom + ( Top - Bottom ) / ( 1 + 10 \hat{} ( ( Log {IC}_{50} - X ) * HillSlope ) )$$

Y: inhibition rate: X: log value of compound concentration;

$$inhibition rate ( \% ) = 100 - ( compound well reading - low - reading control well reading ) / ( high - reading control well reading - low - reading control well reading ) * 100 ;$$

high-reading control wells: cells with addition of 100 nL DMSO; and
low-reading control wells: cell-free wells.

TABLE 2
Antiproliferative activity of test compounds on MDA-MB-468

| Example and Compound No. | IC₅₀ (nM) |
|---|---|
| Control compound (Dxd) | 4.6 |
| 3-1 | 7.5 |
| 3-2 | 12 |
| 4-1 | 34 |
| 4-2 | 13 |
| 5-1 | 6.7 |
| 5-2 | 9.4 |
| 6-1 | 8.8 |
| 6-2 | 6.5 |
| 7-1 | 3.1 |
| 7-2 | 4.4 |
| 8-1 | 13 |
| 8-2 | 38 |
| 10 | 13 |
| 11-1 | 3.9 |
| 11-2 | 4.5 |
| 12 | 9.3 |
| 17-1 | 5.8 |
| 17-2 | 8.1 |
| 18-1 | 12 |
| 18-2 | 12 |
| 19-1 | 7.1 |
| 19-2 | 6.9 |
| 20-1 | 15.6 |
| 20-2 | 19.5 |
| 33-1 | 6 |
| 33-2 | 5 |
| 34-1 | 4 |
| 34-2 | 4 |
| 36-1 | 14 |
| 36-2 | 6.4 |
| 37-1 | 19 |
| 37-2 | 6.6 |
| 38-1 | 13 |
| 38-2 | 5.7 |
| 39 | 15 |

The result shows that the test compounds have strong antiproliferative effects on the proliferation of cells MDA-MB-468.

### Test Example 3 Experiment of Inhibition of Test Compounds on Proliferation of Cells NCI-N87

### 3.1 Experimental Materials

| Materials and reagents | Suppliers | Article numbers |
|---|---|---|
| NCI-N87 | ATCC | HTB-30 |
| RPMI-1640 Medium | Gibco | A10491-01 |
| Penicillin-streptomycin | Gibco | 15140-122 |
| Fetal bovine serum | Gibco | 10099-141C |
| Phosphate buffer saline | Gibco | 10010-031 |
| TrypLE | Gibco | 12604-021 |
| DMSO | Sigma | D8418-1L |
| Staurosporine | MCE | HY-15141 |
| CelltiterGlo assay kit (CTG) | Promega | G7573 |

### 3.2 Experimental Instruments:

| | | Article |
|---|---|---|
| Consumables and instruments | Suppliers | numbers |
| 384-well white culture plate | PerkinElmer | 6007680 |
| Platform rocker | QILINBEIER | QB-9002 |
| Centrifuge | Eppendorf | 5810R |
| Carbon dioxide incubator | Thermo Scientific | 371 |
| Microscope | OLYMPUS | CKX41 |
| Echo Qualified 384-Well | LABCYTE | PP-0200 |
| Polypropylene | | |
| Echo | LABCYTE | 655 |
| CountessII | Gibco | AMQAX1000 |
| Envision | PerkinElmer | 2105 |

### 3.3 Test Method

(1) Cell plating: first, tumor cells NCI-N87 were cultured using a corresponding culture medium, trypsinized, centrifuged, resuspended for counting, adjusted to an appropriate concentration, and then plated on a 384-well plate.
(2) Co-incubation of the compounds and tumor cells: after the cells adhered to the wall, 100 nL of a diluted bioactive substance (test compound) was added to the cell culture plate using ECHO, the final concentration of DMSO in wells of the cell plate was 0.33%, and the incubation was performed in an incubator at 37° C. with 0% CO₂ for 72 h.
(3) After the incubation was complete, 30 μL of a CTG reagent (CelltiterGlo kit) was added to each well. The culture was oscillated on a fast oscillator for 2 min, centrifuged at 1,000 rpm for 1 min, and kept away from light at room temperature for 30 min. The chemiluminescence signal value was read using an Envision instrument.
(4) Cell viability detection: IC₅₀ was calculated using GraphPad Prism 8 software, and the IC₅₀ of the compounds was obtained using the following nonlinear fitting formula (see Table 3):

$$Y = Bottom + ( Top - Bottom ) / ( 1 + 10 \hat{} ( ( Log {IC}_{50} - X ) * HillSlope ) )$$

Y: inhibition rate, X: log value of compound concentration;

$$inhibition rate ( \% ) = 100 - ( compound well reading - low - reading control well reading ) / ( high - reading control well reading - low - reading control well reading ) * 100 ;$$

high-reading control wells: cells with addition of 100 nL DMSO; and
low-reading control wells: cell-free wells.

TABLE 3
Antiproliferative activity of test compounds on cells NCI-N87

| Example and Compound No. | IC₅₀ (nM) |
|---|---|
| Control compound (Dxd) | 11 |
| 3-1 | 16 |
| 3-2 | 16 |
| 4-2 | 22 |
| 5-1 | 14 |
| 5-2 | 18 |
| 6-1 | 7.5 |
| 6-2 | 6.8 |
| 7-2 | 4.9 |
| 8-1 | 4.2 |
| 9 | 3 |
| 10 | 23 |
| 11-1 | 2.5 |
| 11-2 | 2.3 |
| 12 | 20 |
| 17-1 | 3.6 |
| 17-2 | 5.3 |
| 18-1 | 11.2 |
| 18-2 | 16.5 |
| 19-1 | 7.4 |
| 19-2 | 7.4 |
| 20-1 | 26 |
| 20-2 | 28 |
| 33-1 | 8 |
| 33-2 | 9 |
| 34-1 | 6 |
| 34-2 | 6 |
| 36-1 | 8.5 |
| 36-2 | 3.9 |
| 37-1 | 8.6 |
| 37-2 | 3.2 |
| 38-1 | 7.5 |
| 38-2 | 2.9 |
| 39 | 9.3 |

The result shows that the test compounds have strong anti proliferative effects on the proliferation of cells NCI-N87.

### Test Example 4 Kinetic Solubility Test in PBS 7.4

### 4.1. Experimental Steps

### 1) Preparation of Stock Solutions

10 mM stock solutions of the test substances 6-2, 11-2, 17-2, Dxd, and the control drugs progesterone and diclofenac were prepared using DMSO respectively.

### 2) Kinetic Solubility Test Steps

15 μL of 10 mM stock solution of each of the test substances was added to a corresponding position of a corresponding 96-well plate in a specified sequence. 485 μL of the PBS 7.4 was added to a corresponding vial on a sample plate. The experiment was run in duplicate. Each vial was additionally provided with a stirring rod and was plugged. Then, the sample plate was transferred in a thermostatic mixer, and oscillated at a revolving speed of 1,100 rpm at 25° C. for 2 h. 2 h later, the plug was removed, the stirring rod was attracted away with a large magnet, and then the sample was transferred from the sample plate to the filter plate. Negative pressure was generated using a vacuum pump to filter the sample. 5 μL of filtrate and 5 μL of blank DMSO were transferred to another sample plate, and then 490 μL of internal standard water (acetonitrile:water=1:1) containing the internal standard was added. Depending on peak shapes, a certain proportion of internal standard water may be used to dilute the sample diluent to obtain better peak shapes.

### 3) Preparation of 3 μM of Standard Substance Solution

6 μL was transferred from the 10 mM DMSO stock solution plate to a blank plate, and was prepared into a 300 μM standard substance solution with addition of 194 μL of DMSO. 5 μL was transferred from the 300 μM standard substance solution plate to still another blank plate, and then 5 μL of blank buffer and 490 μL of the internal standard water containing the internal standard (acetonitrile:water=1:1) were added until a final concentration of the standard substance solution was 3 μM.

### 4) Sample Analysis Steps

An injection plate was transferred on an injection tray of an autosampler to evaluate the sample by LC/MS analysis.

### 4.0. Data Analysis

All calculations were performed using Microsoft Excel.

The sample filtrate was analyzed and quantified by qualitative and quantitative LC/MS of standard substance peaks at known concentrations. The calculation formula for the solubility values of the reference drugs and the test substances are as follows:

$$[ Sample ] = \frac{{AREA}_{Sample} \times {DF}_{Sample} \times [ STD ]}{{AREA}_{Std}}$$

[Sample] is the compound concentration, DF_{Sample} refers to the sample dilution factor, [STD] is the standard substance concentration of the compound, AREA_{Std} is the standard substance peak area of the compound, and AREA_{Sample} is the peak area of the compound sample.

### 4.2 Experimental Result

The experimental result is as shown in Table 4.

TABLE 4
Solubility of representative compounds in PBS 7.4

| Compound | 6-2 | 11-2 | 17-2 | Dxd |
|---|---|---|---|---|
| Solubility in PBS 7.4 (μM) | 170 | 34 | 1.7 | 11.3 |

The result shows that the representative compounds 6-2 and 11-2 have significantly better solubility in the PBS 7.4 than Dxd.

### Test Example 5 Pharmacokinetic Test in SD Rats

After a single intravenous injection (dose: 2 mpk) of the compounds 6-2, 11-2, 17-2, and Dxd into male SD rats (blood collection time points: 0.083 h, 0.25 h, 0.5 h, 1 h, 2 h, 4 h, 8 h, and 24 h after IV administration, totaling 8 blood collection time points), average pharmacokinetic parameters in the plasma are as shown in Table 5:

TABLE 5
Pharmacokinetic test result of representative compounds

| | | | | | CL | |
|---|---|---|---|---|---|---|
| Com- | C₀ | AUCₗₐₛₜ | AUC_{inf} | T_{1/2} | (mL/min/ | Vₛₛ |
| pound | (ng/mL) | (h*ng/mL) | (h*ng/mL) | (h) | kg) | (L/kg) |
| 6-2 | 677 | 314 | 318 | 0.39 | 107.9 | 2.93 |
| 11-2 | 848 | 460 | 465 | 0.63 | 72.7 | 3.01 |
| 17-2 | 2693 | 2253 | 2734 | 1.64 | 12.32 | 1.4 |
| Dxd | 748 | 301 | 302 | 0.39 | 113 | 2.70 |

The result shows that the representative compounds of the present application are rapidly removed from the rats, have good safety, and have good pharmacokinetic properties.

### Test Example 6 Experiment of Inhibition of Compounds on Human Hepatomicrosomes CYP1A2, CYP2D36, and CYP3A4

### 6.1 Positive Control Inhibitors of Each Subtype

| CYP | Inhibitor | Final concentration (μM) |
|---|---|---|
| 1A2 | Furafylline | 0, 0.1, 0.3, 1, 3, 10, 30 |
| 2D6 | Quinidine | 0, 0.0015, 0.005, 0.015, 0.05, 0.15, 0.5 |
| 3A4 | Ketoconazole | 0, 0.0015, 0.005, 0.015, 0.05, 0.15, 0.5 |

### 6.2 Preparation of Substrate Stock Solutions

| | | | | Final | Incu- |
|---|---|---|---|---|---|
| | | Working | | substrate | bation |
| | Labeled | concentration | | concentration | time |
| CYP | substrate | (μM) | | (μM) | (min) |
| 1A2 | Phenacetin | 800 | (100 mM PBS) | 40 | 20 |
| 2D6 | Dextro- | 40 | (100 mM PBS) | 2 | 20 |
| | methorphan | | | | |
| 3A4M | Midazolam | 20 | (100 mM PBS) | 1 | 5 |
| 3A4T | Testosterone | 80 | (100 mM PBS) | 40 | 10 |

### 6.3 Experimental Process

The incubation was performed in a 96-well deep well plate. The following volumes were added to each well of the incubation plate: 169 μL of “master solution” and 1 μL of each of the test compounds or positive control compound (DMSO) at various concentrations. The incubation plate was transferred to a water bath for pre-incubation at 37° C. for 5 min. Then, 10 μL of diluted substrate solution was added to the incubation plate, and mixed and incubated on a vortex mixer for 15 sec. Then, 20 μL of 10 mM NADPH solution was added, and the reaction started from the final concentration of 1 mM. At a predetermined time point, 300 μL of a quenching solution (cold acetonitrile with addition of 3% formic acid, 200 nM alprazolam, 200 nM labetalol hydrochloride, and 200 nM tolbutamide) was added to quench the reaction. The mixture was centrifuged at 3,220 g for 40 min. 150 μL of the supernatant was transferred to another plate. The supernatant can be diluted with 150 μL of pure water. The mixture was fully mixed, and the content of substrate metabolites was determined by LC/MS/MS.

### 6.4 Data Analysis

Automatic peak integration domains were checked for all samples. The peak areas and internal standard peak areas were analyzed, and then exported to an excel spreadsheet. Inhibition of each enzyme P450 in human hepatomicrosomes was measured as the percentage decrease of marker metabolite-forming activity, as compared to non-inhibited controls (=100% activity). The IC₅₀ values were calculated as the logarithm of residual activity (%) and inhibitor concentration.

The percentage of the residual activity was calculated as follows:

$$\begin{matrix}Area ratio = analyte peak area / internal standard peak area \\ Residual activity ( \% ) = area {ratio}_{test drug} / area {ratio}_{blank control} * 100 \%\end{matrix}$$

The IC₅₀ value was calculated using Excel XLfit 5.5.1.3.

The experimental result is as shown in Table 6.

TABLE 6
Experimental result of inhibition of representative
compounds on human hepatomicrosomes

| | 1A2 | 2D6 | 3A4T | 3A4M |
|---|---|---|---|---|
| Compound | (IC₅₀ μM) | (IC₅₀ μM) | (IC₅₀ μM) | (IC₅₀ μM) |
| 6-2 | >30 | >30 | >30 | >30 |
| 11-2 | >30 | >30 | >30 | >30 |
| 17-2 | >30 | >30 | >30 | >30 |
| Dxd | >30 | >30 | >30 | >30 |

The result shows that the representative compounds of the present application have weak inhibitory effect on CYP enzymes and have good safety.

### Test Example 7 Test of Stability of Compounds in Human Plasma

### 7.1 Experimental Process

(1) 199 μL of human plasma was added to the culture plate of each cell. The culture plate was preheated to 37° C., and kept at this temperature for 15 min.
(2) After preincubation, 1 μL of 1 mmol/L each test compound and 1 μL of 1 mmol/L control compound were added to 199 μL of plasma, so that a final concentration of each test compound was 5 μmol/L and that of the control compound was 5 gmol/L. The final concentration of the organic solvent was 0.5%. The experiment would be duplicated twice.
(3) Reaction samples were incubated at 37° C.
(4) The reaction was stopped by adding 600 μL of cold methanol containing the internal standard at 0, 1, 2, 6, and 24 h. Each sample was vortex mixed for 10 min, and then centrifuged at 3,220 g for 30 min to precipitate the protein. 100 μL of the supernatant was transferred to another plate. Based on the signal response and peak shapes of liquid chromatography-mass spectrometry (LC-MS), the supernatant would be diluted with ultrapure water.

### 7.2 Data Analysis

All calculations were performed using Microsoft Excel. The peak area ratio was determined from the chromatogram of extracted ions. The percentage of compounds remaining at each time point was calculated as per the following formula:

$$Residual {percentage}_{tmin} ( \% ) = peak area {ratio}_{tmin} / peak {area}_{t 0} \times 100$$

The experimental result is as shown in Table 7.

TABLE 7
Test result of stability of representative
compounds in human plasma

| | Residual percentage (%) | | | | | |
|---|---|---|---|---|---|---|
| Compound | 0 h | 1 h | 2 h | 4 h | 6 h | 24 h |
| 6-2 | 100.00 | 96.62 | 86.16 | 93.65 | 88.99 | 87.01 |
| 11-2 | 100.00 | 96.80 | 92.06 | 93.40 | 94.45 | 88.81 |
| 17-2 | 100.00 | 90.07 | 89.34 | 93.75 | 97.06 | 102.21 |
| Dxd | 100.00 | 89.43 | 86.80 | 83.58 | 86.42 | 82.33 |

The result shows that the representative compounds 6-2, 11-2, and 17-2 of the present application have good stability in human plasma.

The embodiments of the technical solutions of the present invention are illustratively described above. It is to be understood that the scope of protection of the present invention is not limited to the above-mentioned embodiments. Any modification, equivalent replacement, improvement, or the like made by those skilled in the art within the spirit and principle of the present invention should be encompassed within the scope of protection of the claims of the present application.

1. A compound represented by formula I, a racemate, a stereoisomer, a tautomer, an isotopically labeled compound, a solvate, a polymorph, a pharmaceutically acceptable salt, or a prodrug compound thereof: 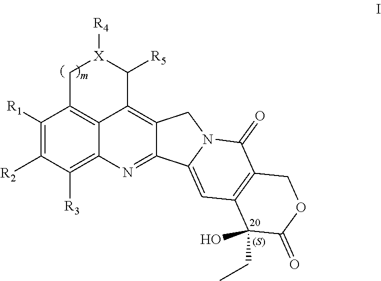
wherein R₁, R₂, and R₃ are identical or different, and are selected independently of each other from H, OH, CN, a halogen, a C₁₋₁₀ alkyl, a C₂₋₁₀ alkenyl, a C₂₋₁₀ alkynyl, a C₁₋₁₀ alkoxy, a C₁₋₁₀ haloalkyl, a C₁₋₁₀ haloalkoxy, a C₁₋₁₀ cyanoalkyl, a C₁₋₁₀ cyanoalkoxy, or a C₃₋₁₀ cycloalkyl;
R₄ is selected from H or 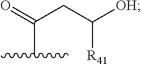
R₄₁ is selected from H, a C₁₋₆ alkyl, a C₁₋₆ haloalkyl, a C₁₋₆ alkyl-NH—, a (C₁₋₆ alkyl)₂N—, a C₁₋₆ alkyl-NH—C₁₋₆ alkyl, a (C₁₋₆ alkyl)₂N—C₁₋₆ alkyl, a C₁₋₆ alkoxyalkyl, a C₂₋₆ alkenyl, a C₂₋₆ alkynyl, a C₃₋₆ cycloalkyl, a 3-6-membered heterocyclyl, a C₆₋₁₄ aryl, and a 5-14-membered heteroaryl;
R₅ is selected from H, 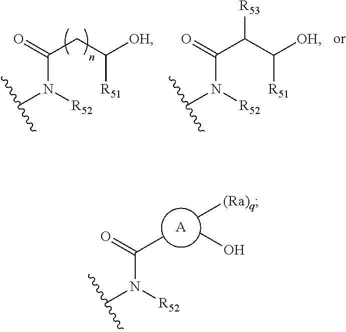
R₅₁ and R₅₂ are identical or different, and are selected independently of each other from H, a C₁₋₆ alkyl, a C₁₋₆ haloalkyl, a C₁₋₆ alkyl-NH—, a (C₁₋₆ alkyl)₂N—, a C₁₋₆ alkyl-NH—C₁₋₆ alkyl, a (C₁₋₆ alkyl)₂N—C₁₋₆ alkyl, a C₁₋₆ alkoxyalkyl, a C₂₋₆ alkenyl, a C₂₋₆ alkynyl, a C₃₋₆ cycloalkyl, a 3-6-membered heterocyclyl, a C₆₋₁₄ aryl, and a 5-14-membered heteroaryl; R₅₃ is selected from a C₁₋₆ alkyl, a C₁₋₆ haloalkyl, a C₁₋₆ alkyl-NH—, a (C₁₋₆ alkyl)₂N—, a C₁₋₆ alkyl-NH—C₁₋₆ alkyl, a (C₁₋₆ alkyl)₂N—C₁₋₆ alkyl, a C₁₋₆ alkoxyalkyl, a C₂₋₆ alkenyl, a C₂₋₆ alkynyl, a C₃₋₆ cycloalkyl, a 3-6-membered heterocyclyl, a C₆₋₁₄ aryl, and a 5-14-membered heteroaryl; ring A is selected from a C₃₋₈ cycloalkyl or a C₃₋₈ heterocyclyl, Ra is selected from H, hydroxyl, CN, a halogen, a C₁₋₆ alkyl, or a C₁₋₆ haloalkyl; n is selected from 0, 1, or 2; and q is selected from 0, 1, or 2;
X is selected from CH or N; and
m is an integer selected from 0-6.

2. The compound according to claim 1, wherein the R₁ is selected from H, OH, CN, a halogen, a C₁₋₆ alkyl, a C₂₋₆ alkenyl, a C₂₋₆ alkynyl, a C₃₋₆ cycloalkyl, or a C₁₋₆ haloalkoxy;
preferably, the R₁ is selected from H, OH, CN, a halogen, a C₁₋₆ alkyl, a C₂₋₆ alkenyl, a C₂₋₆ alkynyl, or a C₁₋₆ haloalkoxy;
preferably, the R₁ is selected from H, OH, Br, methyl, difluoromethoxy, 2,2,2-trifluoroethoxy, ethenyl, cyclopropyl, or ethynyl;
preferably, the R₁ is selected from H, OH, Br, methyl, difluoromethoxy, 2,2,2-trifluoroethoxy, ethenyl, or ethynyl;
preferably, the R₂ is selected from H, a halogen, CN, or a C₁₋₆ alkyl;
preferably, the R₂ is selected from H or F;
preferably, the R₃ is selected from H or a C₁₋₆ alkyl; and
preferably, the R₃ is H.

3. The compound according to claim 1, wherein the R₄ is selected from H or 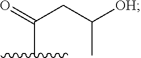 [SMILES: *C(=O)CC(C)O]
for example, 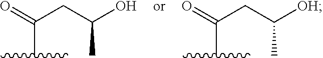
preferably, the R₄ is selected from H or 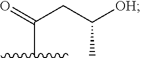 [SMILES: *C(=O)C[C@@H](C)O]
preferably, the X—R₄ is 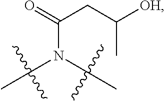 [SMILES: *N(*)C(=O)CC(C)O]
and is preferably 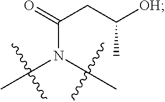 [SMILES: *N(*)C(=O)C[C@@H](C)O]
and
preferably, the X—R₄ is —CH₂—.

4. The compound according to claim 1, wherein the R₅ is selected from H, 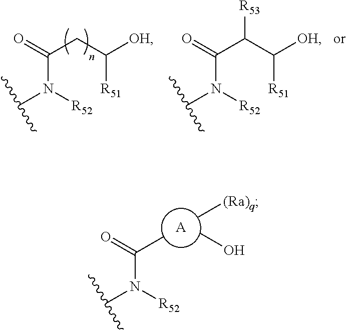
wherein the R₅₁ is selected from H, methyl, ethyl, isopropyl, or cyclopropyl; the R₅₂ is selected from H or methyl; the R₅₃ is selected from methyl; the ring A is selected from a C₃₋₆ cycloalkyl; the Ra is selected from H, hydroxyl, CN, a halogen, a C₁₋₆ alkyl, or a C₁₋₆ haloalkyl; the n is selected from 0 or 1; and the q is selected from 0 or 1;
preferably, the ring A is selected from a cyclobutane ring;
wherein the R₅ is selected from H, 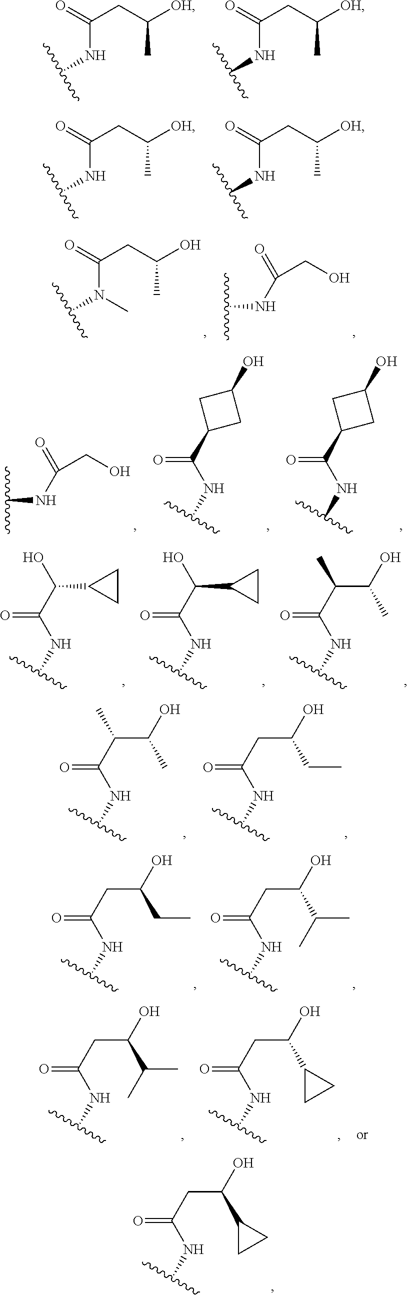
preferably, the R₅ is selected from H, 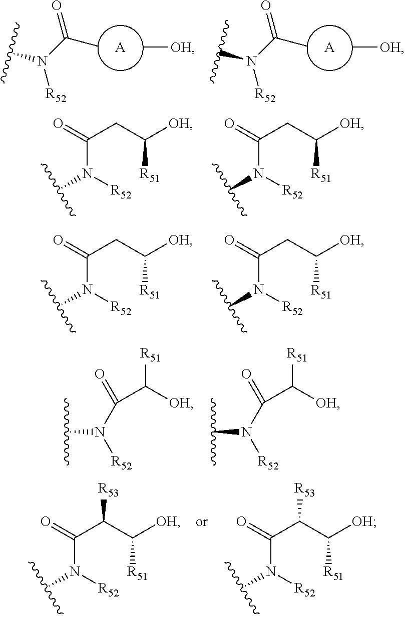
preferably, the R₅₁ is selected from H, methyl, ethyl, isopropyl, or cyclopropyl; the R₅₂ is selected from H or methyl; the R₅₃ is selected from methyl; and the ring A is selected from a cyclobutane ring; and
preferably, the R₅ is selected from H,

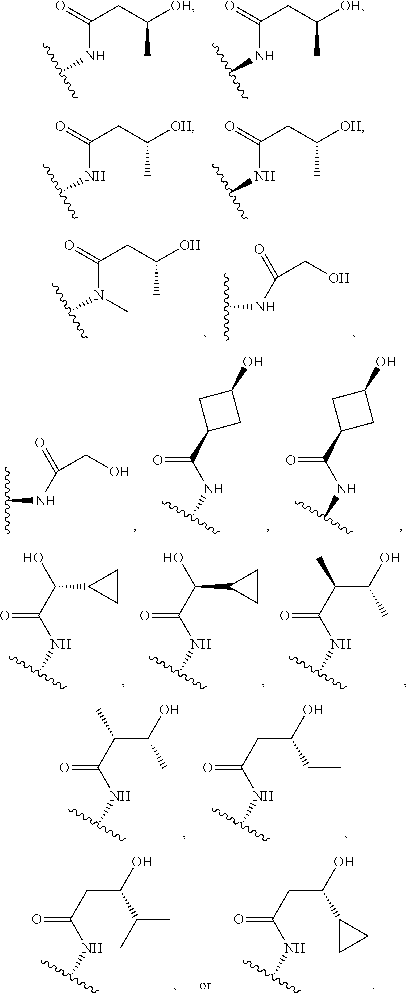

5. The compound according to claim 1, wherein the X is selected from CH or N; and when X is CH, the R₄ is H; or when X is N, the R₅ is H; and
preferably, the m is selected from 0, 1, or 2.

6. The compound according to claim 1, wherein the compound represented by formula I has a structure as shown below: 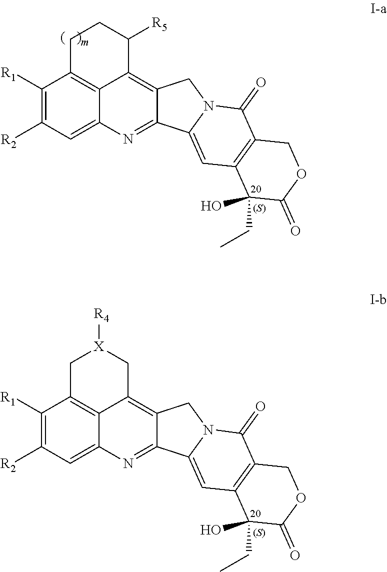
wherein the R₁, R₂, R₄, R₅, X, and m are as defined in claim 1;
preferably, the compound represented by formula I has a structure as shown below: 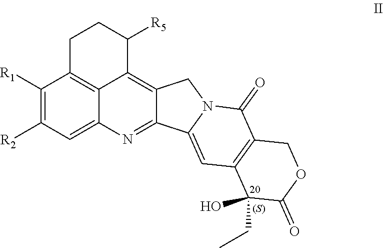
wherein the R₁, R₂, and R₅ are as defined in claim 1; and
preferably, the compound represented by formula I has a structure as shown below: 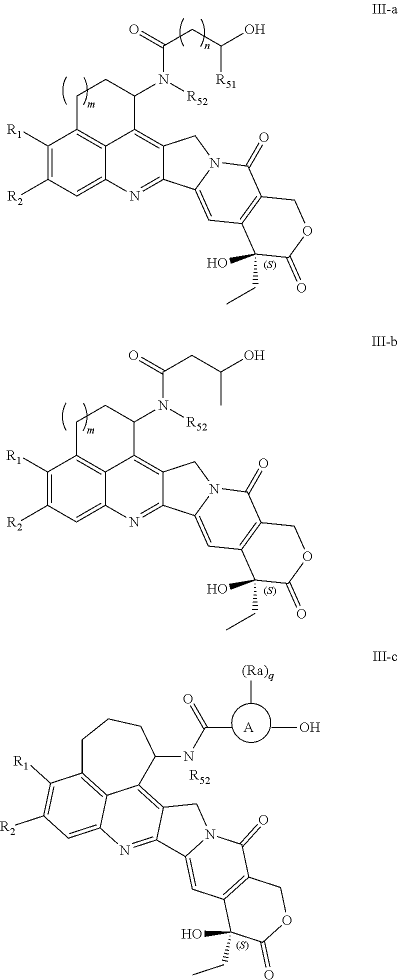
wherein the R₁, R₂, R₅₁, R₅₂, ring A, Ra, m, n, and q are as defined in claim 1.

7. The compound according to claim 1, wherein the compound of formula I has a structure as shown below:

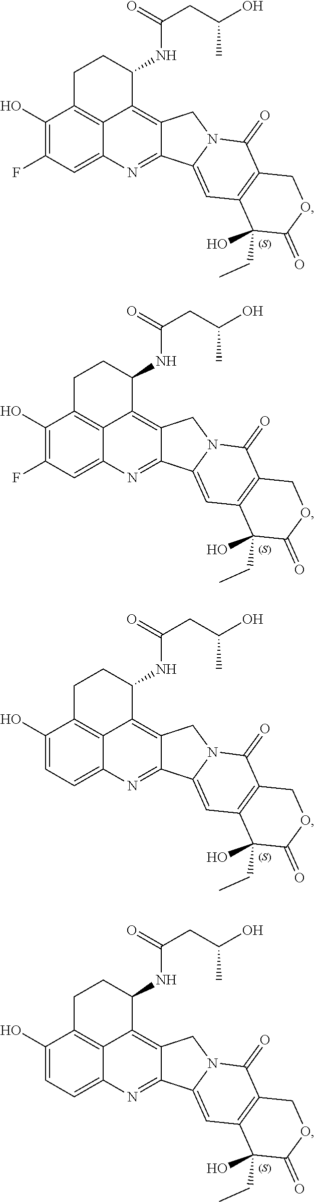

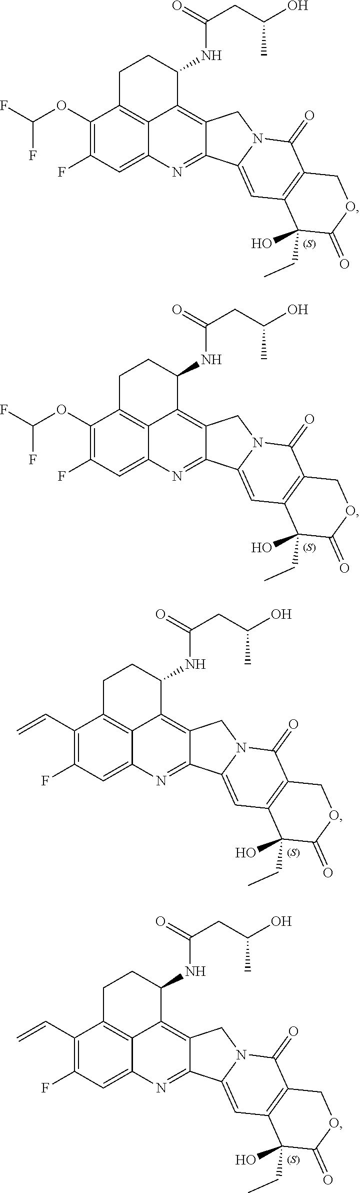

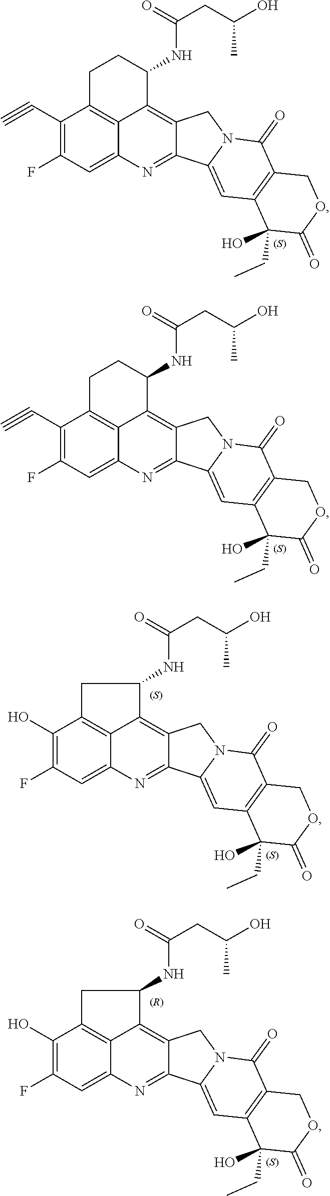

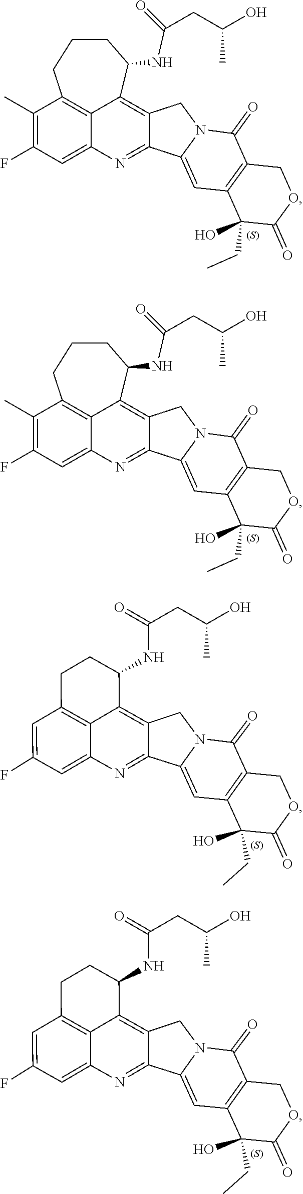

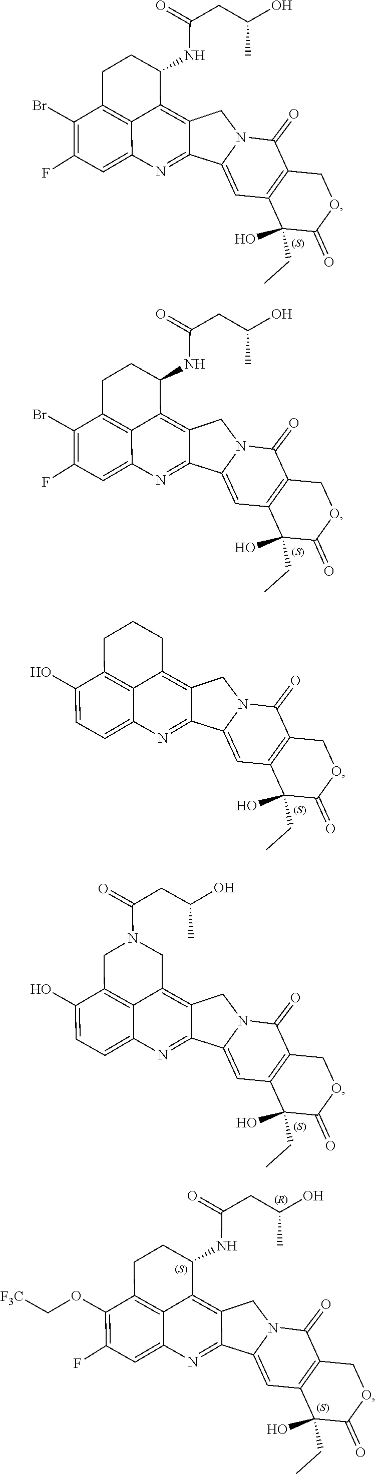

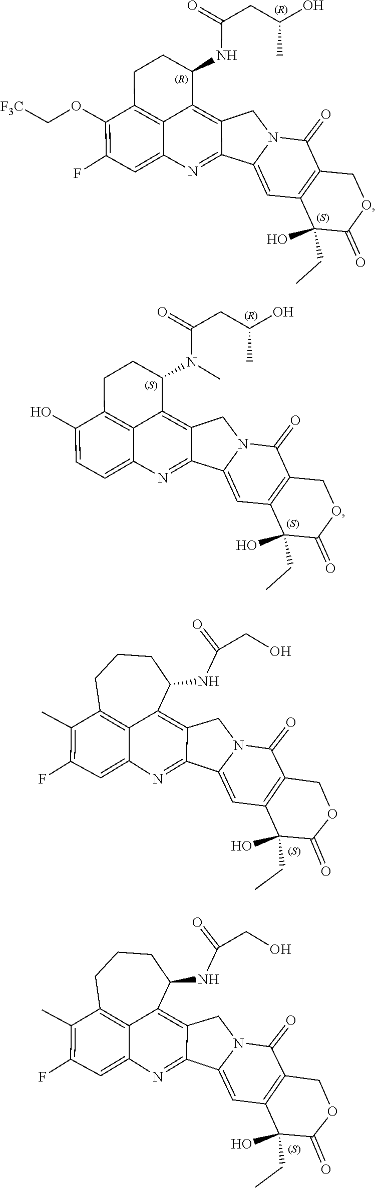

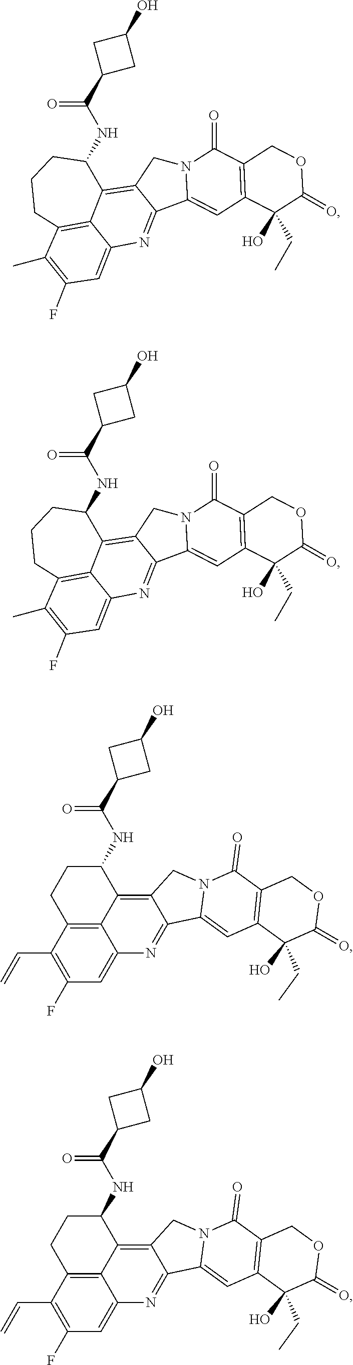 [SMILES: CC[C@@]1(O)C(=O)OCc2c1cc1n(c2=O)Cc2c-1nc1cc(F)c(C)c3c1c2[C@@H](NC(=O)[C@H]1C[C@@H](O)C1)CCC3 ; CC[C@@]1(O)C(=O)OCc2c1cc1n(c2=O)Cc2c-1nc1cc(F)c(C)c3c1c2[C@H](NC(=O)[C@H]1C[C@@H](O)C1)CCC3 ; C=Cc1c(F)cc2nc3c(c4c2c1CC[C@@H]4NC(=O)[C@H]1C[C@@H](O)C1)Cn1c-3cc2c(c1=O)COC(=O)[C@]2(O)CC ; C=Cc1c(F)cc2nc3c(c4c2c1CC[C@H]4NC(=O)[C@H]1C[C@@H](O)C1)Cn1c-3cc2c(c1=O)COC(=O)[C@]2(O)CC]

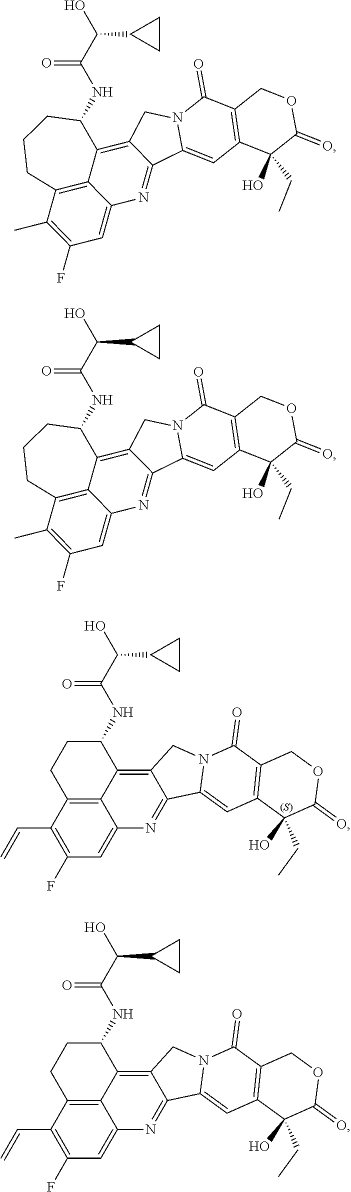

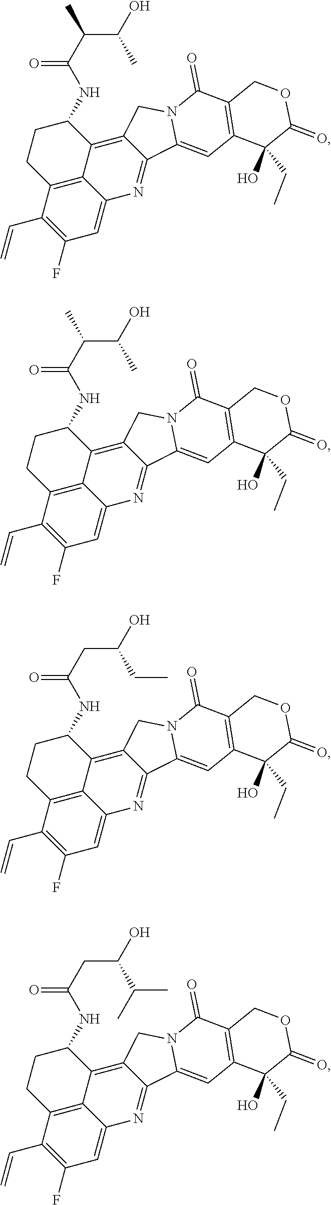

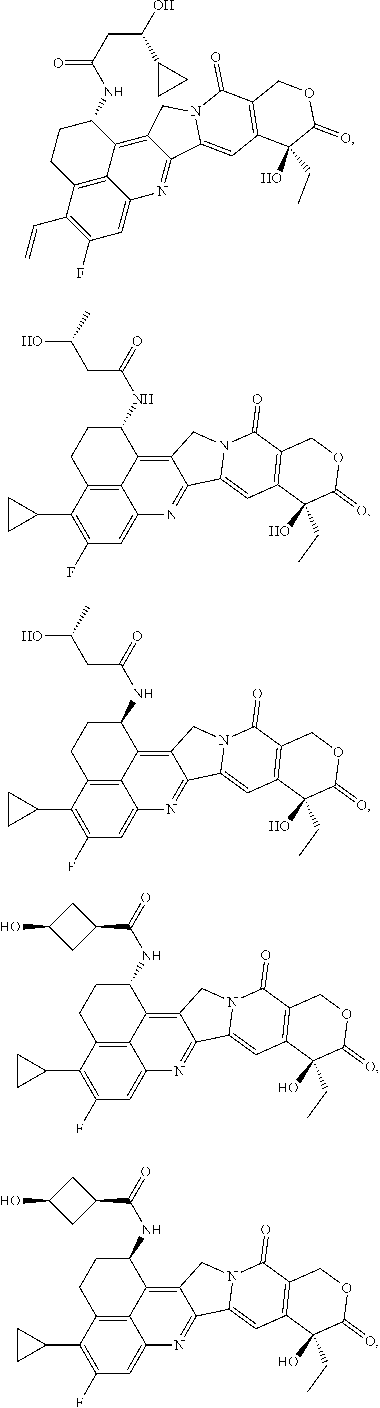

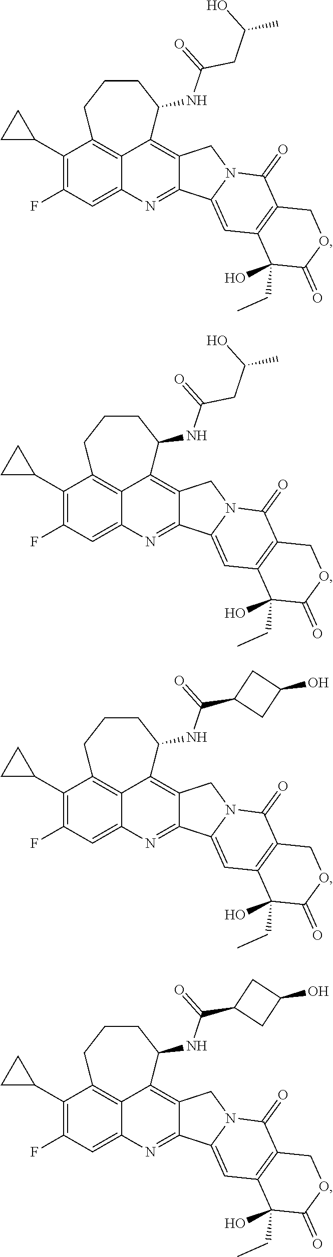

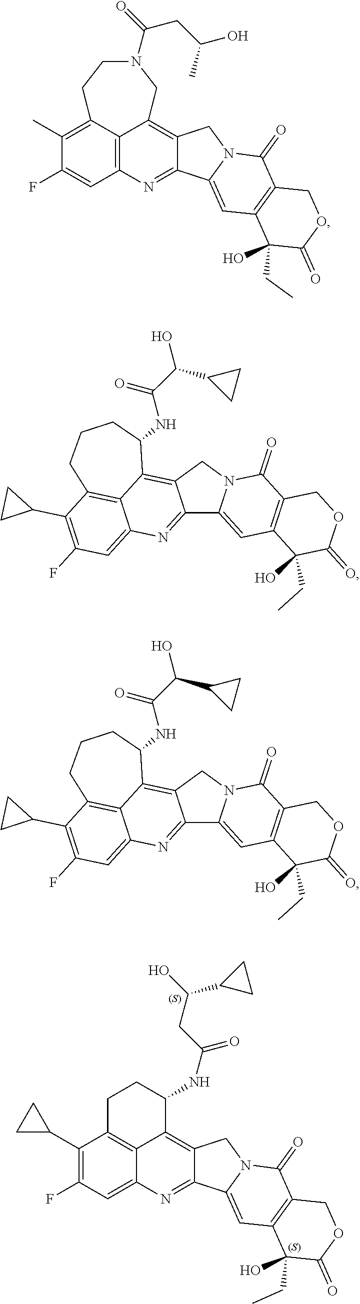

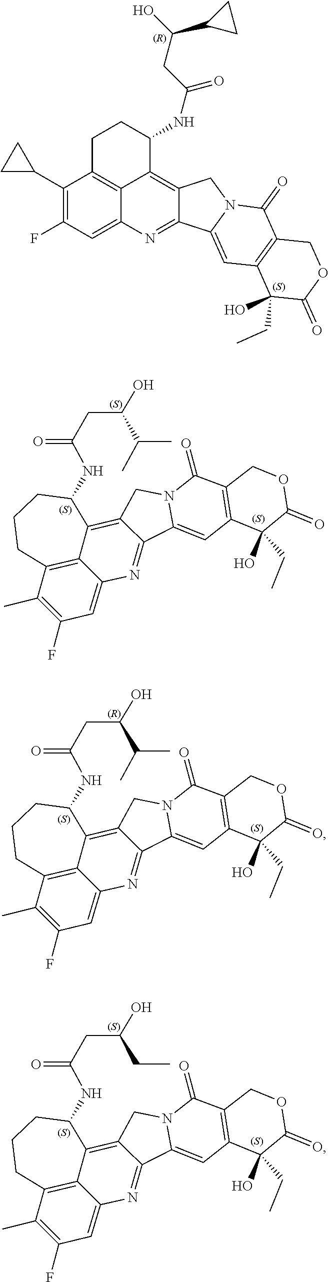

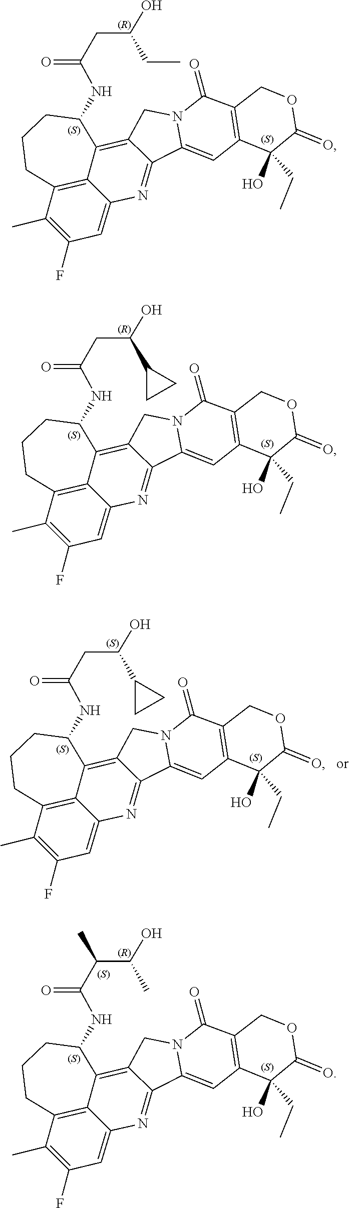

8. A structural fragment D, having the structure of the compound represented by formula I according to claim 1, a racemate, a stereoisomer, a tautomer, an isotopically labeled compound, a solvate, a polymorph, a pharmaceutically acceptable salt, or a prodrug compound thereof after dehydrogenation,
preferably, the D has a structure as shown below:

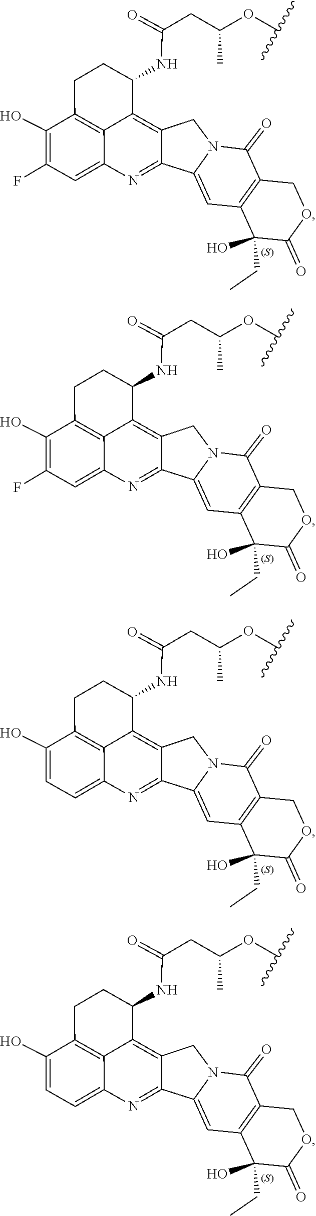

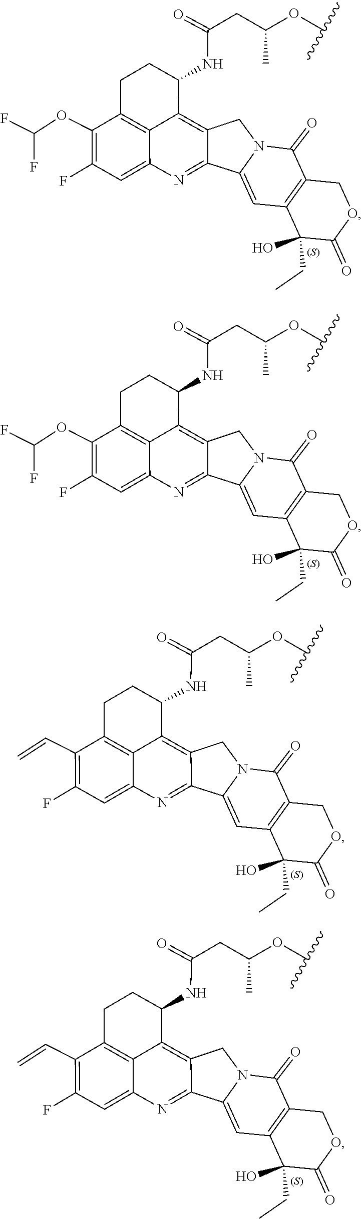

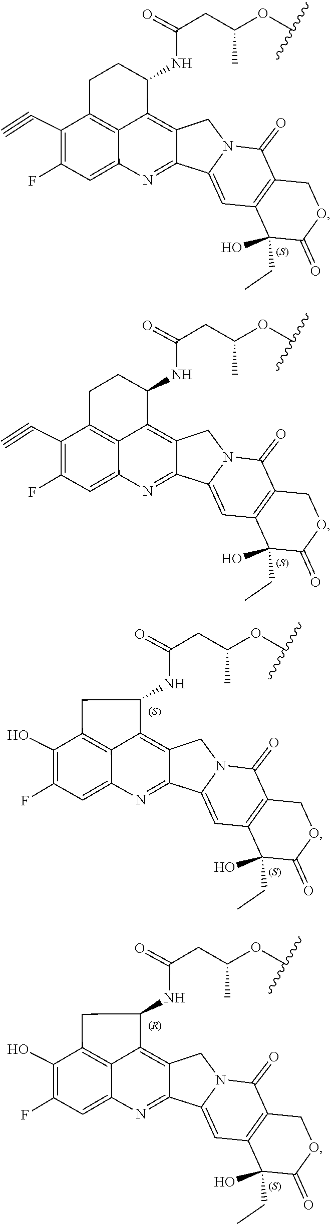

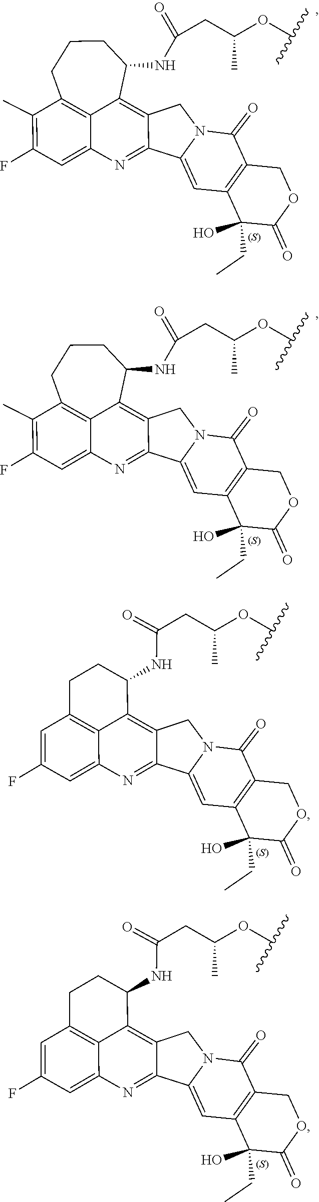

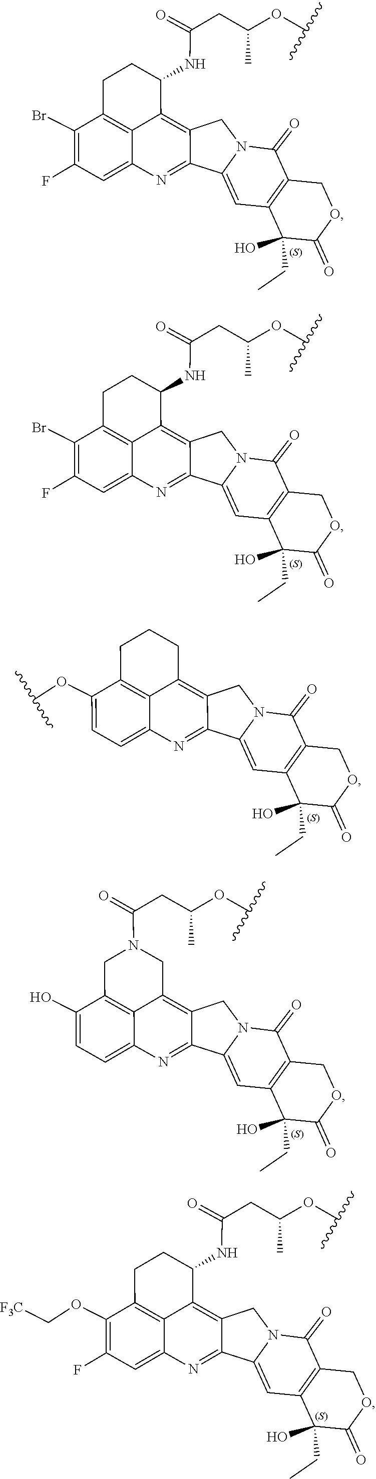

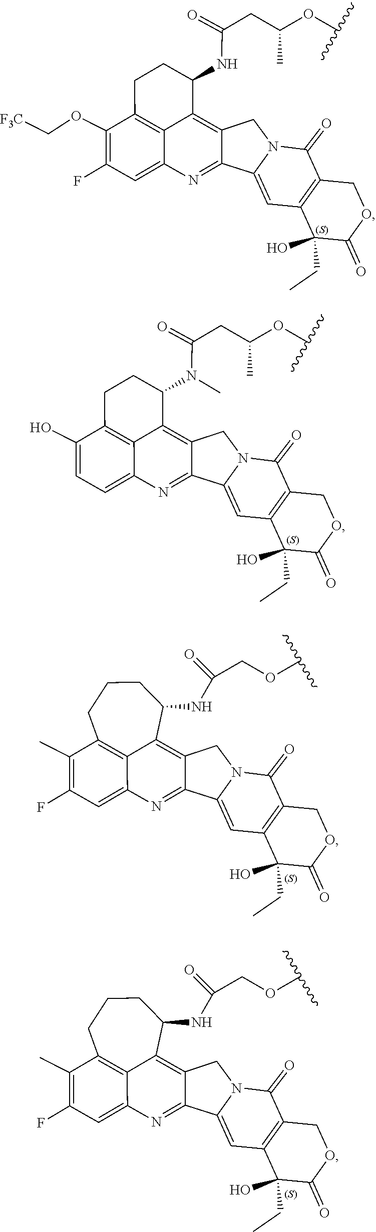

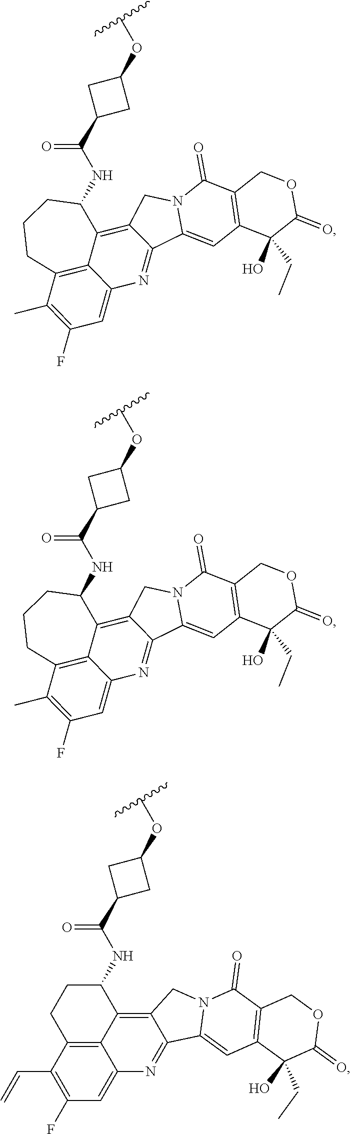 [SMILES: *O[C@H]1C[C@@H](C(=O)N[C@H]2CCCc3c(C)c(F)cc4nc5c(c2c34)Cn2c-5cc3c(c2=O)COC(=O)[C@]3(O)CC)C1 ; *O[C@H]1C[C@@H](C(=O)N[C@@H]2CCCc3c(C)c(F)cc4nc5c(c2c34)Cn2c-5cc3c(c2=O)COC(=O)[C@]3(O)CC)C1 ; *O[C@H]1C[C@@H](C(=O)N[C@H]2CCc3c(C=C)c(F)cc4nc5c(c2c34)Cn2c-5cc3c(c2=O)COC(=O)[C@]3(O)CC)C1]

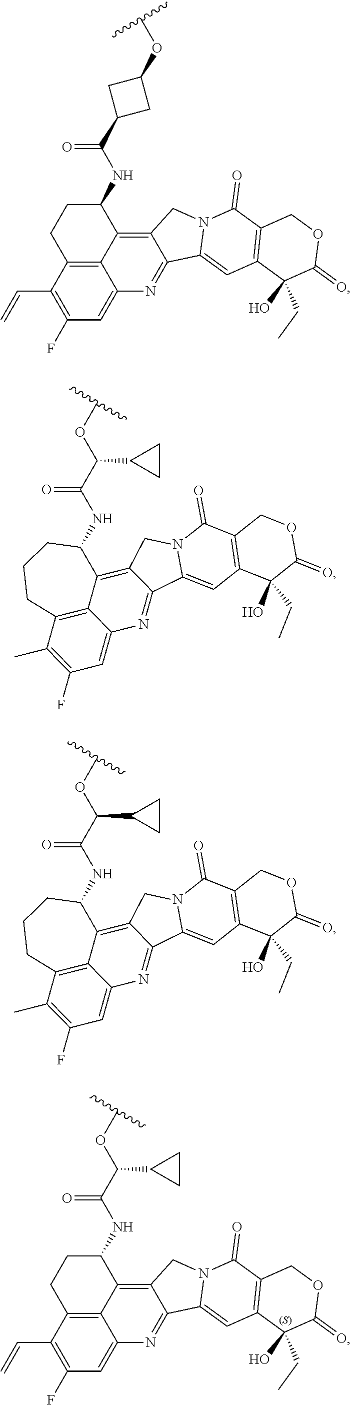

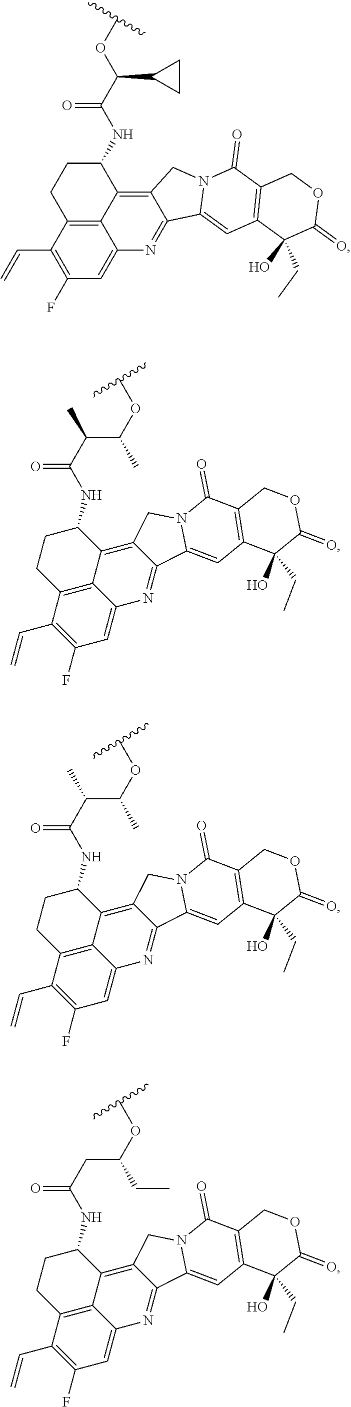 [SMILES: *O[C@H](C(=O)N[C@H]1CCc2c(C=C)c(F)cc3nc4c(c1c23)Cn1c-4cc2c(c1=O)COC(=O)[C@]2(O)CC)C1CC1 ; *O[C@H](C)[C@H](C)C(=O)N[C@H]1CCc2c(C=C)c(F)cc3nc4c(c1c23)Cn1c-4cc2c(c1=O)COC(=O)[C@]2(O)CC ; *O[C@H](C)[C@@H](C)C(=O)N[C@H]1CCc2c(C=C)c(F)cc3nc4c(c1c23)Cn1c-4cc2c(c1=O)COC(=O)[C@]2(O)CC ; *O[C@H](CC)CC(=O)N[C@H]1CCc2c(C=C)c(F)cc3nc4c(c1c23)Cn1c-4cc2c(c1=O)COC(=O)[C@]2(O)CC]

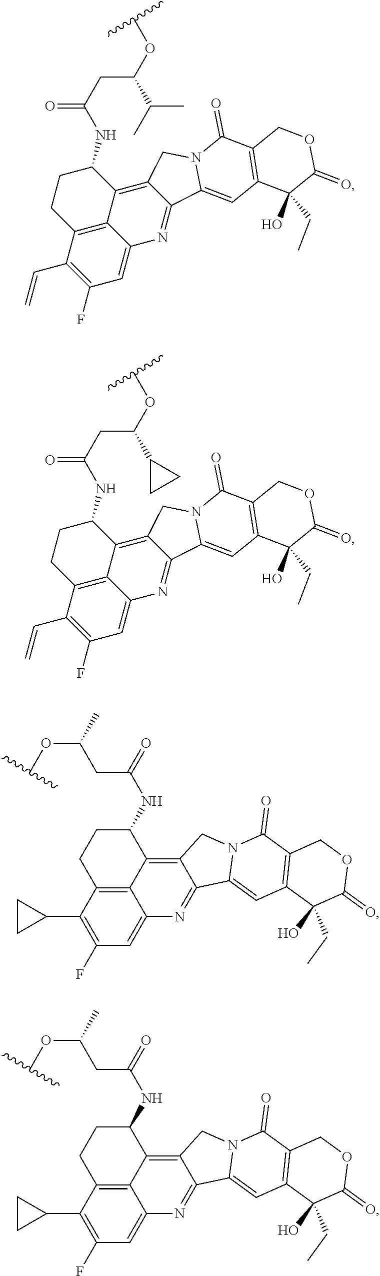

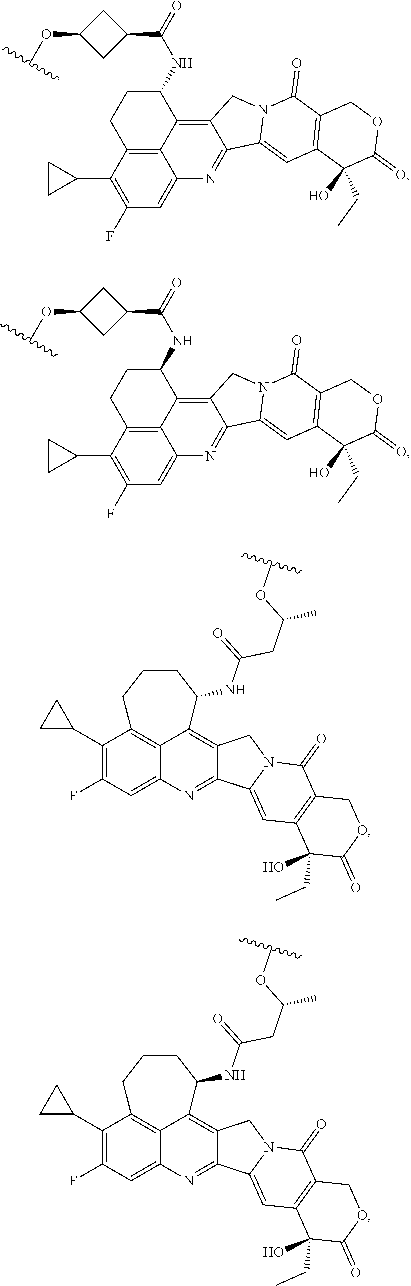 [SMILES: *O[C@H]1C[C@@H](C(=O)N[C@H]2CCc3c(C4CC4)c(F)cc4nc5c(c2c34)Cn2c-5cc3c(c2=O)COC(=O)[C@]3(O)CC)C1 ; *O[C@H]1C[C@@H](C(=O)N[C@@H]2CCc3c(C4CC4)c(F)cc4nc5c(c2c34)Cn2c-5cc3c(c2=O)COC(=O)[C@]3(O)CC)C1 ; *O[C@H](C)CC(=O)N[C@H]1CCCc2c(C3CC3)c(F)cc3nc4c(c1c23)Cn1c-4cc2c(c1=O)COC(=O)[C@]2(O)CC ; *O[C@H](C)CC(=O)N[C@@H]1CCCc2c(C3CC3)c(F)cc3nc4c(c1c23)Cn1c-4cc2c(c1=O)COC(=O)[C@]2(O)CC]

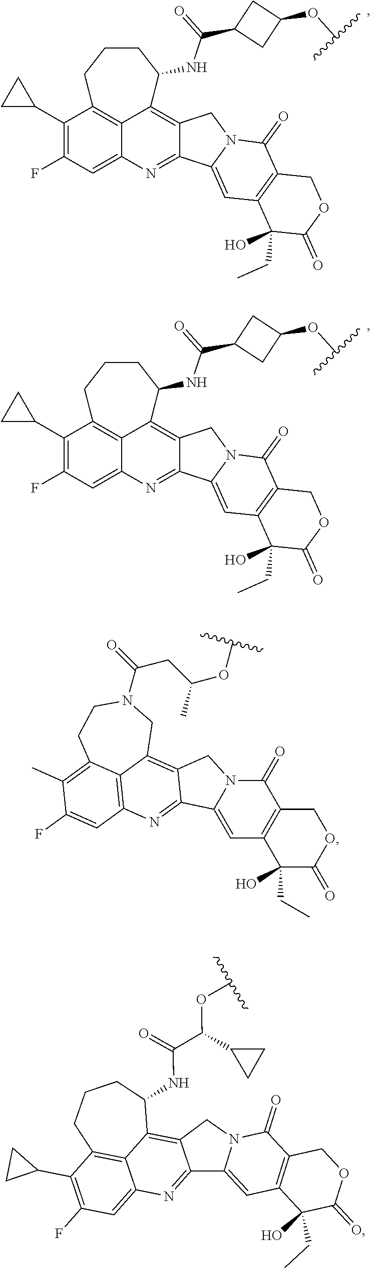

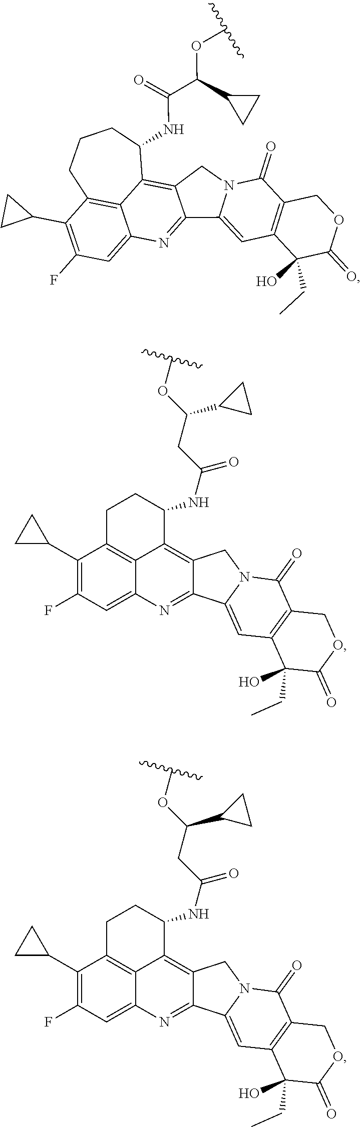 [SMILES: *O[C@H](C(=O)N[C@H]1CCCc2c(C3CC3)c(F)cc3nc4c(c1c23)Cn1c-4cc2c(c1=O)COC(=O)[C@]2(O)CC)C1CC1 ; *O[C@@H](CC(=O)N[C@H]1CCc2c(C3CC3)c(F)cc3nc4c(c1c23)Cn1c-4cc2c(c1=O)COC(=O)[C@]2(O)CC)C1CC1 ; *O[C@H](CC(=O)N[C@H]1CCc2c(C3CC3)c(F)cc3nc4c(c1c23)Cn1c-4cc2c(c1=O)COC(=O)[C@]2(O)CC)C1CC1]

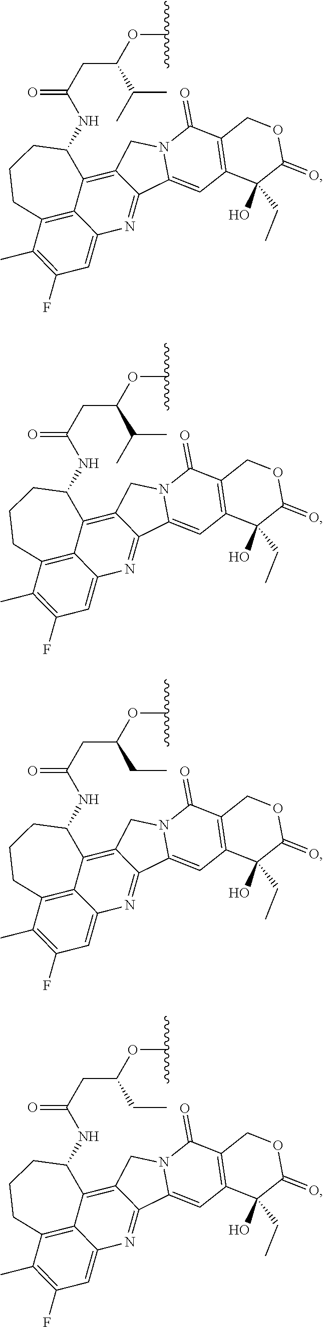 [SMILES: *O[C@@H](CC(=O)N[C@H]1CCCc2c(C)c(F)cc3nc4c(c1c23)Cn1c-4cc2c(c1=O)COC(=O)[C@]2(O)CC)C(C)C ; *O[C@H](CC(=O)N[C@H]1CCCc2c(C)c(F)cc3nc4c(c1c23)Cn1c-4cc2c(c1=O)COC(=O)[C@]2(O)CC)C(C)C ; *O[C@@H](CC)CC(=O)N[C@H]1CCCc2c(C)c(F)cc3nc4c(c1c23)Cn1c-4cc2c(c1=O)COC(=O)[C@]2(O)CC ; *O[C@H](CC)CC(=O)N[C@H]1CCCc2c(C)c(F)cc3nc4c(c1c23)Cn1c-4cc2c(c1=O)COC(=O)[C@]2(O)CC]

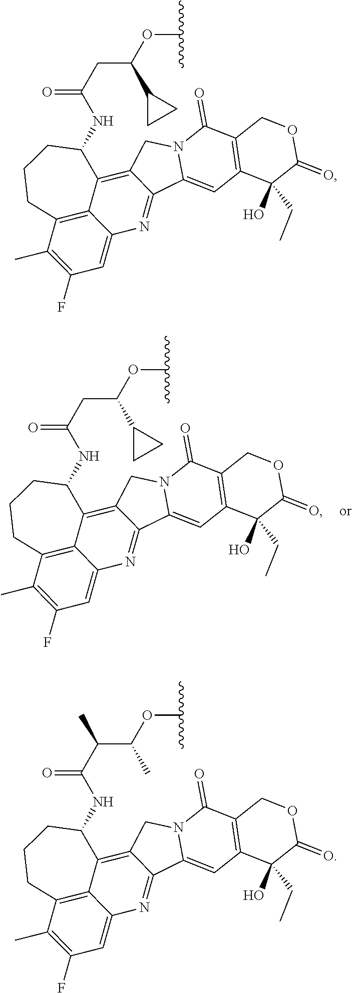 [SMILES: *O[C@H](CC(=O)N[C@H]1CCCc2c(C)c(F)cc3nc4c(c1c23)Cn1c-4cc2c(c1=O)COC(=O)[C@]2(O)CC)C1CC1 ; *O[C@@H](CC(=O)N[C@H]1CCCc2c(C)c(F)cc3nc4c(c1c23)Cn1c-4cc2c(c1=O)COC(=O)[C@]2(O)CC)C1CC1 ; *O[C@H](C)[C@H](C)C(=O)N[C@H]1CCCc2c(C)c(F)cc3nc4c(c1c23)Cn1c-4cc2c(c1=O)COC(=O)[C@]2(O)CC]

9. A compound represented by formula V, or a racemate, a stereoisomer, a tautomer, an isotopically labeled compound, a solvate, a polymorph, a pharmaceutically acceptable salt, or a prodrug compound thereof: 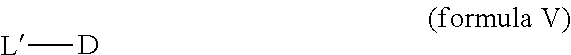
wherein L′ is a linker containing a linker site M capable of reacting with an antibody or an antigen-binding fragment thereof, and after L′-D reacts with the antibody or the antigen-binding fragment thereof, the L′ thereof forms a linker L;
preferably, the L′ comprises a peptide residue L₁ and a fragment L₂ in which the peptide residue is connected to D; and
the D is as defined in claim 8.

10. An antibody-drug conjugate represented by formula VI, 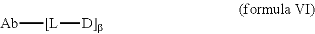
wherein Ab is an antibody or an antigen-binding fragment thereof, D is as defined in claim 8, L is a linker linking the Ab to the D, and β is an integer or a decimal selected from 1-10.

11. A method for preparing the compound according to claim 1, comprising solution I or solution II below:
solution I: comprising steps of:
(1) removing a protecting group PG₄ from compound I-41 to give compound I-42; and
(2) allowing the compound I-42 to react with compound I-43 to give the compound represented by formula I; 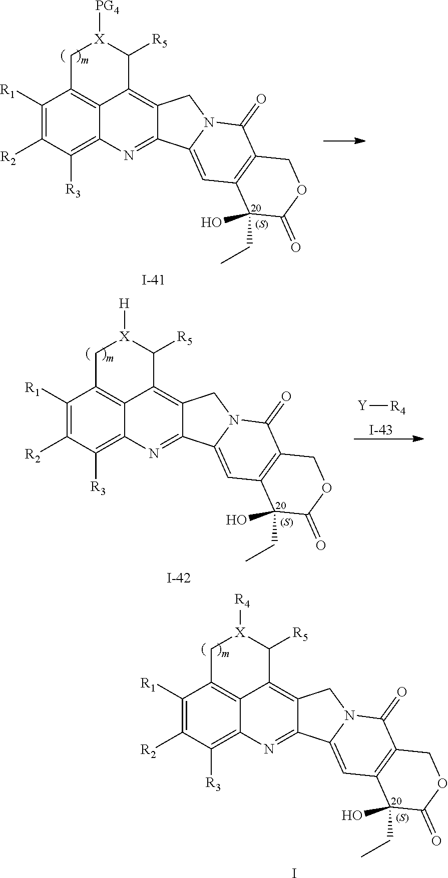
wherein the R₁, R₂, R₃, R₄, R₅, X, and m are as defined in claim 1; Y is selected from a leaving group, for example, OH, Cl, Br, or I; and the PG₄ is selected from an amino protecting group, for example, Fmoc, Boc, Bn, or Cbz; or
solution II: comprising steps of:
(1) removing a protecting group PG₅ from compound I-51 to give compound I-52; and
(2) allowing the compound I-52 to react with compound I-53 to give the compound represented by formula I; 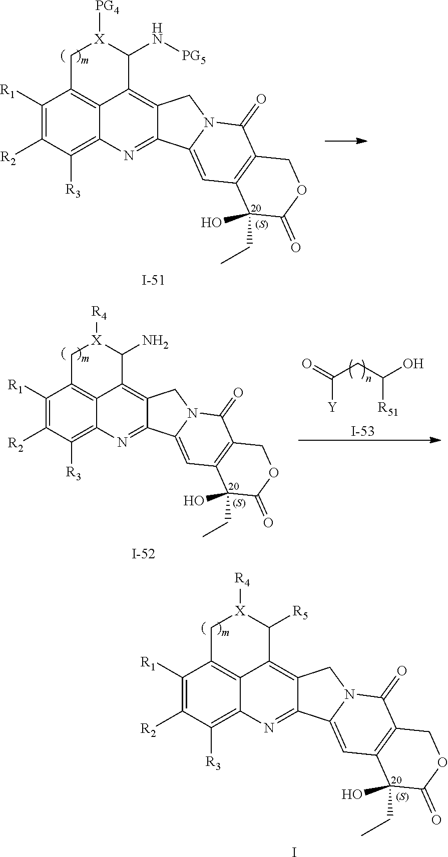
wherein the R₁, R₂, R₃, R₄, R₅, R₅₁, X, m, and n are as defined in claim 1; Y is selected from a leaving group, for example, OH, Cl, Br, or I; and the PG₅ is selected from an amino protecting group, for example, Fmoc, Boc, Bn, or Cbz.

12. A pharmaceutical composition, comprising a therapeutically effective amount of the compound according to claim 1, a racemate, a stereoisomer, a tautomer, an isotopically labeled compound, a solvate, a polymorph, a pharmaceutically acceptable salt, or a prodrug compound thereof.

13. (canceled)

14. A method for treating a tumor disease, comprising administering to a patient a prophylactically or therapeutically effective amount of the compound represented by formula I according to claim 1, a racemate, a stereoisomer, a tautomer, an isotopically labeled compound, a solvate, a polymorph, a pharmaceutically acceptable salt, or a prodrug compound thereof; wherein
preferably, the tumor comprises breast cancer, gastric cancer, lung cancer, colorectal cancer, large intestine cancer, ovarian cancer, liver cancer, kidney cancer, esophageal cancer, cervical cancer, bladder cancer, pancreatic cancer, prostate cancer, nasopharyngeal carcinoma, melanoma, or leukemia.

15. A method for treating a tumor disease, comprising administering to a patient a prophylactically or therapeutically effective amount of the compound represented by formula V according to claim 9, a racemate, a stereoisomer, a tautomer, an isotopically labeled compound, a solvate, a polymorph, a pharmaceutically acceptable salt, or a prodrug compound thereof, wherein
preferably, the tumor comprises breast cancer, gastric cancer, lung cancer, colorectal cancer, large intestine cancer, ovarian cancer, liver cancer, kidney cancer, esophageal cancer, cervical cancer, bladder cancer, pancreatic cancer, prostate cancer, nasopharyngeal carcinoma, melanoma, or leukemia.

16. A method for treating a tumor disease, comprising administering to a patient a prophylactically or therapeutically effective amount of the antibody-drug conjugate represented by formula VI according to claim 10; wherein
preferably, the tumor comprises breast cancer, gastric cancer, lung cancer, colorectal cancer, large intestine cancer, ovarian cancer, liver cancer, kidney cancer, esophageal cancer, cervical cancer, bladder cancer, pancreatic cancer, prostate cancer, nasopharyngeal carcinoma, melanoma, or leukemia.

17. A pharmaceutical composition, comprising a therapeutically effective amount of the antibody-drug conjugate according to claim 10.
